(19) United States
(12) Patent Application Publication
(10) Pub. No.: US 2019/0241573 A1
(43) Pub. Date: Aug. 8, 2019

(54) ISOQUINOLINE DERIVATIVES AS PERK INHIBITORS

(71) Applicant: GlaxoSmithKline Intellectual Property Development Limited, Brentford, Middlesex (GB)

(72) Inventors: Jeffrey Michael AXTEN, Collegeville, PA (US); Raghava Reddy KETHIRI, Bangalore (IN); Rajendra KRISTAM, Bangalore (IN); Chandregowda VENKATESHAPPA, Bangalore (IN)

(73) Assignee: GlaxoSmithKline Intellectual Property Development Limited, Brentford, Middlesex (GB)

(21) Appl. No.: 16/318,408

(22) PCT Filed: Jul. 18, 2017
(86) PCT No.: PCT/IB2017/054332

§ 371 Date: Jan. 17, 2019

(30) Foreign Application Priority Data
Jul. 20, 2016 (IN) 201611024887

(51) Int. Cl. C07D 487/04; C07D 471/04; A61P 35/00; A61P 25/28; A61P 27/06

(52) U.S. Cl. CPC C07D 487/04; C07D 471/04; A61K 45/06; A61P 25/28; A61P 27/06; A61P 35/00

(57) ABSTRACT

The invention is directed to substituted isoquinoline derivatives and uses thereof. Specifically, the invention is directed to compounds according to Formula I and the use of compounds of Formula (I) in treating disease states:

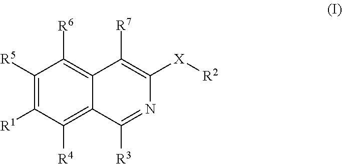

wherein R¹, R², R³, R⁴, R⁵, R⁶, R⁷ and X are as defined herein.

The compounds of the invention are inhibitors of PERK and can be useful in the treatment of cancer, pre-cancerous syndromes and diseases associated with activated unfolded protein response pathways, such as Alzheimer's disease, spinal cord injury, traumatic brain injury, ischemic stroke, stroke, Parkinson disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, ather osclerosis, ocular diseases, arrhythmias, in organ transplantation and in the transportation of organs for transplantation. Accordingly, the invention is further directed to pharmaceutical compositions comprising a compound of the invention. The invention is still further directed to methods of inhibiting PERK activity and treatment of disorders associated therewith using a compound of the invention or a pharmaceutical composition comprising a compound of the invention.

### FIELD OF THE INVENTION

The present invention relates to substituted isoquinoline derivatives that are inhibitors of the activity of the protein kinase R (PKR)-like ER kinase, PERK. The present invention also relates to pharmaceutical compositions comprising such compounds and methods of using such compounds in the treatment of cancer, pre-cancerous syndromes and diseases/injuries associated with activated unfolded protein response pathways, such as Alzheimer's disease, spinal cord injury, traumatic brain injury, ischemic stroke, stroke, Parkinson's disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation.

### BACKGROUND OF THE INVENTION

The unfolded protein response (UPR) is a signal transduction pathway that allows cells to survive stress caused by the presence of misfolded or unfolded proteins or protein aggregates (Walter and Ron, 2011), (Hetz, 2012). Environmental stresses that perturb protein folding and maturation in the endoplasmic reticulum (ER) also can lead to activation of the UPR (Feldman et al., 2005), (Koumenis and Wouters, 2006). UPR activating stress stimuli include hypoxia, disruption of protein glycosylation (glucose deprivation), depletion of luminal ER calcium, or changes in ER redox status, among others (Ma and Hendershot, 2004), (Feldman et al., 2005). These perturbations result in disruption of ER redox homeostasis and the accumulation of unfolded or mis-folded proteins in the ER. Cellular responses include transcriptional reprogramming to increase the level of chaperone proteins to enhance protein re-folding, degradation of the mis-folded proteins, and translational arrest to decrease the burden of client proteins entering the ER (Ron, D. 2002), (Harding et al., 2002). These pathways also regulate cell survival by modulating apoptosis (Ma and Hendershot, 2004), (Feldman et al., 2005), and autophagy (Rouschop et al. 2010), and can trigger cell death under conditions of prolonged ER stress (Woehlbier and Hetz, 2011).

Three ER membrane proteins have been identified as primary effectors of the UPR: protein kinase R (PKR)-like ER kinase [PERK, also known as eukaryotic initiation factor 2A kinase 3 (EIF2AK3), pancreatic ER kinase, or pancreatic elF2α kinase (PEK)], inositol-requiring gene 1 α/β (IRE1), and activating transcription factor 6 (ATF6) (Ma and Hendershot, 2004), (Hetz, 2012). Under normal conditions these proteins are held in the inactive state through binding of the ER chaperone GRP78 (BiP) to their luminal sensor domain. Accumulation of unfolded proteins in the ER leads to release of GRP78 from these sensors resulting in activation of these UPR effectors (Ma et al., 2002), (Hetz, 2012).

PERK is a type I ER membrane protein containing a stress-sensing domain facing the ER lumen, a transmembrane segment, and a cytosolic kinase domain (Shi et al., 1998), (Harding et al., 1999), (Sood et al., 2000). Release of GRP78 from the stress-sensing domain of PERK results in oligomerization and autophosphorylation at multiple serine, threonine and tyrosine residues (Ma et al., 2001), (Su et al., 2008). Phenotypes of PERK knockout mice include diabetes, due to loss of pancreatic islet cells, skeletal abnormalities, and growth retardation (Harding et al., 2001), (Zhang et al., 2006), (Iida et al., 2007). These features are similar to those seen in patients with Wolcott-Rallison syndrome, who carry germline mutations in the PERK gene (Julier and Nicolino, 2010).

The major substrate for PERK is the eukaryotic initiation factor 2α (elF2α), which PERK phosphorylates at serine-51 (Marciniak et al., 2006) in response to ER stress or treatment with pharmacological inducers of ER stress such as thapsigargin and tunicamycin. This site is also phosphorylated by other EIF2AK family members [(general control non-derepressed 2 (GCN2), PKR, and heme-regulated kinase (HRI)] in response to different stimuli.

Phosphorylation of elF2α converts it to an inhibitor of the guanine nucleotide exchange factor (GEF) elF2B which is required for efficient turnover of GDP for GTP in the elF2 protein synthesis complex. As a result, the inhibition of elF2B by P-elF2α causes a decrease in translation initiation and global protein synthesis (Harding et al. 2002).

Paradoxically, translation of specific mRNAs is enhanced when the UPR is activated and elF2α is phosphorylated. For example, the transcription factor ATF4 has 5′-upstream open reading frames (uORFs) that normally represses ATF4 synthesis during normal global protein synthesis. However, when PERK is activated under stress and P-elF2α inhibits elF2B, low levels of ternary complex allows for selective enhanced translation of ATF4 (Jackson et al. 2010). Therefore, when ER stress ensues, PERK activation causes an increase in ATF4 translation, which transcriptionally upregulates downstream target genes such as CHOP (transcription factor C/EBP homologous protein). This transcriptional reprogramming modulates cell survival pathways and can lead to the induction of proapoptotic genes.

The activation of PERK and the UPR is associated with human neurodegenerative conditions such as Alzheimer's disease, Parkinson's disease, Huntington's disease, amyotrophic lateral sclerosis (ALS), progressive supranuclear palsy (PSP), dementias, and prion diseases including Creutzfeldt-Jakob Disease (CJD), (Doyle et al. 2011), (Paschen 2004), (Salminen et al. 2009), (Stutzbach et al. 2013). The common hallmark of all these diseases is the presence of malformed/misfolded or aggregated protein deposits (e.g tau tangles, Lewy bodies, α-synuclein, Aβ plaques, mutant prion proteins) believed to contribute to the underlying disease pathophysiology, neuron loss, and cognitive decline (Prusiner, 2012), (Doyle et al. 2011). The fate of a cell (e.g a neuron) enduring unfolded or malfolded protein stress is under control of PERK. A cell enduring ER stress may restore proteostasis and return to normal, or if the stress is insurmountable, sustained PERK activation may lead to cell death through ATF4/CHOP signaling coupled with the inability to synthesize vital proteins because of the persistent translational repression. Activated PERK and associated biological markers of PERK activation are detected in post-mortem brain tissue of Alzheimer's disease patients and in human prion disease (Ho et al. 2012), (Hoozemans et al, 2009) (Unterberger et al. 2006). Furthermore, P-elF2α (the product of PERK activation) correlates with levels of BACE1 in post-mortem brain tissue of Alzheimer's disease patients (O'Connor et al. 2008). Recently, the small molecule PERK inhibitor GSK2606414 was shown to provide a neuroprotective effect and prevent clinical signs of disease in prion infected mice (Moreno et al. 2013), consistent with previous results derived from genetic manipulation of the UPR/PERK/elF2α pathway (Moreno et al. 2012). Involvement of the pathway in ALS (Kanekura et. al., 2009 and Nassif et. al. 2010), spinal cord injury (Ohri et al. 2011) and traumatic brain injury (Tajiri et al. 2004) is also reported. Taken together these data suggest that the UPR and PERK represent a promising node of drug intervention as a means to halt or reverse the clinical progression and associated cognitive impairments of a wide range of neurodegenerative diseases.

Tumor cells experience episodes of hypoxia and nutrient deprivation during their growth due to inadequate blood supply and aberrant blood vessel function (Brown and Wilson, 2004), (Blais and Bell, 2006). Thus, they are likely to be dependent on active UPR signaling to facilitate their growth. Consistent with this, mouse fibroblasts derived from PERK−/−, XBP1−/−, and ATF4−/− mice, and fibroblasts expressing mutant elF2α show reduced clonogenic growth and increased apoptosis under hypoxic conditions in vitro and grow at substantially reduced rates when implanted as tumors in nude mice (Koumenis et al., 2002), (Romero-Ramirez et al., 2004), (Bi et al., 2005). Human tumor cell lines carrying a dominant negative PERK that lacks kinase activity also showed increased apoptosis in vitro under hypoxia and impaired tumor growth in vivo (Bi et al., 2005). In these studies, activation of the UPR was observed in regions within the tumor that coincided with hypoxic areas. These areas exhibited higher rates of apoptosis compared to tumors with intact UPR signaling. Further evidence supporting the role of PERK in promoting tumor growth is the observation that the number, size, and vascularity of insulinomas arising in transgenic mice expressing the SV40-T antigen in the insulin-secreting beta cells, was profoundly reduced in PERK^{-/-} mice compared to wild-type control (Gupta et al., 2009). Activation of the UPR has also been observed in clinical specimens. Human tumors, including those derived from cervical carcinomas, glioblastomas (Bi et al., 2005), lung cancers (Jorgensen et al., 2008) and breast cancers (Ameri et al., 2004), (Davies et al., 2008) show elevated levels of proteins involved in UPR, compared to normal tissues. Therefore, inhibiting the unfolded protein response with compounds that block the activity of PERK and other components of the UPR is expected to have utility as anticancer agents. Recently, this hypothesis was supported by two small molecule inhibitors of PERK that were shown to inhibit the growth of human tumor xenografts in mice (Axten et al. 2012 and Atkins et al. 2013).

Loss of endoplasmic reticulum homeostasis and accumulation of misfolded proteins can contribute to a number of disease states (Wek and Cavener 2007), (Zhang and Kaufman 2006). Inhibitors of PERK may be therapeutically useful for the treatment of a variety of human diseases such as Alzheimer's disease and frontotemporal dementias, Parkinson's disease, Huntington's disease, amyotrophic lateral sclerosis (ALS), progressive supranuclear palsy (PSP), and other tauopathies such chronic traumatic encephalopathy (CTE) (Nijholt, D. A., et al. 2012), (Lucke-Wold, B. P., et al. 2016), spinal cord injury, traumatic brain injury, stroke, Creutzfeldt-Jakob Disease (CJD) and related prion diseases, such as fatal familial insomnia (FFI), Gerstmann-Straussler-Scheinker Syndrome, and vanishing white matter (VWM) disease. Inhibitors of PERK may also be useful for effective treatment of cancers, particularly those derived from secretory cell types, such as pancreatic and neuroendocrine cancers, multiple myeloma, or for use in combination as a chemosensitizerto enhance tumor cell killing. A PERK inhibitor may also be useful for myocardial infarction, cardiovascular disease, atherosclerosis (McAlpine et al., 2010, Civelek et al. 2009, Liu and Dudley 2016), arrhythmias, and kidney disease (Dickhout et al., 2011, Cybulsky, A. V., et al. 2005). A PERK inhibitor may also be useful in stem cell or organ transplantation to prevent damage to the organ and in the transportation of organs for transplantation (Inagi et al., 2014), (Cunard, 2015), (Dickhout et al., 2011), (van Galen, P., et al. (2014). A PERK inhibitor is expected to have diverse utility in the treatment of numerous diseases in which the underlying pathology and symptoms are associated with dysregulaton of the unfolded protein response.

### REFERENCES

Ameri, K., Lewis, C. E., Raida, M., Sowter, H., Hai, T., and Harris, A. L. (2004). Anoxic induction of ATF-4 through HIF-1-independent pathways of protein stabilization in human cancer cells, Blood 103, 1876-82.
Atkins, C.; Liu, Q.; Minthorn, E.; Zhang, S.; Figueroa, D. J.; Moss, K.; Stanley, T. B.; Sanders, B.; Goetz, A.; Gaul, N.; Choudhry, A. E.; Alsaid, H.; Jucker, B. M.; Axten, J. M.; Kumar, R. (2013) Characterization of a novel PERK kinase inhibitor with anti-tumor and anti-angiogenic activity. Cancer Res. 74, 1993-2002.
Axten, J. M.; Medina, J. R.; Feng, Y.; Shu, A.; Romeril, S. P.; Grant, S. W.; Li, W. H. H.; Heerding, D. A.; Minthorn, E.; Mencken, T.; Atkins, C.; Liu, Q.; Rabindran, S.; Kumar, R.; Hong, X.; Goetz, A.; Stanley, T.; Taylor, J. D.; Sigethy, S. D.; Tomberlin, G. H.; Hassell, A. M.; Kahler, K. M.; Shewchuk, L. M.; Gampe, R. T. (2012) Discovery of 7-methyl-5-(1-{[3-(trifluoromethyl)phenyl]acetyl}-2,3-dihydro-1H-indol-5-yl)-7H-pyrrolo[2,3-d]pyrimidin-4-amine (GSK2606414), a potent and selective first-in-class inhibitor of protein kinase R (PKR)-like endoplasmic reticulum kinase (PERK). J. Med. Chem. 55, 7193-7207.
Bi, M., Naczki, C., Koritzinsky, M., Fels, D., Blais, J., Hu, N., Harding, H., Novoa, I., Varia, M., Raleigh, J., et al. (2005). ER stress-regulated translation increases tolerance to extreme hypoxia and promotes tumor growth, EMBO J 24, 3470-81.
Blais, J., and Bell, J. C. (2006). Novel therapeutic target: the PERKs of inhibiting the integrated stress response, Cell Cycle 5, 2874-7.
Civelek, M., et al. (2009). Chronic endoplasmic reticulum stress activates unfolded protein response in arterial endothelium in regions of susceptibility to atherosclerosis. Circ Res 105(5). 453-461.
Cunard, R (2015). Endoplasmic Reticulum Stress in the Diabetic Kidney, the Good, the Bad and the Ugly, J. Clin. Med. 4, 715-740.
Cybulsky, A. V., et al. (2005). Role of the endoplasmic reticulum unfolded protein response in glomerular epithelial cell injury. J Biol Chem 280(26): 24396-24403.
Davies, M. P., Barraclough, D. L., Stewart, C., Joyce, K. A., Eccles, R. M., Barraclough, R., Rudland, P. S., and Sibson, D. R. (2008). Expression and splicing of the unfolded protein response gene XBP-1 are significantly associated with clinical outcome of endocrine-treated breast cancer, Int J Cancer 123, 85-8.
Dickhout, J. G.; Carlisle, R. E.; Austin, R. C.; (2011). Interrelationship Between Cardiac Hypertrophy, Heart Failure, and Chronic Kidney, Circ Res. 108, 629-642.
Doyle, K. M.; Kennedy, D.; Gorman, A. M.; Gupta, S.; Healy, S. J. M.; Samali, A. (2011) Unfolded proteins and endoplasmic reticulum stress in neurodegenerative disorders J. Cell. Mol. Med. 15, 2025-2039.
Feldman, D. E., Chauhan, V., and Koong, A. C. (2005). The unfolded protein response: a novel component of the hypoxic stress response in tumors, Mol Cancer Res 3, 597-605. Gupta, S., McGrath, B., and Cavener, D. R. (2009). PERK regulates the proliferation and development of insulin-secreting beta-cell tumors in the endocrine pancreas of mice, PLoS One 4, e8008.
Hamanaka, R. B., Bennett, B. S., Cullinan, S. B., and Diehl, J. A. (2005). PERK and GCN2 contribute to elF2alpha phosphorylation and cell cycle arrest after activation of the unfolded protein response pathway, Mol Biol Cell 16, 5493-501.
Harding, H. P.; Zhang, Y.; Ron, D. (1999) Protein translation and folding are coupled by an endoplasmic-reticulumresident kinase Nature 397, 271-274.
Harding, H. P., Zeng, H., Zhang, Y., Jungries, R., Chung, P., Plesken, H., Sabatini, D. D., and Ron, D. (2001). Diabetes mellitus and exocrine pancreatic dysfunction in perk−/− mice reveals a role for translational control in secretory cell survival, Mol Cell 7, 1153-63.
Harding, H. P.; Calfon, M.; Urano, F.; Novoa, I.; Ron, D. (2002) TRANSCRIPTIONAL AND TRANSLATIONAL CONTROL IN THE MAMMALIAN UNFOLDED PROTEIN RESPONSE. Annu. Rev. Cell Dev. Biol. 18, 575-599
Hetz, C. (2012). The unfolded protein response: controlling cell fate decisions under ER stress and beyond. Nat. Rev. Mol. Cell Bio. 13, 89-102.
Ho, Y.; Yang, X.; Lau, J. C; Hung, C. H.; Wuwongse, S; Zhang, Q.; Wang, J.; Baum, L.; So, K.; Chang, R. C. (2012) Endoplasmic Reticulum Stress Induces Tau Pathology and Forms a Vicious Cycle: Implication in Alzheimer's Disease Pathogenesis J. Alzheimer's Disease 28, 839-854.
Hoozemans. J. J. M.; van Haastert, E. S.; Nijholt, D. A. T.; Rozemuller, A. J. M.; Eikelenboom, P.; Scheper, W. (2009) The Unfolded Protein Response Is Activated in Pretangle Neurons in Alzheimer's Disease Hippocampus Am. J. Path. 174, 1241-1251.
Iida, K., Li, Y., McGrath, B. C., Frank, A., and Cavener, D. R. (2007). PERK elF2 alpha kinase is required to regulate the viability of the exocrine pancreas in mice, BMC Cell Biol 8, 38.
Inagi, R., Ishimoto, Y, and Nangaku, M. (2014). Proteostasis in endoplasmic reticulum—new mechanisms in kidney disease, Nat. Rev. Nephrol. 10, 369-378.
Jackson, R. J.; Hellen, C. U. T.; Pestova, T. V. (2010) The mechanism of eukaryotic translation initiation and principles of its regulation Nat. Rev. Mol. Cell Biol. 10, 113-127.
Jorgensen, E., Stinson, A., Shan, L., Yang, J., Gietl, D., and Albino, A. P. (2008). Cigarette smoke induces endoplasmic reticulum stress and the unfolded protein response in normal and malignant human lung cells, BMC Cancer 8, 229. Julier, C.; Nicolino, M. (2010) Wolcott-Rallison syndrome Orphanet J. Rare Diseases 5 (29) 1-13. http://www.ojrd.com/content/5/1/29
Liu, M. and S. C. Dudley, Jr. (2016) Role for the Unfolded Protein Response in Heart Disease and Cardiac Arrhythmias Int J Mol Sci 17(1) pii: E52. doi: 10.3390/ijms17010052
Lucke-Wold, B. P., et al. (2016). Endoplasmic reticulum stress implicated in chronic traumatic encephalopathy. J Neurosurg 124(3): 687-702.
Kanekura, K.; Suzuki, H.; Aiso, S.; Matsuoka, M. ER Stress and Unfolded Protein Response in Amyotrophic Lateral Sclerosis Molecular Neurobiology (2009), 39(2), 81-89.
Koumenis, C., Naczki, C., Koritzinsky, M., Rastani, S., Diehl, A., Sonenberg, N., Koromilas, A., and Wouters, B. G. (2002). Regulation of protein synthesis by hypoxia via activation of the endoplasmic reticulum kinase PERK and phosphorylation of the translation initiation factor elF2alpha, Mol Cell Biol 22, 7405-16.
Koumenis, C., and Wouters, B. G. (2006). “Translating” tumor hypoxia: unfolded protein response (UPR)-dependent and UPR-independent pathways, Mol Cancer Res 4, 423-36.
Ma, K., Vattem, K. M., and Wek, R. C. (2002). Dimerization and release of molecular chaperone inhibition facilitate activation of eukaryotic initiation factor-2 kinase in response to endoplasmic reticulum stress, J Biol Chem 277, 18728-35.
Ma, Y., and Hendershot, L. M. (2004). The role of the unfolded protein response in tumour development: friend or foe?, Nat Rev Cancer 4, 966-77.
Ma, Y., Lu, Y., Zeng, H., Ron, D., Mo, W., and Neubert, T. A. (2001). Characterization of phosphopeptides from protein digests using matrix-assisted laser desorption/ionization time-of-flight mass spectrometry and nanoelectrospray quadrupole time-of-flight mass spectrometry, Rapid Commun Mass Spectrom 15, 1693-700.
Marciniak, S. J., Garcia-Bonilla, L., Hu, J., Harding, H. P., and Ron, D. (2006). Activation-dependent substrate recruitment by the eukaryotic translation initiation factor 2 kinase PERK, J Cell Biol 172, 201-9.
McAlpine, C. S.; Bowes, A. J.; and Werstuck, G. H. (2010) Diabetes, hyperglycemia and accelerated atherosclerosis: evidence supporting a role for endoplasmic reticulum (ER) stress signaling. Cardiovascular & Hematological Disorders: Drug Targets 10(2), 151-157.
Moreno, J. A; Radford, H.; Peretti, D.; Steinert, J. R.; Verity, N.; Martin, M. G.; Halliday, M.; Morgan, J.; Dinsdale, D.; Ortori, C. A.; Barrett, D. A.; Tsaytler, P.; Bertolotti, A.; Willis, A. E.; Bushell, M.; Mallucci, G. R. (2012) Sustained translational repression by elF2a-P mediates prion neurodegeneration. Nature 485, 507-512.
Moreno, J. A.; Halliday, M.; Molloy, C.; Radford, H.; Verity, N.; Axten, J. M.; Ortori, C. A.; Willis, A. E.; Fischer, P. M.; Barrett, D. A.; Mallucci, G. R. (2013) Oral Treatment Targeting the Unfolded Protein Response Prevents Neurodegeneration and Clinical Disease in Prion-Infected Mice. Science Trans. Med, 5, 206ra138. doi:10.1126/scitranslmed.3006767
Nassif, M.; Matus, S.; Castillo, K.; and Hetz, C. (2010) Amyotrophic Lateral Sclerosis Pathogenesis: A Journey Through the Secretory Pathway Antioxidants & Redox Signaling 13(12), 1955-1989.
Nijholt, D. A., et al. (2012). The unfolded protein response is associated with early tau pathology in the hippocampus of tauopathies. J Pathol 226(5): 693-702.
O'Connor, T.; Sadleir, K. R.; Maus, E.; Velliquette, R. A.; Zhao, J.; Cole, S. L.; Eimer, W. A.; Hitt, B.; Bembinster, L. A.; Lammich, S. Lichtenthaler, S. F., Hébert, S. S., De Strooper, B., Haass, C., Bennett, D. A., Vassar, R. (2008) Phosphorylation of the translation initiation factor elF2α increases BACE1 levels and promotes amyloidogenesis. Neuron, 60(6), 988-1009.
Ohri, S. S.; Maddie, M. A.; Zhao, Y.; Qiu, M. S.; Hetman, M.; Whittemore, S. R. (2011) Attenuating the Endoplasmic Reticulum Stress Response Improves Functional Recovery After Spinal Cord Injury. Glia 59, 1489-1502.
Paschen, W. (2004) Endoplasmic reticulum dysfunction in brain pathology: Critical role of protein synthesis Current Neurovascular Research, 1(2), 173-181.
Prusiner, S. B. (2012) A Unifying Role for Prions in Neurodegenerative Diseases Science 336, 1511-1513.
Romero-Ramirez, L., Cao, H., Nelson, D., Hammond, E., Lee, A. H., Yoshida, H., Mori, K., Glimcher, L. H., Denko, N. C., Giaccia, A. J., et al. (2004). XBP1 is essential for survival under hypoxic conditions and is required for tumor growth, Cancer Res 64, 5943-7.
Ron, D. (2002) Translational control in the endoplasmic reticulum stress response. J. Clin. Invest. 110, 1383-1388.
Rouschop, K. M., van den Beucken, T., Dubois, L., Niessen, H., Bussink, J., Savelkouls, K., Keulers, T., Mujcic, H., Landuyt, W., Voncken, J. W., et al. (2010) The unfolded protein response protects human tumor cells during hypoxia through regulation of the autophagy genes MAP1LC3B and ATG5, J Clin Invest 120, 127-41.
Salminen, A.; Kauppinen, A.; Suuronen, T.; Kaarniranta, K.; Ojala, J. ER stress in Alzheimer's disease: a novel neuronal trigger for inflammation and Alzheimer's pathology. Journal of Neuroinflammation (2009), 6:41.
Shi, Y., Vattem, K. M., Sood, R., An, J., Liang, J., Stramm, L., and Wek, R. C. (1998). Identification and characterization of pancreatic eukaryotic initiation factor 2 alpha-subunit kinase, PEK, involved in translational control, Mol Cell Biol 18, 7499-509.
Sood, R., Porter, A. C., Ma, K., Quilliam, L. A., and Wek, R. C. (2000). Pancreatic eukaryotic initiation factor-2alpha kinase (PEK) homologues in humans, Drosophila melanogaster and Caenorhabditis elegans that mediate translational control in response to endoplasmic reticulum stress, Biochem J 346 Pt 2, 281-93.
Su, Q., Wang, S., Gao, H. Q., Kazemi, S., Harding, H. P., Ron, D., and Koromilas, A. E. (2008). Modulation of the eukaryotic initiation factor 2 alpha-subunit kinase PERK by tyrosine phosphorylation, J Biol Chem 283, 469-75.
Stutzbach, L. D., Xie, S. X., Naj, A. C., Albin, R., Gilman, S., Lee, V. M. Y, Trojanowski, J. Q., Devlin, B., Schellenberg, G. D. (2013) The unfolded protein response is activated in diseaes-affected brain regions in progressive supranuclear palsy and Alzheimer's disease, Acta Neuropath. Comm. 1, 31, http://www.actaneurocomms.org/content/1/1/31
Tabas, I.; Seimon, T.; Timmins, J.; Li, G.; Lim, W. Macrophage apoptosis in advanced atherosclerosis Annals of the New York Academy of Sciences (2009), 1173(S1), E40-E45.
Tajiri, S.; Oyadomari, S.; Yano, S.; Morioka, M.; Gotoh, T.; Hamada, J. I; Ushio, Y.; Mori, M. (2004) Ischemia-induced neuronal cell death is mediated by the endoplasmic reticulum stress pathway involving CHOP. Cell Death and Diff. 11, 403-415.
Unterberger, U.; Hoftberger, R.; Gelpi, E.; Flicker, H.; Budka, H.; Voigtlander, T. (2006) Endoplasmic Reticulum Stress Features Are Prominent in Alzheimer Disease but Not in Prion Diseases In Vivo J. Neuropathol. Exp. Neurol. 65, 348-357.
van Galen, P., et al. (2014). The unfolded protein response governs integrity of the haematopoietic stem-cell pool during stress. Nature 510(7504): 268-272.
Wek, R. C. and D. R. Cavener (2007). Translational control and the unfolded protein response. Antioxid Redox Signal 9(12): 2357-2371.
Walter, P.; Ron, D. (2011) The Unfolded Protein Response: From Stress Pathway to Homeostatic Regulation. Science 334, 1081-1086.
Woehlbier, U.; Hetz, C. Modulating stress responses by the UPRosome: A matter of life and death. Trends Biochem. Sciences 36, 329-337 Zhang, W., Feng, D., Li, Y., Iida, K., McGrath, B., and Cavener, D. R. (2006). PERK EIF2AK3 control of pancreatic beta cell differentiation and proliferation is required for postnatal glucose homeostasis, Cell Metab 4, 491-7.
Zhang, K. and R. J. Kaufman (2006). The unfolded protein response: a stress signaling pathway critical for health and disease. Neurology 66(2 Suppl 1): S102-109 It is an object of the instant invention to provide novel compounds that are inhibitors of PERK.

It is also an object of the present invention to provide pharmaceutical compositions that comprise a pharmaceutical carrier and compounds of Formula (I).

It is also an object of the present invention to provide a method for treating neurodegenerative diseases, cancer, and other diseases/injuries associated with activated unfolded protein response pathways such as: Alzheimer's disease, spinal cord injury, traumatic brain injury, ischemic stroke, stroke, Parkinson disease, diabetes, metabolic syndrome, metabolic disorders, Huntington's disease, Creutzfeldt-Jakob Disease, fatal familial insomnia, Gerstmann-Straussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation that comprises administering novel inhibitors of PERK activity.

### SUMMARY OF THE INVENTION

The invention is directed to substituted isoquinoline derivatives the uses thereof.

Specifically, the invention is directed to compounds according to Formula I and the use of compounds of Formula (I) in treating disease states:

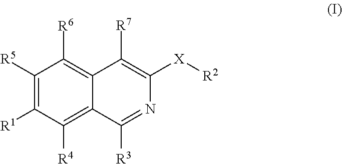

wherein R¹, R², R³, R⁴, R⁵, R⁶, R⁷ and X are as defined below; or a salt thereof including a pharmaceutically acceptable salt thereof.

The present invention also relates to the discovery that the compounds of Formula (I) are active as inhibitors of PERK.

This invention also relates to a method of treating cancer, which comprises administering to a subject in need thereof an effective amount of a PERK inhibiting compound of Formula (I).

This invention also relates to a method of treating Alzheimer's disease, which comprises administering to a subject in need thereof an effective amount of a PERK inhibiting compound of Formula (I).

This invention also relates to a method of treating Parkinson's disease, which comprises administering to a subject in need thereof an effective amount of a PERK inhibiting compound of Formula (I).

This invention also relates to a method of treating amyotrophic lateral sclerosis, which comprises administering to a subject in need thereof an effective amount of a PERK inhibiting compound of Formula (I).

This invention also relates to a method of treating Huntington's disease, which comprises administering to a subject in need thereof an effective amount of a PERK inhibiting compound of Formula (I).

This invention also relates to a method of treating Creutzfeldt-Jakob Disease, which comprises administering to a subject in need thereof an effective amount of a PERK inhibiting compound of Formula (I).

This invention also relates to a method of treating progressive supranuclear palsy (PSP), which comprises administering to a subject in need thereof an effective amount of a PERK inhibiting compound of Formula (I).

This invention also relates to a method of treating dementia, which comprises administering to a subject in need thereof an effective amount of a PERK inhibiting compound of Formula (I).

This invention also relates to a method of treating spinal cord injury, which comprises administering to a subject in need thereof an effective amount of a PERK inhibiting compound of Formula (I).

This invention also relates to a method of treating traumatic brain injury, which comprises administering to a subject in need thereof an effective amount of a PERK inhibiting compound of Formula (I).

This invention also relates to a method of treating ischemic stroke, which comprises administering to a subject in need thereof an effective amount of a PERK inhibiting compound of Formula (I).

This invention also relates to a method of treating diabetes, which comprises administering to a subject in need thereof an effective amount of a PERK inhibiting compound of Formula (I).

This invention also relates to a method of treating a disease state selected from: myocardial infarction, cardiovascular disease, atherosclerosis, ocular diseases, and arrhythmias, which comprises administering to a subject in need thereof an effective amount of a PERK inhibiting compound of Formula (I).

This invention also relates to a method of using the compounds of Formula (I) in organ transplantation and in the transportation of organs for transplantation.

In a further aspect of the invention there is provided novel processes and novel intermediates useful in preparing the presently invented PERK inhibiting compounds.

Included in the present invention are pharmaceutical compositions that comprise a pharmaceutical carrier and compounds useful in the methods of the invention.

Also included in the present invention are methods of co-administering the presently invented PERK inhibiting compounds with further active ingredients.

The invention also relates to a compound of Formula (I) or a pharmaceutically acceptable salt thereof for use in therapy.

The invention also relates to a compound of Formula (I) or a pharmaceutically acceptable salt thereof for use in the treatment of Alzheimer's disease.

The invention also relates to a compound of Formula (I) or a pharmaceutically acceptable salt thereof for use in the treatment of Parkinson's disease.

The invention also relates to a compound of Formula (I) or a pharmaceutically acceptable salt thereof for use in the treatment of amyotrophic lateral sclerosis.

The invention also relates to a compound of Formula (I) or a pharmaceutically acceptable salt thereof for use in the treatment of Huntington's disease.

The invention also relates to a compound of Formula (I) or a pharmaceutically acceptable salt thereof for use in the treatment of Creutzfeldt-Jakob Disease.

The invention also relates to a compound of Formula (I) or a pharmaceutically acceptable salt thereof for use in the treatment of progressive supranuclear palsy (PSP).

The invention also relates to a compound of Formula (I) or a pharmaceutically acceptable salt thereof for use in the treatment of dementia.

The invention also relates to a compound of Formula (I) or a pharmaceutically acceptable salt thereof for use in the treatment of spinal cord injury.

The invention also relates to the use of a compound of Formula (I) or a pharmaceutically acceptable salt thereof in the preparation of a medicament for the treatment of traumatic brain injury.

The invention also relates to the use of a compound of Formula (I) or a pharmaceutically acceptable salt thereof in the preparation of a medicament for the treatment of diabetes.

The invention also relates to the use of a compound of Formula (I) or a pharmaceutically acceptable salt thereof in the preparation of a medicament for the treatment of a disease state selected from: myocardial infarction, cardiovascular disease, atherosclerosis, ocular diseases, and arrhythmias.

The invention also relates to the use of a compound of Formula (I) or a pharmaceutically acceptable salt thereof in the preparation of a medicament for the treatment of chronic traumatic encephalopathy (CTE).

The invention also relates to the use of a compound of Formula (I) or a pharmaceutically acceptable salt thereof in the preparation of a medicament for use in organ transplantation and in the transportation of organs for transplantation.

Included in the present invention are pharmaceutical compositions that comprise a pharmaceutical carrier and a compound of Formula (I) or a pharmaceutically acceptable salt thereof.

The invention also relates to a pharmaceutical composition as defined above for use in therapy.

One embodiment of this invention provides a combination comprising:

a) a compound of Formula (I) or a pharmaceutically acceptable salt thereof; and

b) an ATF-4 modulating compound.

### DETAILED DESCRIPTION OF THE INVENTION

This invention relates to compounds of Formula (I) and to the use of compounds of Formula (I) in the methods of the invention:

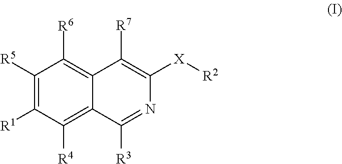

wherein:
R¹ is selected from:
   bicycloheteroaryl,
   substituted bicycloheteroaryl,
   heteroaryl, and
   substituted heteroaryl,
   where said substituted bicycloheteroaryl and said substituted heteroaryl are
   substituted with from one to five substituents independently selected from:
      fluoro,
      chloro,
      bromo,
      iodo,
      C₁₋₆alkyl,
      C₁₋₆alkyl substituted with from 1 to 5 substituents independently selected from: fluoro, chloro, bromo, iodo, C₁₋₄alkyloxy, —OH, C₁₋₄alkyl, cycloalkyl, —COOH, —CF₃, —NO₂, —NH₂ and —CN,
      —OH,
      hydroxyC₁₋₆alkyl,
      —COOH,
      tetrazole,
      cycloalkyl,
      oxo,
      —OC₁₋₆alkyl,
      —CF₃,
      —CF₂H,
      —CFH₂,
      C₁₋₆alkylOC₁₋₄alkyl,
      —CONH₂,
      —CON(H)C₁₋₃alkyl,
      diC₁₋₄alkylaminoC₁₋₄alkyl,
      aminoC₁₋₆alkyl,
      —CN,
      heterocycloalkyl,
      heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, oxo, —NO₂, —NH₂ and —CN,
      —NO₂,
      —NH₂,
      —N(H)C₁₋₃alkyl, and
      —N(C₁₋₃alkyl)₂,
R² is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN,
   heteroaryl,
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   bicycloheteroaryl,
   bicycloheteroaryl substituted with from one to five substituents
      independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —CHF₂, —OCF₃, —CN, and cycloalkyl;
R³, R⁴, R⁵, and R⁶ are each independently selected from hydrogen, fluoro, chloro, bromo, iodo, —CF₃, and —CH₃; and
R⁷ is selected from: hydrogen, C₁₋₆alkyl, cycloalkyl, aminoC₁₋₆alkyl, —CF₃, —CH₃, fluoro, chloro, bromo and iodo; and
X is O, S, C(═O), NR¹⁰⁰, CR²⁰⁰R³⁰⁰,
   where R¹⁰⁰ is selected from hydrogen, C₁₋₆alkyl;
   R²⁰⁰ and R³⁰⁰ are independently selected from hydrogen, —CH₃,
   —CF₃, —OH, —NH₂,
   or R²⁰⁰ and R³⁰⁰ taken together with the carbon atoms to which they are attached represent a 3 or 4 member cycloalkyl;
      and salts thereof.

This invention also relates to pharmaceutically acceptable salts of the compounds of Formula (I).

Suitably, in the compounds of Formula (I), X is CR²⁰⁰R³⁰⁰, where R²⁰⁰ and R³⁰⁰ are independently selected from selected from: hydrogen and —CH₃.

Suitably, in the compounds of Formula (I), X is C(═O).

Suitably, in the compounds of Formula (I), R¹ is a substituted pyrrolo[2,3-d]pyrimidine.

Suitably, in the compounds of Formula (I), R¹ is a substituted pyrazolo[3,4-d]pyrimidine.

Suitably, in the compounds of Formula (I), R¹ is a substituted pyrrolo[3,2-c]pyridine.

Suitably, in the compounds of Formula (I), R² is selected from:
aryl, and
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN.

Suitably, in the compounds of Formula (I), R⁷ is hydrogen.

Suitably, in the compounds of Formula (I), R³, R⁵, and R⁶ are hydrogen.

Suitably, in the compounds of Formula (I), R⁴ is fluoro.

Included in the compounds of the invention and used in the methods of the invention are compounds of Formula (II):

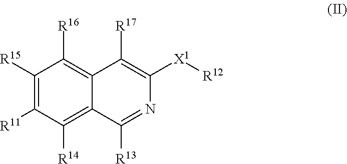

wherein:
R¹¹ is selected from:
   bicycloheteroaryl,
   substituted bicycloheteroaryl,
   heteroaryl, and
   substituted heteroaryl,
   where said substituted bicycloheteroaryl and said substituted heteroaryl are substituted with from one to five substituents independently selected from:
      fluoro,
      chloro,
      bromo,
      iodo,
      C₁₋₆alkyl,
      C₁₋₆alkyl substituted with from 1 to 5 substituents independently selected from: fluoro, chloro, bromo, iodo, C₁₋₄alkyloxy, —OH, C₁₋₄alkyl, cycloalkyl, —COOH, —CF₃, —NO₂, —NH₂ and —CN,
      —OH,
      hydroxyC₁₋₆alkyl,
      —COOH,
      tetrazole,
      cycloalkyl,
      oxo,
      —OC₁₋₆alkyl,
      —CF₃,
      —CF₂H,
      —CFH₂,
      —C₁₋₆alkylOC₁₋₄alkyl,
      —CONH₂,
      —CON(H)C₁₋₃alkyl,
      diC₁₋₄alkylaminoC₁₋₄alkyl,
      aminoC₁₋₆alkyl,
      —CN,
      heterocycloalkyl,
      heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, oxo, —NO₂, —NH₂ and —CN,
      —NO₂,
      —NH₂,
      —N(H)C₁₋₃alkyl, and
      —N(C₁₋₃alkyl)₂,
R¹² is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN,
   heteroaryl,
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   bicycloheteroaryl,
   bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —CHF₂, —OCF₃, —CN, and cycloalkyl;
R¹³, R¹⁴, R¹⁵, and R¹⁶ are each independently selected from hydrogen, fluoro, chloro, bromo, iodo, —CF₃, and —CH₃; and
R¹⁷ is selected from: hydrogen, C₁₋₆alkyl, cycloalkyl, aminoC₁₋₆alkyl, —CF₃, —CH₃, fluoro, chloro, bromo and iodo; and
X¹ is O, S, C(═O), CR²⁵⁰R³⁵⁰,
   R²⁵⁰ and R³⁵⁰ are independently selected from hydrogen, —CH₃, —CF₃,
   —OH, NH₂,
   or R²⁵⁰ and R³⁵⁰ taken together with the carbon atoms to which they are attached represent a 3 or 4 member cycloalkyl;
      and salts thereof.

This invention also relates to pharmaceutically acceptable salts of the compounds of Formula (II).

Suitably, in the compounds of Formula (II), X¹ is CR²⁵⁰R³⁵⁰, where R²⁵⁰ and R³⁵⁰ are independently selected from selected from: hydrogen and —CH₃.

Suitably, in the compounds of Formula (II), X¹ is C(═O).

Suitably, in the compounds of Formula (II), R¹¹ is a substituted pyrrolo[2,3-d]pyrimidine.

Suitably, in the compounds of Formula (II), R¹¹ is a substituted pyrazolo[3,4-d]pyrimidine.

Suitably, in the compounds of Formula (II), R¹¹ is a substituted pyrrolo[3,2-c]pyridine.

Suitably, in the compounds of Formula (II), R¹² is selected from:
aryl, and
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN.

Suitably, in the compounds of Formula (II), R¹⁷ is hydrogen.

Suitably, in the compounds of Formula (II), R¹³, R¹⁵, and R¹⁶ are hydrogen.

Suitably, in the compounds of Formula (II), R¹⁴ is fluoro.

Included in the compounds of the invention and used in the methods of the invention are compounds of Formula (III):

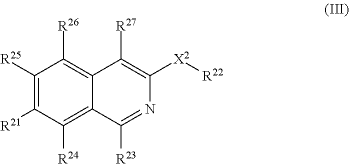

wherein:
R²¹ is selected from:
   bicycloheteroaryl, and
   substituted bicycloheteroaryl,
   where said substituted bicycloheteroaryl is substituted with from one to five
   substituents independently selected from:
      fluoro,
      chloro,
      bromo,
      iodo,
      C₁₋₆alkyl,
      C₁₋₆alkyl substituted with from 1 to 5 substituents independently selected from: fluoro, chloro, bromo, iodo, C₁₋₄alkyloxy, —OH, C₁₋₄alkyl, cycloalkyl, —COOH, —CF₃, —NO₂, —NH₂ and —CN,
      —OH,
      hydroxyC₁₋₆alkyl,
      —COOH,
      tetrazole,
      cycloalkyl,
      oxo,
      —OC₁₋₆alkyl,
      —CF₃,
      —CF₂H,
      —CFH₂,
      —C₁₋₆alkylOC₁₋₄alkyl,
      —CONH₂,
      —CON(H)C₁₋₃alkyl,
      diC₁₋₄alkylaminoC₁₋₄alkyl,
      aminoC₁₋₆alkyl,
      —CN,
      heterocycloalkyl,
      heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, oxo, —NO₂, —NH₂ and —CN,
      —NO₂,
      —NH₂,
      —N(H)C₁₋₃alkyl, and
      —N(C₁₋₃alkyl)₂,
R²² is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN,
   heteroaryl,
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   bicycloheteroaryl,
   bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —CHF₂, —OCF₃, —CN, and cycloalkyl;
R²³, R²⁴, R²⁵, and R²⁶ are each independently selected from hydrogen, fluoro, chloro, bromo, iodo, —CF₃, and —CH₃; and
R²⁷ is selected from: hydrogen, C₁₋₆alkyl, cycloalkyl, —CF₃, —CH₃, fluoro, chloro, bromo and iodo; and
X² is O, S, C(═O), CR²⁶⁰R³⁶⁰,
   R²⁶⁰ and R³⁶⁰ are independently selected from hydrogen, —CH₃, —CF₃,
   —OH, —NH₂,
   or R²⁶⁰ and R³⁶⁰ taken together with the carbon atoms to which they are
   attached represent a 3 or 4 member cycloalkyl;
      and salts thereof.

This invention also relates to pharmaceutically acceptable salts of the compounds of Formula (III).

Suitably, in the compounds of Formula (III), X² is CR²⁶⁰R³⁶⁰, where R²⁶⁰ and R³⁶⁰ are independently selected from selected from: hydrogen and —CH₃.

Suitably, in the compounds of Formula (III), X² is C(═O).

Suitably, in the compounds of Formula (III), R²¹ is a substituted pyrrolo[2,3-d]pyrimidine.

Suitably, in the compounds of Formula (III), R²¹ is a substituted pyrazolo[3,4-d]pyrimidine.

Suitably, in the compounds of Formula (III), R²¹ is a substituted pyrrolo[3,2-c]pyridine.

Suitably, in the compounds of Formula (III), R²² is selected from:
aryl, and
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN.

Suitably, in the compounds of Formula (III), R²⁷ is hydrogen.

Suitably, in the compounds of Formula (III), R²³, R²⁵, and R²⁶ are hydrogen.

Suitably, in the compounds of Formula (III), R²⁴ is fluoro.

Included in the compounds of the invention and used in the methods of the invention are compounds of Formula (IV):

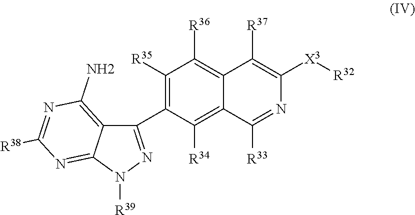

wherein:
R³² is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN,
   heteroaryl,
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   bicycloheteroaryl,
   bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —CHF₂, —OCF₃, —CN, and cycloalkyl;
R³³, R³⁴, R³⁵, and R³⁶ are each independently selected from hydrogen, fluoro, chloro, bromo, iodo, —CF₃, and —CH₃; and
R³⁷ is selected from: hydrogen, C₁₋₆alkyl, cycloalkyl, —CF₃, —CH₃, fluoro, chloro, bromo and iodo;
R³⁸ is selected from: hydrogen and —CH₃; and
R³⁹ is selected from:
   hydrogen,
   cycloalkyl,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
X³ is O, S, C(═O), CR²⁷⁰R³⁷⁰,
   R²⁷⁰ and R³⁷⁰ are independently selected from hydrogen, —CH₃, —CF₃, —OH, —NH₂,
   or R²⁷⁰ and R³⁷⁰ taken together with the carbon atoms to which they are attached represent a 3 or 4 member cycloalkyl;
      and salts thereof.

This invention also relates to pharmaceutically acceptable salts of the compounds of Formula (IV).

Suitably, in the compounds of Formula (IV), X³ is CR²⁷⁰R³⁷⁰, where R²⁷⁰ and R³⁷⁰ are independently selected from selected from: hydrogen and —CH₃.

Suitably, in the compounds of Formula (IV), X³ is C(═O).

Suitably, in the compounds of Formula (IV), R³² is selected from:
aryl, and
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN.

Suitably, in the compounds of Formula (IV), R³⁷ is hydrogen.

Suitably, in the compounds of Formula (IV), R³³, R³⁵, and R³⁶ are hydrogen.

Suitably, in the compounds of Formula (IV), R³⁴ is fluoro.

Included in the compounds of the invention and used in the methods of the invention are compounds of Formula (V):

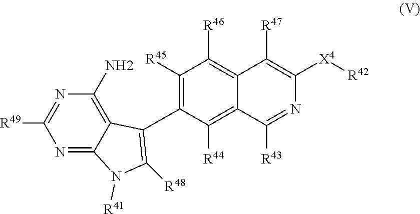

wherein:
R⁴¹ is selected from:
   hydrogen,
   cycloalkyl,
   heterocycloalkyl,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R⁴² is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN,
   heteroaryl,
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   bicycloheteroaryl,
   bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —CHF₂, —OCF₃, —CN, and cycloalkyl;
R⁴³, R⁴⁴, R⁴⁵, and R⁴⁶ are each independently selected from hydrogen, fluoro, chloro, bromo, iodo, —CF₃, and —CH₃; and
R⁴⁷ is selected from: hydrogen, C₁₋₆alkyl, cycloalkyl, —CF₃, —CH₃, fluoro, chloro, bromo and iodo;
R⁴⁸ is selected from: hydrogen and C₁₋₆alkyl;
R⁴⁹ is selected from: hydrogen and —CH₃; and
X⁴ is O, S, C(═O), CR²⁸⁰R³⁸⁰,
   R²⁸⁰ and R³⁸⁰ are independently selected from hydrogen, —CH₃, —CF₃, —OH, —NH₂,
   or R²⁸⁰ and R³⁸⁰ taken together with the carbon atoms to which they are attached represent a 3 or 4 member cycloalkyl;
      and salts thereof.

This invention also relates to pharmaceutically acceptable salts of the compounds of Formula (V).

Suitably, in the compounds of Formula (V), X⁴ is CR²⁸⁰R³⁸⁰, where R²⁸⁰ and R³⁸⁰ are independently selected from selected from: hydrogen and —CH₃.

Suitably, in the compounds of Formula (V), X⁴ is C(═O).

Suitably, in the compounds of Formula (V), R⁴² is selected from:
aryl, and
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN.

Suitably, in the compounds of Formula (V), R⁴⁷ is hydrogen.

Suitably, in the compounds of Formula (V), R⁴³, R⁴⁵, and R⁴⁶ are hydrogen.

Suitably, in the compounds of Formula (IV), R⁴⁴ is fluoro.

Included in the novel compounds of the invention are:
5-(3-Benzylisoquinolin-7-yl)-7-methyl-7H-pyrrolo[2,3-d]pyrimidin-4-amine;
5-(3-(3,5-Dimethylbenzyl)isoquinolin-7-yl)-7-methyl-7H-pyrrolo[2,3-d]pyrimidin-4-amine;
5-(3-Benzyl-8-fluoroisoquinolin-7-yl)-7-methyl-7H-pyrrolo[2,3-d]pyrimidin-4-amine;
5-(3-(3,5-Difluorobenzyl)-8-fluoroisoquinolin-7-yl)-7-methyl-7H-pyrrolo[2,3-d]pyrimidin-4-amine;
7-cyclopropyl-5-(3-(2,3-difluorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-ethyl-7H-pyrrolo[2,3-d]pyrimidin-4-amine;
(7-(4-amino-7-cyclopropyl-7H-pyrrolo[2,3-d]pyrimidin-5-yl)-8-fluoroisoquinolin-3-yl)(3,5-difluorophenyl)methanol;
7-cyclopropyl-5-(3-(3,5-difluorobenzyl)-5-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2,2-difluorocyclopropyl)-7H-pyrrolo[2,3-d]pyrimidin-4-amine;
3-(3-(3,5-difluorobenzyl)-8-fluoroisoquinolin-7-yl)-7-fluoro-1-methyl-1H-pyrrolo[3,2-c]pyridin-4-amine;
5-(3-(3,5-difluorobenzyl)-8-fluoroisoquinolin-7-yl)-7-(oxetan-3-yl)-7H-pyrrolo[2,3-d]pyrimidin-4-amine;
7-cyclopropyl-5-(3-(3,5-difluorobenzyl)-8-fluoro-4-methylisoquinolin-7-yl)-7H-pyrrolo[2,3-d]pyrimidin-4-amine;
(7-(4-amino-7-methyl-7H-pyrrolo[2,3-d]pyrimidin-5-yl)isoquinolin-3-yl)(3,5-dimethylphenyl)methanone;
5-(3-(3,4-difluorobenzyl)-8-fluoroisoquinolin-7-yl)-7-methyl-7H-pyrrolo[2,3-d]pyrimidin-4-amine;
5-(3-(2,5-difluorobenzyl)-8-fluoroisoquinolin-7-yl)-7-methyl-7H-pyrrolo[2,3-d]pyrimidin-4-amine;
5-(8-fluoro-3-(3-fluoro-5-(trifluoromethyl)benzyl)isoquinolin-7-yl)-7-methyl-7H-pyrrolo[2,3-d]pyrimidin-4-amine;
5-(8-fluoro-3-(3-(trifluoromethyl)benzyl)isoquinolin-7-yl)-7-methyl-7H-pyrrolo[2,3-d]pyrimidin-4-amine;
7-cyclopropyl-5-(8-fluoro-3-(3-fluorobenzyl)isoquinolin-7-yl)-7H-pyrrolo[2,3-d]pyrimidin-4-amine;
7-cyclopropyl-5-(8-fluoro-3-(4-fluorobenzyl)isoquinolin-7-yl)-7H-pyrrolo[2,3-d]pyrimidin-4-amine;
7-cyclopropyl-5-(3-(2,5-dimethylbenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2,2,2-trifluoroethyl)-7H-pyrrolo[2,3-d]pyrimidin-4-amine;
5-(3-(3,5-difluorobenzyl)-8-fluoroisoquinolin-7-yl)-7-isopropyl-7H-pyrrolo[2,3-d]pyrimidin-4-amine;
5-(3-(3,5-difluorobenzyl)-8-fluoroisoquinolin-7-yl)-2,7-dimethyl-7H-pyrrolo[2,3-d]pyrimidin-4-amine;
7-cyclopropyl-5-(3-(3,5-difluorobenzyl)-8-fluoroisoquinolin-7-yl)-7H-pyrrolo[2,3-d]pyrimidin-4-amine;
3-(3-(3,5-difluorobenzyl)-8-fluoroisoquinolin-7-yl)-1-methyl-1H-pyrrolo[3,2-c]pyridin-4-amine;
7-cyclopropyl-5-(3-(3,5-difluorobenzyl)-8-fluoroisoquinolin-7-yl)-2-methyl-7H-pyrrolo[2,3-d]pyrimidin-4-amine;
1-cyclopropyl-3-(3-(3,5-difluorobenzyl)-8-fluoroisoquinolin-7-yl)-1H-pyrazolo[3,4-d]pyrimidin-4-amine;
7-cyclopropyl-5-(3-((3,5-difluorophenyl)(methoxy) methyl)-8-fluoroisoquinolin-7-yl)-7H-pyrrolo[2,3-d]pyrimidin-4-amine;
7-(2-(2-aminoethoxy)ethyl)-5-(3-(3,5-difluorobenzyl)-8-fluoroisoquinolin-7-yl)-7H-pyrrolo[2,3-d]pyrimidin-4-amine;
7-(2-aminoethyl)-5-(3-(3,5-difluorobenzyl)-8-fluoroisoquinolin-7-yl)-7H-pyrrolo[2,3-d]pyrimidin-4-amine;
7-cyclopropyl-5-(3-(3-ethynyl-5-fluorobenzyl)-8-fluoroisoquinolin-7-yl)-7H-pyrrolo[2,3-d]pyrimidin-4-amine;
7-cyclopropyl-5-(3-(2,5-difluorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(1-methylpiperidin-4-yl)-7H-pyrrolo[2,3-d]pyrimidin-4-amine;
5-(3-(3,5-difluorobenzyl)-8-fluoroisoquinolin-7-yl)-7-(2-morpholinoethyl)-7H-pyrrolo[2,3-d]pyrimidin-4-amine;
5-(3-(5-chloro-2-methylbenzyl)-8-fluoroisoquinolin-7-yl)-7-cyclopropyl-7H-pyrrolo[2,3-d]pyrimidin-4-amine;
7-cyclopropyl-5-(8-fluoro-3-(2-methylbenzyl)isoquinolin-7-yl)-7H-pyrrolo[2,3-d]pyrimidin-4-amine;
5-(3-(3,5-difluorobenzyl)-8-fluoroisoquinolin-7-yl)-7-(1-methylazetidin-3-yl)-7H-pyrrolo[2,3-d]pyrimidin-4-amine;
7-cyclopropyl-5-(3-(1-(3,5-difluorophenyl)ethyl)-8-fluoroisoquinolin-7-yl)-7H-pyrrolo[2,3-d]pyrimidin-4-amine;
7-cyclopropyl-5-(8-fluoro-3-(2-fluoro-5-(trifluoromethyl)benzyl)isoquinolin-7-yl)-7H-pyrrolo[2,3-d]pyrimidin-4-amine;
5-(3-(3,5-difluorobenzyl)isoquinolin-7-yl)-7-methyl-7H-pyrrolo[2,3-d]pyrimidin-4-amine;
5-(3-(3-chlorobenzyl)-8-fluoroisoquinolin-7-yl)-7-cyclopropyl-7H-pyrrolo[2,3-d]pyrimidin-4-amine;
5-(3-(2-chlorobenzyl)-8-fluoroisoquinolin-7-yl)-7-cyclopropyl-7H-pyrrolo[2,3-d]pyrimidin-4-amine;
7-cyclopropyl-5-(8-fluoro-3-(3-fluoro-5-methylbenzyl)isoquinolin-7-yl)-7H-pyrrolo[2,3-d]pyrimidin-4-amine;
7-cyclopropyl-5-(3-(3,5-dichlorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2-(dimethylamino)ethyl)-7H-pyrrolo[2,3-d]pyrimidin-4-amine;
5-(8-fluoro-3-(3-fluorobenzyl)isoquinolin-7-yl)-7-methyl-7H-pyrrolo[2,3-d]pyrimidin-4-amine;
5-(3-(3-chlorobenzyl)-8-fluoroisoquinolin-7-methyl-7H-pyrrolo[2,3-d]pyrimidin-4-amine;
7-cyclobutyl-5-(3-(3,5-difluorobenzyl)-8-fluoroisoquinolin-7-yl)-7H-pyrrolo[2,3-d]pyrimidin-4-amine;
5-(3-(3-chloro-2-fluorobenzyl)-8-fluoroisoquinolin-7-yl)-7-cyclopropyl-7H-pyrrolo[2,3-d]pyrimidin-4-amine;
7-cyclopropyl-5-(3-(2,3-difluorobenzyl)-8-fluoroisoquinolin-7-yl)-7H-pyrrolo[2,3-d]pyrimidin-4-amine;
7-cyclopropyl-5-(8-fluoro-3-((5-fluoropyrid in-3-yl)methyl)isoquinolin-7-yl)-7H-pyrrolo[2,3-d]pyrimidin-4-amine;
7-(cyclopropylmethyl)-5-(3-(3,5-difluorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2-methoxyethyl)-7H-pyrrolo[[2,3-d]pyrimidin-4-amine;
5-(3-(3,5-difluorobenzyl)-8-fluoroisoquinolin-7-yl)-7-((3-methyloxetan-3-yl)methyl)-7H-pyrrolo[2,3-d]pyrimidin-4-amine;
7-cyclopropyl-5-(3-(3,5-difluorobenzyl)-8-fluoroisoquinolin-7-yl)-6-methyl-7H-pyrrolo[2,3-d]pyrimidin-4-amine;
5-(3-(3,5-difluorobenzyl)-8-fluoroisoquinolin-7-yl)pyrrolo[2,1-f][1,2,4]triazin-4-amine;
5-(3-(3,5-difluorobenzyl)-8-fluoroisoquinolin-7-yl)-7-ethyl-6-methyl-7H-pyrrolo[2,3-d]pyrimidin-4-amine; and
5-(3-(3-chloro-5-fluorobenzyl)-8-fluoroisoquinolin-7-yl)-7-cyclopropyl-7H-pyrrolo[2,3-d]pyrimidin-4-amine;
   and salts thereof including pharmaceutically acceptable salts thereof.

The skilled artisan will appreciate that salts, including pharmaceutically acceptable salts, of the compounds according to Formula (I) may be prepared. Indeed, in certain embodiments of the invention, salts including pharmaceutically-acceptable salts of the compounds according to Formula (I) may be preferred over the respective free or unsalted compound. Accordingly, the invention is further directed to salts, including pharmaceutically-acceptable salts, of the compounds according to Formula (I).

The salts, including pharmaceutically acceptable salts, of the compounds of the invention are readily prepared by those of skill in the art.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (I), or in any chemical structure illustrated herein, if not specified the structure is intended to encompass all individual stereoisomers and all mixtures thereof. Thus, compounds according to Formula (I) containing one or more chiral centers may be used as racemic mixtures, enantiomerically enriched mixtures, or as enantiomerically pure individual stereoisomers.

The compounds according to Formula (I) may also contain double bonds or other centers of geometric asymmetry. Where the stereochemistry of a center of geometric asymmetry present in Formula (I), or in any chemical structure illustrated herein, is not specified, the structure is intended to encompass the trans (E) geometric isomer, the cis (Z) geometric isomer, and all mixtures thereof. Likewise, all tautomeric forms are also included in Formula (I) whether such tautomers exist in equilibrium or predominately in one form.

The compounds of Formula (I) or salts, including pharmaceutically acceptable salts, thereof may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates wherein water is the solvent that is incorporated into the crystalline lattice are typically referred to as “hydrates.” Hydrates include stoichiometric hydrates as well as compositions containing vaiable amounts of water.

The skilled artisan will further appreciate that certain compounds of Formula (I) or salts, including pharmaceutically acceptable salts thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions. The invention includes all such polymorphs.

### Definitions

“Alkyl” refers to a hydrocarbon chain having the specified number of “member atoms”. For example, C₁-C₆ alkyl refers to an alkyl group having from 1 to 6 member atoms. Alkyl groups may be saturated, unsaturated, straight or branched. Representative branched alkyl groups have one, two, or three branches. Alkyl includes, but is not limited to: methyl, ethyl, ethylene, alkynyl (such as ethynyl), propyl (n-propyl and isopropyl), butene, butyl (n-butyl, isobutyl, and t-butyl), pentyl and hexyl.
“Alkoxy” refers to an —O-alkyl group wherein “alkyl” is as defined herein. For example, C₁-C₄alkoxy refers to an alkoxy group having from 1 to 4 member atoms. Representative branched alkoxy groups have one, two, or three branches. Examples of such groups include methoxy, ethoxy, propoxy, and butoxy.
“Aryl” refers to an aromatic hydrocarbon ring. Aryl groups are monocyclic, bicyclic, and tricyclic ring systems having a total of five to fourteen ring member atoms, wherein at least one ring system is aromatic and wherein each ring in the system contains 3 to 7 member atoms, such as phenyl, naphthalene, tetrahydronaphthalene and biphenyl. Suitably aryl is phenyl.
“Bicycloheteroaryl” refers to two fused aromatic rings containing from 1 to 6 heteroatoms as member atoms. Bicycloheteroaryl groups containing more than one heteroatom may contain different heteroatoms. Bicycloheteroaryl rings have from 6 to 11 member atoms.
Bicycloheteroaryl includes: 1H-pyrrolo[3,2-c]pyridine, 1H-pyrazolo[4,3-c]pyridine, 1H-pyrazolo[3,4-d]pyrimidine, 1H-pyrrolo[2,3-d]pyrimidine, 7H-pyrrolo[2,3-d]pyrimidine, thieno[3,2-c]pyridine, thieno[2,3-d]pyrimidine, furo[2,3-c]pyridine, furo[2,3-d]pyrimidine, pyrrolo[2,1-f][1,2,4]triazin-4-amine, indolyl, isoindolyl, indolizinyl, indazolyl, purinyl, quinolinyl, isoquinolinyl, quinoxalinyl, quinazolinyl, pteridinyl, cinnolinyl, azabenzimidazolyl, tetrahydrobenzimidazolyl, benzimidazolyl, benopyranyl, benzoxazolyl, benzofuranyl, isobenzofuranyl, benzothiazolyl, benzothienyl, imidazo[4.5-c]pyridine, imidazo[4.5-b]pyridine, furopyridinyl and napthyridinyl.
Suitably “Bicycloheteroaryl” includes: 1H-pyrazolo[3,4-d]pyrimidine, 1H-pyrrolo[2,3-d]pyrimidine, 7H-pyrrolo[2,3-d]pyrimidine, thieno[3,2-c]pyridine, thieno[2,3-d]pyrimidine, furo[2,3-c]pyridine, indolyl, isoindolyl, indolizinyl, indazolyl, purinyl, quinolinyl, isoquinolinyl, quinoxalinyl, quinazolinyl, pteridinyl, cinnolinyl, azabenzimidazolyl, tetrahydrobenzimidazolyl, benzimidazolyl, benopyranyl, benzoxazolyl, benzofuranyl, isobenzofuranyl, benzothiazolyl, benzothienyl, imidazo[4.5-c]pyridine, imidazo[4.5-b]pyridine, furopyridinyl and napthyridinyl. Suitably 1H-pyrazolo[3,4-d]pyrimidine, 1H-pyrrolo[2,3-d]pyrimidine, thieno[3,2-c]pyridine, thieno[2,3-d]pyrimidine, indazolyl, quinolinyl, quinazolinyl or benzothiazolyl. Suitably 1H-pyrazolo[3,4-d]pyrimidine, thieno[2,3-d]pyrimidine or 1H-pyrrolo[2,3-d]pyrimidine. Suitably 1H-pyrrolo[2,3-d]pyrimidine.
“Cycloalkyl”, unless otherwise defined, refers to a saturated or unsaturated non aromatic hydrocarbon ring having from three to seven carbon atoms. Cycloalkyl groups are monocyclic ring systems. For example, C₃-C₇ cycloalkyl refers to a cycloalkyl group having from 3 to 7 member atoms. Examples of cycloalkyl as used herein include: cyclopropyl, cyclobutyl, cyclopentyl, cyclohexyl, cyclobutenyl, cyclopentenyl, cyclohexenyl and cycloheptyl.
“Halo” refers to the halogen radicals fluoro, chloro, bromo, and iodo.
“Heteroaryl” refers to a monocyclic aromatic 4 to 8 member ring containing from 1 to 7 carbon atoms and containing from 1 to 4 heteroatoms, provided that when the number of carbon atoms is 3, the aromatic ring contains at least two heteroatoms. Heteroaryl groups containing more than one heteroatom may contain different heteroatoms. Heteroaryl includes: pyrrolyl, pyrazolyl, imidazolyl, oxazolyl, isoxazolyl, thiazolyl, isothiazolyl, furanyl, furazanyl, thienyl, triazolyl, pyridinyl, pyrimidinyl, pyridazinyl, pyrazinyl, triazinyl, tetrazinyl. Suitably, “heteroaryl” includes: pyrazole, pyrrole, isoxazole, pyridine, pyrimidine, pyridazine, and imidazole.
“Heterocycloalkyl” refers to a saturated or unsaturated non-aromatic ring containing 4 to 12 member atoms, of which 1 to 11 are carbon atoms and from 1 to 6 are heteroatoms. Heterocycloalkyl groups containing more than one heteroatom may contain different heteroatoms. Heterocycloalkyl groups are monocyclic ring systems or a monocyclic ring fused with an aryl ring or to a heteroaryl ring having from 3 to 6 member atoms. Heterocycloalkyl includes: pyrrolidinyl, tetrahydrofuranyl, dihydrofuranyl, pyranyl, tetrahydropyranyl, dihydropyranyl, tetrahydrothienyl, pyrazolidinyl, oxazolidinyl, oxetanyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, 1,3oxazolidin-2-one, hexahydro-1H-azepin, 4,5,6,7,tetrahydro-1H-benzimidazol, piperidinyl, 1,2,3,6-tetrahydro-pyridinyl and azetidinyl.
“Heteroatom” refers to a nitrogen, sulphur or oxygen atom.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

### Ac (Acetyl);

Ac₂O (Acetic anhydride);

### ACN (Acetonitrile);

### AIBN (Azobis(isobutyronitrile));

BINAP (2,2′-Bis(diphenylphosphino)-1,1′-binaphthyl);
BMS (Borane-dimethyl sulphide complex);

### Bn (Benzyl);

### Boc (Tert-Butoxycarbonyl);

Boc₂O (Di-tert-butyl dicarbonate);
CSF (Cesium fluoride);

### DCE (1,2-Dichloroethane);

### DCM (Dichloromethane);

DDQ (2,3-Dichloro-5,6-dicyano-1,4-benzoquinone);
DMS (Dimethyl sufide);
ATP (Adenosine triphosphate);
Bis-pinacolatodiboron (4,4,4′,4′,5,5,5′,5′-Octamethyl-2,2′-bi-1,3,2-dioxaborolane);
BSA (Bovine serum albumin);
C18 (Refers to 18-carbon alkyl groups on silicon in HPLC stationary phase)

### CH₃CN (Acetonitrile);

### Cy (Cyclohexyl);

DIPEA (Hünig's base, N-ethyl-N-(1-methylethyl)-2-propanamine);

### Dioxane (1,4-Dioxane);

### DMAP (4Ddimethylaminopyridine);

### DME (1,2-Dimethoxyethane);

### DMF (N,N-Dimethylformamide);

### DMSO (Dimethylsulfoxide);

DPPA (Diphenyl phosphoryl azide);
EtOAc (Ethyl acetate);

### EtOH (Ethanol);

Et₂O (Diethyl ether);
HOAc (Acetic acid);
HPLC (High pressure liquid chromatography);

### HMDS (Hexamethyldisilazide);

IPA (Isopropyl alcohol);
LAH (Lithium aluminum hydride);
LDA (Lithium diisopropylamide);
LHMDS (Lithium hexamethyldisilazide);

### MeOH (Methanol);

MTBE (Methyl tert-butyl ether);
mCPBA (m-Chloroperbezoic acid);
NaHMDS (Sodium hexamethyldisilazide);

### NBS (N-bromosuccinimide);

Pd₂(dba)₃ (Tris(dibenzylideneacetone)dipalladium(0);

### Pd(dppf)Cl₂.DCMComplex ([1,1′-Bis(diphenylphosphino)ferrocene]dichloropalladium(II).

dichloromethane complex);
RPHPLC (Reverse phase high pressure liquid chromatography);
RT (Room temperature);

### Sat. (Saturated)

SGC (Silica gel chromatography);
SM (Starting material);
TCL (Thin layer chromatography);

### TEA (Triethylamine);

TFA (Trifluoroacetic acid); and

### THF (Tetrahydrofuran).

All references to ether are to diethyl ether and brine refers to a saturated aqueous solution of NaCl.

Compound Preparation

The compounds according to Formula (I) are prepared using conventional organic synthetic methods. A suitable synthetic route is depicted below in the following general reaction schemes. All of the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

Compounds of the invention with a fluorine substituted at the 8-position of the isoquinoline were prepared according to Scheme 1. Substituted benzyl amine C is prepared by reacting substituted benzaldehyde A with O-methylhydroxylamine hydrochloride in presence of base to obtain corresponding imine B, which upon reduction afforded the benzyl amine C. Di-substituted amine D was obtained by reductive amination of C and 1,1-dimethoxypropan-2-one C1. Cyclisation of D was performed by reacting with chlorosulfonic acid to obtain isoquinoline E.

Radical bromination of methyl isoquinoline E followed by reacting with sodium periodate gave isoquinoline aldehyde G. In some instances bromination of E resulted in monobromination of the methyl group, and the resulting compound can be converted to to the dibromo compound F by treating further with NBS. Isoquinoline aldehyde G was reacted with variety of alkyl/aryl magnesium bromides to give intermediate H. After conversion to the boronate ester I, palladium catalyzed Suzuki-Miyaura reaction with the bicycloheteroaryl bromide J produced the compound K. Compound K was treated with thionyl chloride followed and the by reduction of halide using zinc acetic acid produced the compound M, which represents the structure of the compounds of the invention. The bicycloheteroaryl bromides J were prepared as per the literature procedures described in J. Med. Chem., 2012, 55 (16), pp 7193-7207 and J. Med. Chem., 2015, 58 (3), pp 1426-1441.

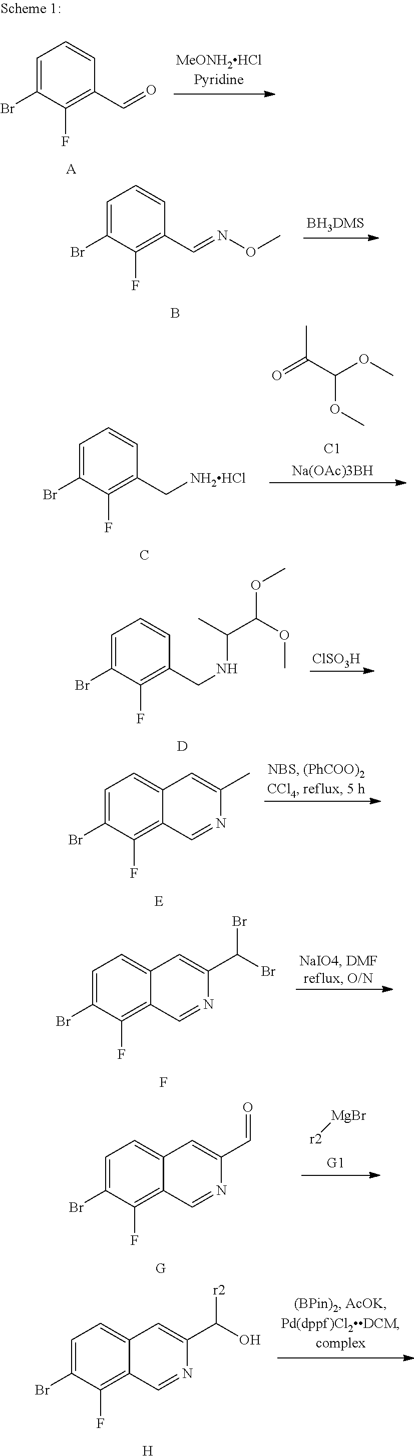

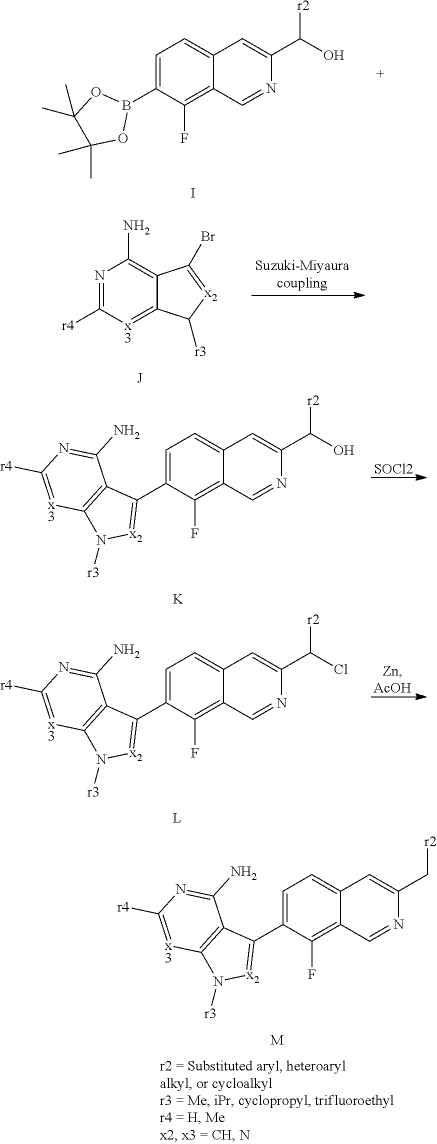

Compounds of the present invention having general formula M can be prepared using an alternate method from intermediate G as described in Scheme 2. Aldehyde intermediate G was reacted with tosyl hydrazine to obtain the corresponding tosyl hydrazone derivative G3. Carbon-Carbon bond formation of common intermediate G3 with variety of boronic acids/boronate esters was performed using bases such as potassium carbonate, or cesium fluoride or potassium phosphate in presence of organic solvent to give intermediate T, following the methods reported by Barluenga et al. (Nat. Chem. 2009, 1, 494-499). Coversion of T to the boronate ester U, followed by palladium catalyzed Suzuki-Miyaura reaction with the bicycloheteroaryl bromides J produced the compounds of the present invention of the general formula M.

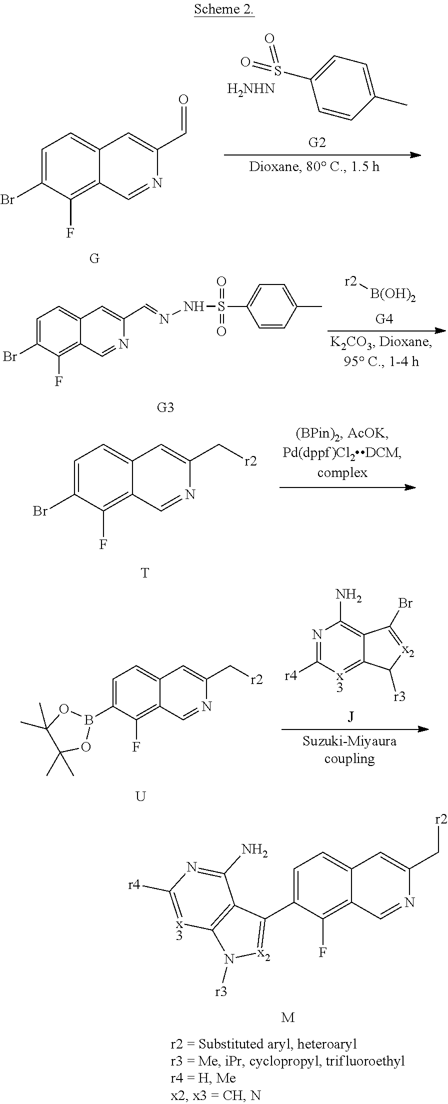

Alternatively, compounds of the invention having the general formula M can be according to scheme 3. 1-bromo-2-fluoro-4-iodobenzene N was converted to corresponding acid O by lithiation followed by quenching with carbon dioxide, which upon treating with thionyl chloride in presence of methanol led to ester P. The ester was reduced to the alcohol and then oxidized using Swern-oxidation conditions to give substituted benzaldehyde R. Benzaldehyde R was converted to t-butyl imine derivative S which was converted to isoquinoline intermediate T by reacting with substituted benzyl acetylene S1 in presence of copper iodide and Palladium(II)bis(triphenylphosphine) dichloride. Boronate ester formation and Suzuki-Miyaura coupling were performed similarly as described in Scheme 2 to obtain compounds M of the present invention.

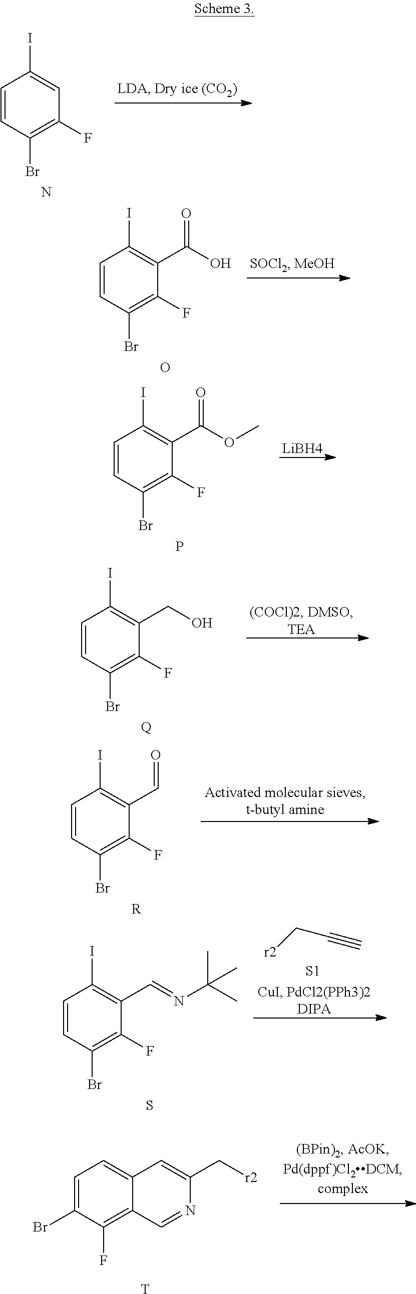

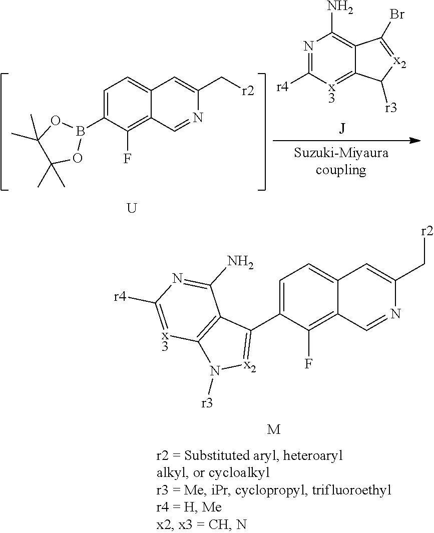

Compounds of the invention without fluorine on to Isoquinoline can be prepared according to scheme 4. Conversion of carboxylic acid V to corresponding ester was performed by using methanol in sulphuric acid. Ring bromination of V1 using NBS gave the corresponding bromo compound V2. Reduction of V2 ester to alcohol using sodium borohydride followed by Swern oxidation gave the corresponding aldehyde V4. Conversion of aldehyde V4 to imine V5 followed by isoquinoline formation and Suzuki-Miyaura coupling as similarly described in Scheme 2 afforded compounds M of the present invention.

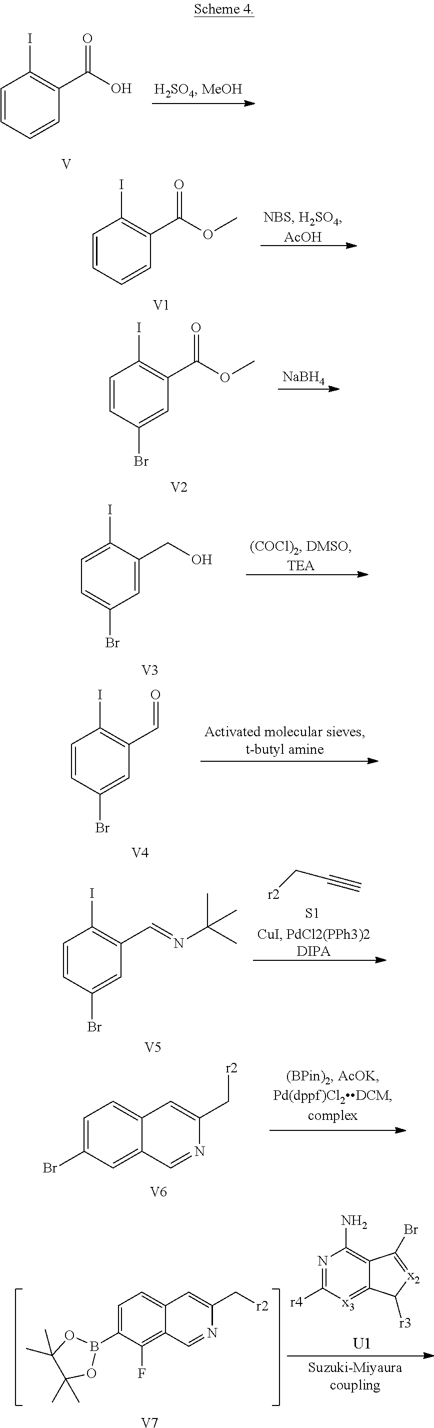

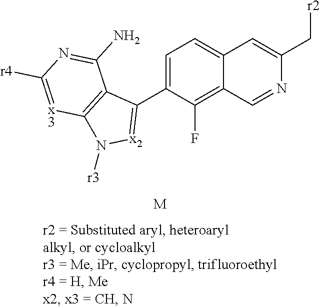

Alternatively, compounds of the invention having the general formula M1 can be prepared by following scheme 5. 4-Bromophthalic acid W1 was reduced to corresponding diol W2, which upon oxidation gave dialdehyde W3. W3 was reacted with diethyl 2-aminomalonate hydrochloride under basic conditions to give the isoquinoline intermediate W4. The reaction to form W4 produces a mixture of regioisomers from which W4 was isolated and used in subsequent reactions. Hydrolysis of ester group on isoquinoline W4 was performed using base such as lithium hydroxide, and the resulting acid W5 was converted to the Weinreb amide W6. Compound W6 was reacted with a variety of Grignard reagents Y to give ketone W7. Reduction of ketone group was performed using hydrazine hydrate to give intermediate V6. Boronate ester formation and Suzuki-Miyaura coupling were performed similarly described in Scheme 2 to obtain compounds of the present invention M1. In a few examples W7 was converted to boronate ester and followed by Suzuki-Miyaura coupling with bicycloheteroaryl bromides J gave compounds of the invention M1.

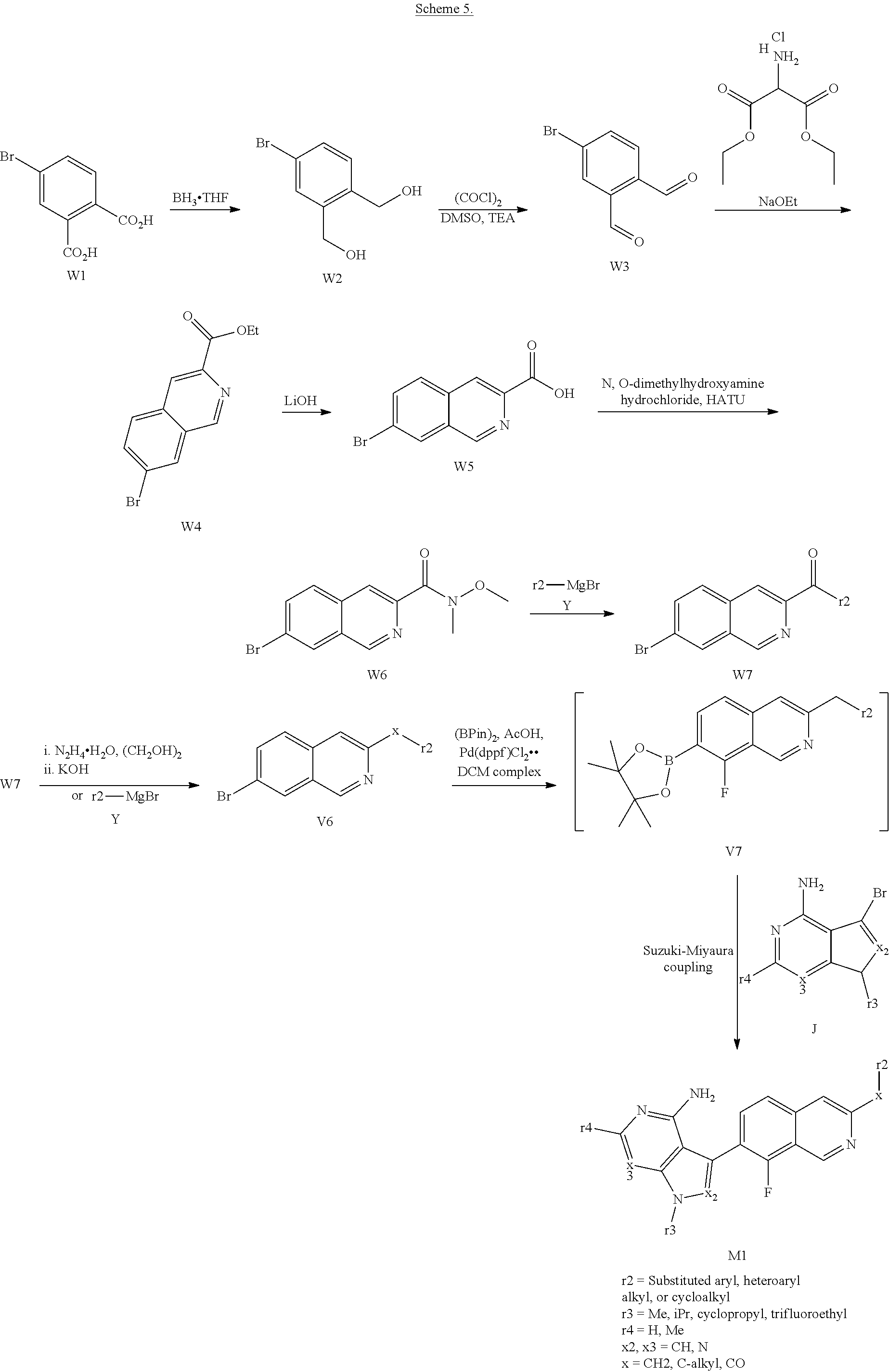

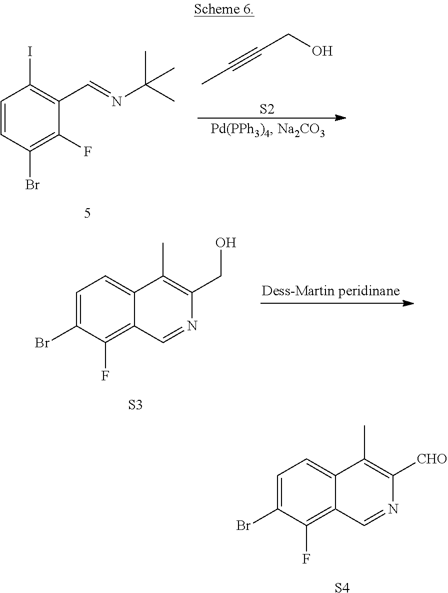

Examples of the present invention with alkyl substitution on the isoquinoline were prepared following scheme 6. Imine derivative S was converted to isoquinoline intermediate S3 by reacting S with but-2-yn-1-ol S2 in presence of Tetrakis(triphenylphosphine)palladium. Isoquinoline alcohol S3 was converted to aldehyde by using an oxidizing agent such Dess-Martin periodinane.

### Methods of Use

The compounds according to Formula (I) and pharmaceutically acceptable salts thereof are inhibitors of PERK. These compounds are potentially useful in the treatment of conditions wherein the underlying pathology is attributable to (but not limited to) activation of the UPR pathway, for example, neurodegenerative disorders, cancer, cardiovascular and metabolic diseases. Accordingly, in another aspect the invention is directed to methods of treating such conditions.

Suitably, the present invention relates to a method for treating or lessening the severity of breast cancer, including inflammatory breast cancer, ductal carcinoma, and lobular carcinoma.

Suitably the present invention relates to a method for treating or lessening the severity of colon cancer.

Suitably the present invention relates to a method for treating or lessening the severity of pancreatic cancer, including insulinomas, adenocarcinoma, ductal adenocarcinoma, adenosquamous carcinoma, acinar cell carcinoma, and glucagonoma.

Suitably the present invention relates to a method for treating or lessening the severity of skin cancer, including melanoma, including metastatic melanoma.

Suitably the present invention relates to a method for treating or lessening the severity of lung cancer including small cell lung cancer, non-small cell lung cancer, squamous cell carcinoma, adenocarcinoma, and large cell carcinoma.

Suitably the present invention relates to a method for treating or lessening the severity of cancers selected from the group consisting of brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

Suitably the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

Suitably the present invention relates to a method for treating or lessening the severity of neurodegenerative diseases/injury, such as Alzheimer's disease, spinal cord injury, traumatic brain injury, ischemic stroke, stroke, Parkinson disease, metabolic syndrome, metabolic disorders, Huntington's disease, Creutzfeldt-Jakob Disease, fatal familial insomnia, Gerstmann-Sträussler-Scheinker syndrome, and related prion diseases, progressive supranuclear palsy, amyotrophic lateral sclerosis, and other diseases associated with UPR activation including: diabetes, myocardial infarction, cardiovascular disease, inflammation, fibrosis, chronic and acute diseases of the liver, fatty liver disease, liver steatosis, liver fibrosis chronic and acute diseases of the lung, lung fibrosis, chronic and acute diseases of the kidney, kidney fibrosis, chronic traumatic encephalopathy (CTE), neurodegeneration, dementia, frontotemporal dementias, tauopathies, Pick's disease, Neimann-Pick's disease, amyloidosis cognitive impairment, atherosclerosis, ocular diseases, and arrhythmias.

Suitably the present invention relates to a method preventing organ damage during and after organ transplantation and in the transportation of organs for transplantation. The method of preventing organ damage during and after organ transplantation will comprise the in vivo administration of a compound of Formula (I). The method of preventing organ damage during the transportation of organs for transplantation will comprise adding a compound of Formula (I) to the solution housing the organ during transportation.

The compounds of this invention inhibit angiogenesis which is implicated in the treatment of ocular diseases. Nature Reviews Drug Discovery 4, 711-712 (September 2005). Suitably the present invention relates to a method for treating or lessening the severity of ocular diseases/angiogenesis. In embodiments of methods according to the invention, the disorder of ocular diseases, including vascular leakage can be: edema or neovascularization for any occlusive or inflammatory retinal vascular disease, such as rubeosis irides, neovascular glaucoma, pterygium, vascularized glaucoma filtering blebs, conjunctival papilloma; choroidal neovascularization, such as neovascular age-related macular degeneration (AMD), myopia, prior uveitis, trauma, or idiopathic; macular edema, such as post surgical macular edema, macular edema secondary to uveitis including retinal and/or choroidal inflammation, macular edema secondary to diabetes, and macular edema secondary to retinovascular occlusive disease (i.e. branch and central retinal vein occlusion); retinal neovascularization due to diabetes, such as retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease; and genetic disorders, such as VonHippel-Lindau syndrome.

In some embodiments, the neovascular age-related macular degeneration is wet age-related macular degeneration. In other embodiments, the neovascular age-related macular degeneration is dry age-related macular degeneration and the patient is characterized as being at increased risk of developing wet age-related macular degeneration.

The methods of treatment of the invention comprise administering an effective amount of a compound according to Formula (I) or a pharmaceutically acceptable salt, thereof to a patient in need thereof.

The invention also provides a compound according to Formula (I) or a pharmaceutically-acceptable salt thereof for use in medical therapy, and particularly in therapy for: cancer, pre-cancerous syndromes, Alzheimer's disease, spinal cord injury, traumatic brain injury, ischemic stroke, stroke, diabetes, Parkinson disease, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation. Thus, in further aspect, the invention is directed to the use of a compound according to Formula (I) or a pharmaceutically acceptable salt thereof in the preparation of a medicament for the treatment of a disorder characterized by activation of the UPR, such as cancer.

By the term “treating” and derivatives thereof as used herein, is meant prophylactic and therapeutic therapy. Prophylactic therapy is appropriate when a subject has, for example, a strong family history of cancer or is otherwise considered at high risk for developing cancer, or when a subject has been exposed to a carcinogen.

As used herein, the term “effective amount” and derivatives thereof means that amount of a drug or pharmaceutical agent that will elicit the biological or medical response of a tissue, system, animal or human that is being sought, for instance, by a researcher or clinician. Furthermore, the term “therapeutically effective amount” and derivatives thereof means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” or “subject” refers to a human or other animal. Suitably the patient or subject is a human.

The compounds of Formula (I) or pharmaceutically acceptable salts thereof may be administered by any suitable route of administration, including systemic administration. Systemic administration includes oral administration, and parenteral administration, Parenteral administration refers to routes of administration other than enteral, transdermal, or by inhalation, and is typically by injection or infusion. Parenteral administration includes intravenous, intramuscular, and subcutaneous injection or infusion.

The compounds of Formula (I) or pharmaceutically acceptable salts thereof may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or overtime as individual patient needs change.

Additionally, the compounds of Formula (I) or pharmaceutically-acceptable salts thereof may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome or overcome a side effect or other difficulty encountered with the compound. Where a —COOH or —OH group is present, pharmaceutically acceptable esters can be employed, for example methyl, ethyl, and the like for —COOH, and acetate maleate and the like for —OH, and those esters known in the art for modifying solubility or hydrolysis characteristics.

The compounds of Formula (I) and pharmaceutically acceptable salts thereof may be co-administered with at least one other active agent known to be useful in the treatment of cancer or pre-cancerous syndromes.

By the term “co-administration” as used herein is meant either simultaneous administration or any manner of separate sequential administration of a PERK inhibiting compound, as described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; cell cycle signaling inhibitors; proteasome inhibitors; and inhibitors of cancer metabolism.

Examples of a further active ingredient or ingredients (anti-neoplastic agent) for use in combination or co-administered with the presently invented PERK inhibiting compounds are chemotherapeutic agents.

Suitably, the pharmaceutically active compounds of the invention are used in combination with a VEGFR inhibitor, suitably 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt thereof, which is disclosed and claimed in in International Application No. PCT/US01/49367, having an International filing date of Dec. 19, 2001, International Publication Number WO02/059110 and an International Publication date of Aug. 1, 2002, the entire disclosure of which is hereby incorporated by reference, and which is the compound of Example 69. 5-[[4-[(2,3-dimethyl-2H-indazol-6-yl)methylamino]-2-pyrimidinyl]amino]-2-methylbenzenesulfonamide can be prepared as described in International Application No. PCT/US01/49367.

Suitably, 5-[[4-[(2,3-dimethyl-2H-indazol-6-yl)methylamino]-2-pyrimidinyl]amino]-2-methylbenzenesulfonamide is in the form of a monohydrochloride salt. This salt form can be prepared by one of skill in the art from the description in International Application No. PCT/US01/49367, having an International filing date of Dec. 19, 2001.

5-[[4-[(2,3-dimethyl-2H-indazol-6-yl)methylamino]-2-pyrimidinyl]amino]-2-methylbenzenesulfonamide is sold commercially as the monohydrochloride salt and is known by the generic name pazopanib and the trade name Votrient®.

Pazopanib is implicated in the treatment of cancer and ocular diseases/angiogenesis. Suitably the present invention relates to the treatment of cancer and ocular diseases/angiogenesis, suitably age-related macular degeneration, which method comprises the administration of a compound of Formula (I) alone or in combination with pazopanib.

In one embodiment, the compound of the invention may be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged.

In one embodiment, the further anti-cancer therapy is surgical and/or radiotherapy.

In one embodiment, the further anti-cancer therapy is at least one additional anti-neoplastic agent.

In a further aspect there is provided a combination comprising a compound of Formula (I) or a pharmaceutically acceptable salt thereof and at least one anti-neoplastic agent.

In a further aspect there is provided a combination comprising a compound of Formula (I) or a pharmaceutically acceptable salt thereof and at least one anti-neoplastic agent, for use in therapy.

In a further aspect there is provided a combination comprising a compound of Formula (I) or pharmaceutically acceptable salt thereof and at least one anti-neoplastic agent, for use in treating cancer and/or pre-cancerous syndromes.

In a further aspect there is provided the use of a combination comprising a compound of Formula (I) or a pharmaceutically acceptable salt thereof and at least one anti-neoplastic agent, in the manufacture of a medicament for the treatment of cancer and/or pre-cancerous syndromes.

In a further aspect there is provided a method of treating cancer, comprising administering to a human in need thereof a therapeutically effective amount of a combination comprising a compound of Formula (I) or a pharmaceutically acceptable salt thereof and at least one anti-neoplastic agent.

In a further aspect there is provided a pharmaceutical composition comprising a combination comprising a compound of Formula (I) or a pharmaceutically acceptable salt thereof and at least one further therapeutic agent, particularly at least one anti-neoplastic agent and one or more of pharmaceutically acceptable carriers, diluents and excipients.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; cell cycle signaling inhibitors; immuno-oncology agents and immunostimulatory agents.

### Anti-Microtubule or Anti-Mitotic Agents:

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

### Platinum Coordination Complexes:

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, oxaliplatin, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma.

### Alkylating Agents:

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease.

Busulfan, 1,4-butanediol dimethanesulfonate, is commercially available as MYLERAN® TABLETS. Busulfan is indicated for the palliative treatment of chronic myelogenous leukemia.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease.

### Antibiotic Anti-Neoplastics:

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas.

### Topoisomerase II Inhibitors:

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children.

### Antimetabolite Neoplastic Agents:

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder.

### Topoisomerase I Inhibitors:

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer.

### Hormones and Hormonal Analogues:

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, estrogens, and anti-estrogens such as fulvestrant, flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

### Signal Transduction Pathway Inhibitors:

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

### Anti-Angiogenic Agents:

(i) Anti-angiogenic agents including non-receptor MEK angiogenesis inhibitors may also be useful. Anti-angiogenic agents such as those which inhibit the effects of vascular edothelial growth factor, (for example the anti-vascular endothelial cell growth factor antibody bevacizumab [Avastin™], and compounds that work by other mechanisms (for example linomide, inhibitors of integrin avβ3 function, endostatin and angiostatin);

### Immunotherapeutic Agents:

Agents used in immunotherapeutic regimens may also be useful in combination with the compounds of Formula (I). Immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies

### Proapoptotic Agents:

Agents used in proapoptotic regimens (e.g., bcl-2 antisense oligonucleotides) may also be used in the combination of the present invention.

### Cell Cycle Signalling Inhibitors

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the combination of the present invention comprises a compound of Formula I or a salt or solvate thereof and at least one anti-neoplastic agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEK angiogenesis inhibitors, immunotherapeutic agents, proapoptotic agents, and cell cycle signaling inhibitors.

In one embodiment, the combination of the present invention comprises a compound of Formula I or a salt or solvate thereof and at least one anti-neoplastic agent which is an anti-microtubule agent selected from diterpenoids and vinca alkaloids.

In a further embodiment, at least one anti-neoplastic agent agent is a diterpenoid.

In a further embodiment, at least one anti-neoplastic agent is a vinca alkaloid.

In one embodiment, the combination of the present invention comprises a compound of Formula I or a salt or solvate thereof and at least one anti-neoplastic agent, which is a platinum coordination complex.

In a further embodiment, at least one anti-neoplastic agent is paclitaxel, carboplatin, or vinorelbine.

In a further embodiment, at least one anti-neoplastic agent is carboplatin.

In a further embodiment, at least one anti-neoplastic agent is vinorelbine.

In a further embodiment, at least one anti-neoplastic agent is paclitaxel.

In one embodiment, the combination of the present invention comprises a compound of Formula I and salts or solvates thereof and at least one anti-neoplastic agent which is a signal transduction pathway inhibitor.

In a further embodiment the signal transduction pathway inhibitor is an inhibitor of a growth factor receptor kinase VEGFR2, TIE2, PDGFR, BTK, erbB2, EGFr, IGFR-1, TrkA, TrkB, TrkC, or c-fms.

In a further embodiment the signal transduction pathway inhibitor is an inhibitor of a serine/threonine kinase rafk, akt, or PKC-zeta.

In a further embodiment the signal transduction pathway inhibitor is an inhibitor of a non-receptor tyrosine kinase selected from the src family of kinases.

In a further embodiment the signal transduction pathway inhibitor is an inhibitor of c-src.

In a further embodiment the signal transduction pathway inhibitor is an inhibitor of Ras oncogene selected from inhibitors of farnesyl transferase and geranylgeranyl transferase.

In a further embodiment the signal transduction pathway inhibitor is an inhibitor of a serine/threonine kinase selected from the group consisting of PI3K.

In a further embodiment the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine (structure below):

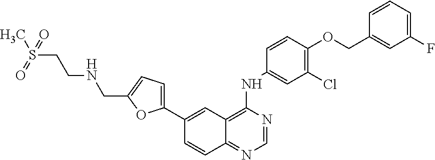 [SMILES: CS(=O)(=O)CCNCc1ccc(-c2ccc3ncnc(Nc4ccc(OCc5cccc(F)c5)c(Cl)c4)c3c2)o1]

In one embodiment, the combination of the present invention comprises a compound of Formula I or a salt or solvate thereof and at least one anti-neoplastic agent which is a cell cycle signaling inhibitor.

In further embodiment, cell cycle signaling inhibitor is an inhibitor of CDK2, CDK4 or CDK6.

### Immunostimulatory Agents:

As used herein “immunostimulatory agent” refers to any agent that can stimulate the immune system. As used herein immunostimulatory agents include, but are not limited to, vaccine adjuvants, such as Toll-like receptor agonists, T-cell checkpoint blockers, such as mAbs to PD-1 and CTL4 and T-cell checkpoint agonist, such as agonist mAbs to OX-40 and ICOS.

Additional examples of a further active ingredient or ingredients (anti-neoplastic agent) for use in combination or co-administered with the presently invented compound of Formula (I) are anti-PD-L1 agents.

Anti-PD-L1 antibodies and methods of making the same are known in the art.

Such antibodies to PD-L1 may be polyclonal or monoclonal, and/or recombinant, and/or humanized.

Exemplary PD-L1 antibodies are disclosed in:
U.S. Pat. No. 8,217,149; Ser. No. 12/633,339;
U.S. Pat. No. 8,383,796; Ser. No. 13/091,936;
U.S. Pat. No. 8,552,154; Ser. No. 13/120,406;
US patent publication No. 20110280877; 13/068337;
US Patent Publication No. 20130309250; 13/892671;
WO2013019906;
WO2013079174;
U.S. application Ser. No. 13/511,538 (filed Aug. 7, 2012), which is the US National Phase of International Application No. PCT/US10/58007 (filed 2010);
and
U.S. application Ser. No. 13/478,511 (filed May 23, 2012).

Additional exemplary antibodies to PD-L1 (also referred to as CD274 or B7-H1) and methods for use are disclosed in U.S. Pat. No. 7,943,743; US20130034559, WO2014055897, U.S. Pat. Nos. 8,168,179; and 7,595,048. PD-L1 antibodies are in development as immuno-modulatory agents for the treatment of cancer.

In one embodiment, the antibody to PD-L1 is an antibody disclosed in U.S. Pat. No. 8,217,149. In another embodiment, the anti-PD-L1 antibody comprises the CDRs of an antibody disclosed in U.S. Pat. No. 8,217,149.

In another embodiment, the antibody to PD-L1 is an antibody disclosed in U.S. application Ser. No. 13/511,538. In another embodiment, the anti-PD-L1 antibody comprises the CDRs of an antibody disclosed in U.S. application Ser. No. 13/511,538.

In another embodiment, the antibody to PD-L1 is an antibody disclosed in application Ser. No. 13/478,511. In another embodiment, the anti-PD-L1 antibody comprises the CDRs of an antibody disclosed in U.S. application Ser. No. 13/478,511.

In one embodiment, the anti-PD-L1 antibody is BMS-936559 (MDX-1105). In another embodiment, the anti-PD-L1 antibody is MPDL3280A (RG7446). In another embodiment, the anti-PD-L1 antibody is MEDI4736.

Additional examples of a further active ingredient or ingredients (anti-neoplastic agent) for use in combination or co-administered with the presently invented compound of Formula (I) are PD-1 antagonist.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any embodiments of the aspects or embodiments of the present invention in which a human individual is to be treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the various aspects and embodiments of the present invention, are described in U.S. Pat. Nos. 7,488,802, 7,521,051, 8,008,449, 8,354,509, 8,168,757, WO2004/004771, WO2004/072286, WO2004/056875, and US2011/0271358.

Specific anti-human PD-1 mAbs useful as the PD-1 antagonist in any of the aspects and embodiments of the present invention include: MK-3475, a humanized IgG4 mAb with the structure described in WHO Drug Information, Vol. 27, No. 2, pages 161-162 (2013) and which comprises the heavy and light chain amino acid sequences shown in FIG. 6; nivolumab, a human IgG4 mAb with the structure described in WHO Drug Information, Vol. 27, No. 1, pages 68-69 (2013) and which comprises the heavy and light chain amino acid sequences shown in FIG. 7; the humanized antibodies h409A11, h409A16 and h409A17, which are described in WO2008/156712, and AMP-514, which is being developed by Medimmune.

Other PD-1 antagonists useful in the any of the aspects and embodiments of the present invention include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

Other examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MED14736, MSB0010718C.

KEYTRUDA/pembrolizumab is an anti-PD-1 antibody marketed for the treatment of lung cancer by Merck. The amino acid sequence of pembrolizumab and methods of using are disclosed in U.S. Pat. No. 8,168,757.

Opdivo/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

Additional examples of a further active ingredient or ingredients (anti-neoplastic agent) for use in combination or co-administered with the presently invented compound of Formula (I) are immuno-modulators.

As used herein “immuno-modulators” refer to any substance including monoclonal antibodies that affects the immune system. The ICOS binding proteins of the present invention can be considered immune-modulators. Immuno-modulators can be used as anti-neoplastic agents for the treatment of cancer. For example, immune-modulators include, but are not limited to, anti-CTLA-4 antibodies such as ipilimumab (YERVOY) and anti-PD-1 antibodies (Opdivo/nivolumab and Keytruda/pembrolizumab). Other immuno-modulators include, but are not limited to, OX-40 antibodies, PD-L1 antibodies, LAG3 antibodies, TIM-3 antibodies, 41BB antibodies and GITR antibodies.

Yervoy (ipilimumab) is a fully human CTLA-4 antibody marketed by Bristol Myers Squibb. The protein structure of ipilimumab and methods are using are described in U.S. Pat. Nos. 6,984,720 and 7,605,238.

CD134, also known as OX40, is a member of the TNFR-superfamily of receptors which is not constitutively expressed on resting naive T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX-40 antibodies, OX-40 fusion proteins and methods of using them are disclosed in US Patent Nos: U.S. Pat. Nos. 7,504,101; 7,758,852; 7,858,765; 7,550,140; 7,960,515; WO2012027328; WO2013028231.

The term “Toll-like receptor” (or “TLR”) as used herein refers to a member of the Toll-like receptor family of proteins or a fragment thereof that senses a microbial product and/or initiates an adaptive immune response. In one embodiment, a TLR activates a dendritic cell (DC). Toll-like receptors (TLRs) are a family of pattern recognition receptors that were initially identified as sensors of the innate immune system that recognize microbial pathogens. TLRs recognize distinct structures in microbes, often referred to as “PAMPs” (pathogen associated molecular patterns). Ligand binding to TLRs invokes a cascade of intra-cellular signaling pathways that induce the production of factors involved in inflammation and immunity. In humans, ten TLR have been identified. TLRs that are expressed on the surface of cells include TLR-I, -2, -4, -5, and -6, while TLR-3, -7/8, and -9 are expressed with the ER compartment. Human DC subsets can be identified on the basis of distinct TLR expression patterns. By way of example, the myeloid or “conventional” subset of DC (mDC) expresses TLRs 1-8 when stimulated, and a cascade of activation markers (e.g. CD80, CD86, MHC class I and II, CCR7), pro-inflammatory cytokines, and chemokines are produced. A result of this stimulation and resulting expression is antigen-specific CD4+ and CD8+ T cell priming. These DCs acquire an enhanced capacity to take up antigens and present them in an appropriate form to T cells. In contrast, the plasmacytoid subset of DC (pDC) expresses only TLR7 and TLR9 upon activation, with a resulting activation of NK cells as well as T-cells. As dying tumor cells may adversely affect DC function, it has been suggested that activating DC with TLR agonists may be beneficial for priming anti-tumor immunity in an immunotherapy approach to the treatment of cancer. It has also been suggested that successful treatment of breast cancer using radiation and chemotherapy requires TLR4 activation.

TLR agonists known in the art and finding use in the present invention include, but are not limited to, the following: Pam3Cys, a TLRI/2 agonist; CFA, a TLR2 agonist; MALP2, a TLR2 agonist; Pam2Cys, a TLR2 agonist; FSL-I, a TLR-2 agonist; Hib-OMPC, a TLR-2 agonist; polyribosinic:polyribocytidic acid (Poly I:C), a TLR3 agonist; polyadenosine-polyuridylic acid (poly AU), a TLR3 agonist; Polyinosinic-Polycytidylic acid stabilized with poly-L-lysine and carboxymethylcellulose (Hiltonol), a TLR3 agonist; bacterial flagellin a TLR5 agonist; imiquimod, a TLR7 agonist; resiquimod, a TLR7/8 agonist; loxoribine, a TLR7/8 agonist; and unmethylated CpG dinucleotide (CpG-ODN), a TLR9 agonist.

Additional TLR agonists known in the art and finding use in the present invention further include, but are not limited to aminoalkyl glucosaminide phosphates (AGPs) which bind to the TLR4 receptor are known to be useful as vaccine adjuvants and immunostimulatory agents for stimulating cytokine production, activating macrophages, promoting innate immune response, and augmenting antibody production in immunized animals. An example of a naturally occurring TLR4 agonist is bacterial LPS. An example of a semisynthetic TLR4 agonist is monophosphoryl lipid A (MPL). AGPs and their immunomodulating effects via TLR4 are disclosed in patent publications such as WO 2006/016997, WO 2001/090129, and/or U.S. Pat. No. 6,113,918 and have been reported in the literature. Additional AGP derivatives are disclosed in U.S. Pat. Nos. 7,129,219, 6,525,028 and 6,911,434. Certain AGPs act as agonists of TLR4, while others are recognized as TLR4 antagonist.

In addition to the immunostimulatory agents described above, the compositions of the present invention may further comprise one or more additional substances which, because of their adjuvant nature, can act to stimulate the immune system to respond to the cancer antigens present on the inactivated tumor cell(s). Such adjuvants include, but are not limited to, lipids, liposomes, inactivated bacteria which induce innate immunity (e.g., inactivated or attenuated I/ster/a monocytogenes), compositions which mediate innate immune activation via, (NOD)-like receptors (NLRs), Retinoic acid inducible gene-based (RIG)-I-like receptors (RLRs), and/or C-type lectin receptors (CLRs). Examples of PAMPs include lipoproteins, lipopolypeptides, peptidoglycans, zymosan, lipopolysaccharide, neisserial porins, flagellin, profillin, galactoceramide, muramyl dipeptide. Peptidoglycans, lipoproteins, and lipoteichoic acids are cell wall components of Gram-positive. Lipopolysaccharides are expressed by most bacteria, with MPL being one example. Flagellin refers to the structural component of bacterial flagella that is secreted by pathogenic and commensal bacterial. rt.-Galactosylceramide (rt.-GalCer) is an activator of natural killer T (NKT) cells. Muramyl dipeptide is a bioactive peptidoglycan motif common to all bacteria.

Because of their adjuvant qualities, TLR agonists are preferably used in combinations with other vaccines, adjuvants and/or immune modulators, and may be combined in various combinations. Thus, in certain embodiments, the herein described compounds of Formula (I) that bind to STING and induce STING-dependent TBKI activation and an inactivated tumor cell which expresses and secretes one or more cytokines which stimulate DC induction, recruitment and/or maturation, as described herein can be administered together with one or more TLR agonists for therapeutic purposes.

Additional examples of a further active ingredient or ingredients (anti-neoplastic agent) for use in combination or co-administered with the presently invented compound of Formula (I) are antibodies to ICOS.

CDRs for murine antibodies to human ICOS having agonist activity are shown in PCT/EP2012/055735 (WO 2012/131004). Antibodies to ICOS are also disclosed in WO 2008/137915, WO 2010/056804, EP 1374902, EP1374901, and EP1125585.

Indoleamine 2,3-dioxygenase 1 (IDO1) is a key immunosuppressive enzyme that modulates the anti-tumor immune response by promoting regulatory T cell generation and blocking effector T cell activation, thereby facilitating tumor growth by allowing cancer cells to avoid immune surveillance. (Lemos H, et al., Cancer Res. 2016 April 15; 76(8):2076-81), (Munn D H, et at., Trends Immunol. 2016 March; 37(3):193-207). Further active ingredients (anti-neoplastic agents) for use in combination or co-administered with the presently invented compounds of Formula (I) are IDO1 inhibitors. Epacadostat, ((Z)—N-(3-bromo-4-fluorophenyl)-N′-hydroxy-4-[2-(sulfamoylamino)ethylamino]-1,2,5-oxadiazole-3-carboxamidine) is a highly potent and selective oral inhibitor of the IDO1 enzyme that reverses tumor-associated immune suppression and restores effective anti-tumor immune responses. Epacadostat is disclosed in U.S. Pat. No. 8,034,953.

Additional examples of a further active ingredient or ingredients (anti-neoplastic agent) for use in combination or co-administered with the presently invented compound of Formula (I) are CD73 inhibitors and A2a and A2b adenosine antagonists.

In one embodiment, the cancer treatment method of the claimed invention includes the co-administration a compound of Formula (I) and/or a pharmaceutically acceptable salt thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, cell cycle signaling inhibitors; proteasome inhibitors; and inhibitors of cancer metabolism.

In one embodiment, a compound of Formula (I) is used as a chemosensitizerto enhance tumor cell killing.

In one embodiment, a compound of Formula (I) is used in combination as a chemosensitizer to enhance tumor cell killing.

In one embodiment, a compound of Formula (I) is used in combination with a modulator of ATF-4.

In one embodiment, a compound of Formula (I) is used in combination with a modulator of ATF-4 to treat diseases/injuries associated with activated unfolded protein response pathways.

In one embodiment, a compound of Formula (I) is used in combination with a modulator of ATF-4 to treat neurodegenerative diseases.

In one embodiment, a compound of Formula (I) is used in combination with a modulator of ATF-4 to treat cancer.

In one embodiment, a compound of Formula (I) is used in combination with a modulator of ATF-4 where the modulator of ATF-4 is ISRIB or another compound that binds to elF2B and enhances global translation.

ISRIB is described in International Application PCT/US2014/029568 having an International Filing Date of Mar. 14, 2014, the International Publication Number WO 2014/144952 and an International Publication Date of Sep. 18, 2014.

One embodiment of this invention provides a combination comprising:
a) a compound of Formula (I) or a pharmaceutically acceptable salt thereof; and
b) an ATF-4 modulating compound.

ATF-4 modulation compounds can be identified by the assays described in International Publication Number WO 2014/144952.

Suitably, the compounds of Formula (I) and pharmaceutically acceptable salts thereof may be co-administered with at least one other active agent known to be useful in the treatment of neurodegenerative diseases/injury.

Suitably, the compounds of Formula (I) and pharmaceutically acceptable salts thereof may be co-administered with at least one other active agent known to be useful in the treatment of diabetes.

Suitably, the compounds of Formula (I) and pharmaceutically acceptable salts thereof may be co-administered with at least one other active agent known to be useful in the treatment of cardiovascular disease.

Suitably, the compounds of Formula (I) and pharmaceutically acceptable salts thereof may be co-administered with at least one other active agent known to be useful in the treatment of ocular diseases.

Suitably, the compounds of Formula (I) and pharmaceutically acceptable salts thereof may be co-administered with at least one other active agent known to be useful for preventing organ damage during and after organ transplantation and in the transportation of organs for transplantation.

### Compositions

The pharmaceutically active compounds within the scope of this invention are useful as PERK inhibitors in mammals, particularly humans, in need thereof.

The present invention therefore provides a method of treating cancer, neurodegeneration and other conditions requiring PERK inhibition, which comprises administering an effective amount of a compound of Formula (I) or a pharmaceutically acceptable salt thereof. The compounds of Formula (I) also provide for a method of treating the above indicated disease states because of their demonstrated ability to act as PERK inhibitors. The drug may be administered to a patient in need thereof by any conventional route of administration, including, but not limited to, intravenous, intramuscular, oral, topical, subcutaneous, transarterial, intradermal, intraocular and parenteral. Suitably, a PERK inhibitor may be delivered directly to the brain by intrathecal or intraventricular route, or implanted at an appropriate anatomical location within a device or pump that continuously releases the PERK inhibitor drug.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical compositions are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-500 mg/kg of active compound, preferably 0.001-100 mg/kg. When treating a human patient in need of a PERK inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, with lower dosages is preferred. Parenteral administration, at high dosages, however, also can be used when safe and convenient for the patient.

Optimal dosages to be administered may be readily determined by those skilled in the art, and will vary with the particular PERK inhibitor in use, the strength of the preparation, the mode of administration, and the advancement of the disease condition. Additional factors depending on the particular patient being treated will result in a need to adjust dosages, including patient age, weight, diet, and time of administration.

When administered to prevent organ damage in the transportation of organs for transplantation, a compound of Formula (I) is added to the solution housing the organ during transportation, suitably in a buffered solution.

The method of this invention of inducing PERK inhibitory activity in mammals, including humans, comprises administering to a subject in need of such activity an effective PERK inhibiting amount of a pharmaceutically active compound of the present invention.

The invention also provides for the use of a compound of Formula (I) or a pharmaceutically acceptable salt thereof in the manufacture of a medicament for use as a PERK inhibitor.

The invention also provides for the use of a compound of Formula (I) or a pharmaceutically acceptable salt thereof in the manufacture of a medicament for use in therapy.

The invention also provides for the use of a compound of Formula (I) or a pharmaceutically acceptable salt thereof in the manufacture of a medicament for use in treating cancer, pre-cancerous syndromes, Alzheimer's disease, spinal cord injury, traumatic brain injury, ischemic stroke, stroke, Parkinson disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, and arrhythmias.

The invention also provides for the use of a compound of Formula (I) or a pharmaceutically acceptable salt thereof in the manufacture of a medicament for use in preventing organ damage during the transportation of organs for transplantation.

The invention also provides for a pharmaceutical composition for use as a PERK inhibitor which comprises a compound of Formula (I) or a pharmaceutically acceptable salt thereof and a pharmaceutically acceptable carrier.

The invention also provides for a pharmaceutical composition for use in the treatment of cancer which comprises a compound of Formula (I) or a pharmaceutically acceptable salt thereof and a pharmaceutically acceptable carrier.

In addition, the pharmaceutically active compounds of the present invention can be co-administered with further active ingredients, such as other compounds known to treat cancer, or compounds known to have utility when used in combination with a PERK inhibitor.

The invention also provides a pharmaceutical composition comprising from 0.5 to 1,000 mg of a compound of Formula (I) or pharmaceutically acceptable salt thereof and from 0.5 to 1,000 mg of a pharmaceutically acceptable excipient.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### Example 1

### 5-(3-Benzylisoquinolin-7-yl)-7-methyl-7H-pyrrolo[2,3-d]pyrimidin-4-amine

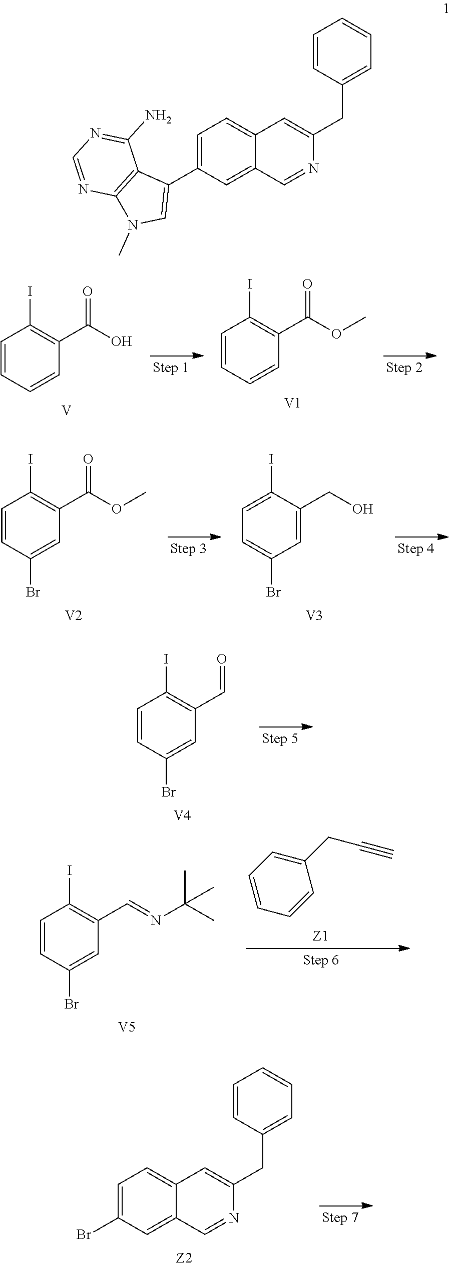

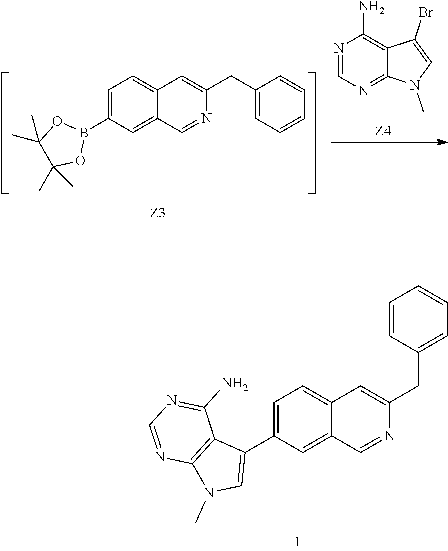

Step 1: To a stirred solution of 2-iodobenzoic acid (10.0 g, 40.32 mmol, 1 equiv) in MeOH (100 mL) was added H₂SO₄ (10 mL) drop wise at 0° C. The reaction mixture was warmed to 90° C. and stirred for 8 hours. The reaction mixture was cooled and concentrated. The residue was basified with saturated sodium bicarbonate at 0° C. and extracted with ethyl acetate (2×150 mL). The organic layer was washed with water and brine solution then dried over sodium sulphate and evaporated to obtain methyl 2-iodobenzoate as colour less liquid (9.0 g, 85%).

¹H NMR (400 MHz, CDCl₃) δ ppm 3.93 (s, 3H), 7.15 (t, J=8.0 Hz, 1H), 7.40 (t, J=7.2 Hz, 1H), 7.80 (d, J=8.0 Hz, 1H), 7.99 (d, J=8.0 Hz, 1H).

Step 2: To a stirred solution of methyl 2-iodobenzoate (5.0 g, 19.08 mmol, 1 equiv) and NBS (3.73 g, 20.99 mmol, 1.1 equiv) in acetic acid (10 mL) was added H₂SO₄ (10 mL) drop wise at 20-40° C. The reaction mixture was stirred for 88 h at room temperature and then heated to 50° C. & stirred for 4 h. The reaction mixture was cooled to 10° C. and quenched with cold water (40 mL) and extracted with DCM (3×50 mL). The organic layer was washed with 5% sodium bicarbonate (2×50 mL), 10% Na₂SO₃ solution (50 mL), and water (50 mL), and then dried over sodium sulphate, and evaporated to obtain methyl 5-bromo-2-iodobenzoate as crude product which was purified over silica gel flash column chromatography. The compound eluted out in 10% ethyl acetate in hexanes. The pure fractions were evaporated to obtain methyl 5-bromo-2-iodobenzoate as off white solid (5 g, 77%). ¹H NMR (400 MHz, CDCl₃) δ ppm 3.94 (s, 3H). 7.26-7.29 (m, 1H), 7.83 (d, J=8.4 Hz, 1H), 7.93 (d, J=8.8 Hz, 1H).

Step 3: To a stirred solution of sodium borohydride (1.1 g, 14.7 mmol, 2 equiv) in ethanol (20 mL) was added methyl 5-bromo-2-iodobenzoate in THF (10 mL) at 5° C. The reaction mixture was warmed to room temperature and stirred for 18 h under nitrogen atmosphere. Additional quantity of sodium borohydride (0.84 g, 22 mmol, 1.5 equiv) was added and the mixture was stirred for 22 h. The reaction mixture was cooled to 0° C., treated with 10 mL of 15% citric acid slowly. The reaction mixture was extracted with DCM (2×75 mL). The organic layer was washed with 15% of aq. NaCl (100 mL), and then dried over sodium sulphate and evaporated to obtain (5-bromo-2-iodophenyl)methanol (4.5 g, 100%) as white solid. ¹H NMR (400 MHz, CDCl₃) δ ppm 1.83-1.88 (m, 1H), 4.63 (s, 2H), 7.12 (dd, J=2.8, 8.4 Hz, 1H), 7.62-7.66 (m, 2H).

Step 4: A solution of oxalyl chloride (1.99 mL, 23.04 mmol, 1.6 equiv) in DCM (25 mL) was cooled to −70° C. and DMSO (2.44 mL, 34.5 mmol, 2.4 equiv) in DCM (25 mL) was added at −65° C. to −70° C. The reaction mixture stirred for 10 minutes under nitrogen atmosphere at −70° C. and then (5-bromo-2-iodophenyl)methanol (4.55 g, 14.4 mmol, 1.0 equiv) in DCM (100 mL) was added. The reaction mixture was stirred at −65° C. for 15 minutes and triethylamine (10 mL, 72 mmol, 5.0 equiv) was added. The reaction mixture was allowed to warm to −10° C. and stir for 1 h. Water (40 mL) was added and the reaction mixture was allowed to warm to room temperature. The organic layer was separated and evaporated to obtain 5-bromo-2-iodobenzaldehyde (4.2 g, 93%) as white solid. ¹H NMR (400 MHz, CDCl₃) δ ppm 7.45 (d, J=7.6 Hz, 1H), 7.81 (d, J=11.6 Hz, 1H), 7.98 (d, J=1.6 Hz, 1H), 9.97 (s, 1H).

Step 5: To a stirred solution of 5-bromo-2-iodobenzaldehyde (4.2 g, 13.5 mmol, 1.0 equiv) in THF (20 mL) was added t-butyl amine (4.26 mL, 40.6 mmol, 3.0 equiv) at room temperature, under nitrogen atmosphere. The reaction mixture was stirred for 40 h at room temperature and evaporated under vacuum to obtain a residue. The residue was dissolved in DCM (100 mL) washed with H2O (50 mL), dried over sodium sulphate and evaporated to obtain (E)-N-(5-bromo-2-iodobenzylidene)-2-methylpropan-2-amine (3.0 g, crude) as a yellow oily compound. ¹H NMR (400 MHz, CDCl₃) δ ppm 1.32 (s, 9H), 7.20 (dd, J=2.8, 8.4 Hz, 1H), 7.68 (d, J=8.4 Hz, 1H), 8.07 (d, J=2.4 Hz, 1H), 8.31 (s, 1H).

Step 6: To a stirred solution of (E)-N-(5-bromo-2-iodobenzylidene)-2-methylpropan-2-amine (1.0 g, 2.73 mmol, 1 equiv) in toluene (20 mL) was added prop-2-yn-1-ylbenzene (0.38 g, 3.26 mmol, 1.2 equiv), followed by copper Iodide (0.1 g, 0.54 mmol, 0.2 equiv), and PdCl₂(PPh₃)₂(0.058 g, 0.08 mmol, 0.03 equiv). The reaction mixture was stirred for 4 h at room temperature under nitrogen atmosphere. Additional copper iodide (0.065 g, 0.35 mmol) was added and the mixture was stirred for 4 hours at 100° C. The reaction mixture was cooled to room temperature, diluted with ethyl acetate (20 mL), filtered over celite and the filtrate was concentrated to obtain the crude product. The crude product was purified over silica gel flash column chromatography. The compound eluted out in 20% EtOAc:Hexanes. The fractions with pure product were evaporated to obtain 3-benzyl-7-bromoisoquinoline (0.5 g, 61%) as brown semi solid. LCMS (ES) m/z=298.0, 300.0 [M+H]⁺. ¹H NMR (400 MHz, CDCl₃) δ ppm 4.38 (s, 2H), 7.25 (s, 1H), 7.32 (s, 4H), 7.40 (s, 1H), 7.60 (d, J=8.8 Hz, 1H), 7.72 (dd, J=1.2, 8.8 Hz, 1H), 8.10 (s, 1H), 9.15 (s, 1H).

Step 7: To a stirred solution of 3-benzyl-7-bromoisoquinoline (0.2 g, 0.67 mmol, 1 equiv) in 1,4-dioxane (10 mL) was added bis(pinacolato)diboron (0.17 g, 067 mmol, 1 equiv), and potassium acetate (0.19 g, 2.01 mmol, 3 equiv). The reaction mixture was degassed with N₂ for 10 minutes. PdCl₂(dppf)-CH₂Cl₂ adduct (0.027 g, 0.033 mmol, 0.05 equiv) was added and the mixture was degassed with N₂ for additional 5 minutes. The reaction mixture was stirred for 3 hour at 100° C. in a sealed vessel. The reaction mixture was cooled to room temperature. 5-bromo-7-methyl-7H-pyrrolo[2,3-d]pyrimidin-4-amine (0.15 g, 0.67 mmol, 1.0 equiv), saturated aqueous NaHCO₃ (4 mL) and PdCl₂(dppf)-CH₂Cl₂ adduct (0.027 g, 0.033 mmol, 0.05 equiv) were added and the reaction mixture was degassed with nitrogen for 5 minutes. The vessel was sealed and the reaction mixture was stirred 12 hours at 100° C. The reaction mixture was cooled to room temperature and filtered through celite. The filtrate was evaporated to obtain crude product which was purified by silica gel flash column chromatography. The compound eluted out as a mixture in 3% methanol in DCM. The fractions were evaporated to obtain 5-(3-benzylisoquinolin-7-yl)-7-methyl-7H-pyrrolo[2,3-d]pyrimidin-4-amine (0.09 g, 36%) as off white solid. LCMS (ES) m/z=366 [M+H]⁺. ¹H NMR (400 MHz, DMSO-d₆) δ ppm 3.76 (s, 3H), 4.26 (s, 2H), 6.14 (br. s., 2H), 7.17-7.24 (m, 1H), 7.26-7.32 (m, 2H), 7.45 (s, 1H), 7.69 (s, 1H), 7.83 (d, J=8.4 Hz, 1H), 7.95 (d, J=8.4 Hz, 1H), 8.06 (s, 1H), 8.17 (s, 1H), 9.25 (s, 1H).

### Example 2

### 5-(3-(3,5-Dimethylbenzyl)isoquinolin-7-yl)-7-methyl-7H-pyrrolo[2,3-d]pyrimidin-4-amine

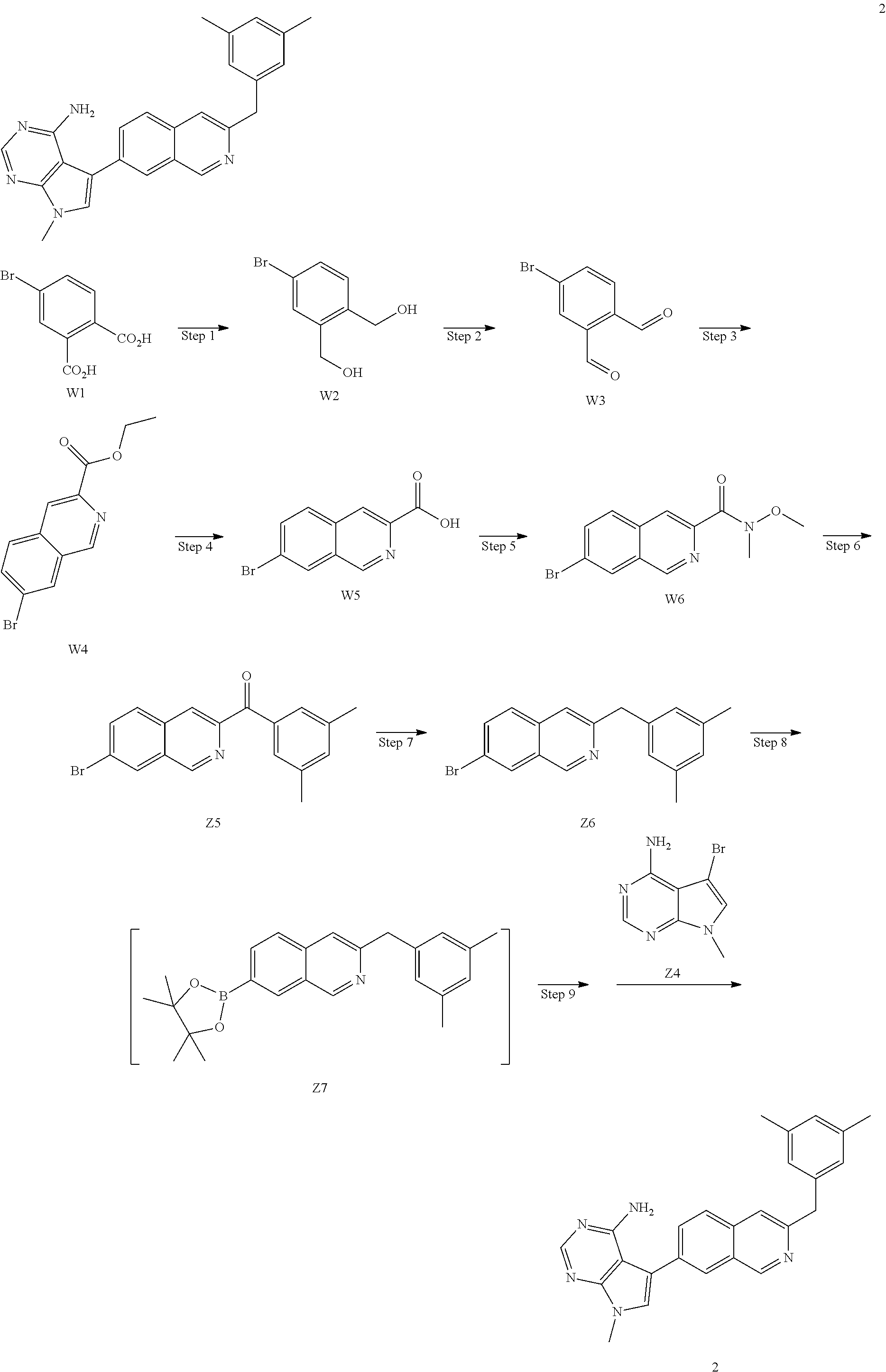

Step 1: To a stirred solution of 4-bromophthalic acid (9.0 g, 37.55 mmol, 1 equiv) in THF (90 mL) was added drop wise BH₃.DMS (35 mL, 375 mmol, 10 equiv) at 0° C. The reaction mixture was warmed to room temperature and stirred for overnight. The reaction mixture was cooled and quenched with MeOH slowly then evaporated to obtain crude product which was purified by silica gel flash column chromatography. The compound eluted out in 1.5% MeOH:DCM. The fractions with product were evaporated to obtain (4-bromo-1,2-phenylene)dimethanol as white solid (6.0 g, 75.9%).

¹H NMR (400 MHz, DMSO-d₆) δ ppm 4.45 (d, J=5.2 Hz, 2H), 4.51 (d, J=5.2 Hz, 2H), 5.12 (t, J=5.6 Hz, 1H), 5.20 (t, J=11.4 Hz, 1H), 7.31 (d, J=8.0 Hz, 1H), 7.40 (t, J=8.0 Hz, 1H), 7.54 (s, 1H).

Step 2: A solution of oxalyl chloride (14.2 mL, 165 mmol, 6.0 equiv) in DCM (120 mL) was cooled to −70° C. and DMSO (11.7 mL, 165 mmol, 6.0 equiv) was added at −65° C. to −70° C. The reaction mixture was stirred for 30 minutes under nitrogen atmosphere at −70° C. (4-bromo-1,2-phenylene)dimethanol (6.0 g, 27.64 mmol, 1.0 equiv) in DCM (25 mL) was added and the reaction mixture stirred at −65° C. for 2 h. Triethylamine (69 mL, 495 mmol, 17.5 equiv) was added and the reaction mixture was allowed to stir at room temperature for 6 h, then treated with water (40 mL). The organic layer was separated and evaporated to obtain crude product, which was purified by silica gel flash column chromatography. The product compound eluted out in 8.0% EtOAc:hexane. The fractions with product were evaporated to obtain (4-bromo-1,2-phenylene)dimethanol (5.0 g, 83.3%) as pale yellow solid.

¹H NMR (400 MHz, DMSO-d₆) δ ppm 7.90 (d, J=8.4 Hz, 1H), 8.07 (d, J=8.4 Hz, 1H), 8.11 (s, 1H), 10.46 (s, 2H).

Step 3: Run 1; To a stirred solution of 4-bromophthalaldehyde (1.6 g, 7.74 mmol, 1.0 equiv) in ethanol (20 mL) was added diethyl 2-aminomalonate hydrochloride (1.63 g, 7.74 mmol, 1.0 equiv) and sodium ethoxide (3.9 mL, 11.61 mmol, 1.5 equiv) at room temperature, and the mixture was stirred for 4 h under nitrogen atmosphere at 80° C. The reaction mixture was cooled to room temperature and quenched with saturated ammonium chloride. The reaction mixture was extracted with ethyl acetate (2×50 mL). The combined organic layers was dried over sodium sulphate and evaporated to obtain crude product.

Run 2; To a stirred solution of 4-bromophthalaldehyde (1.6 g, 7.74 mmol, 1.0 equiv) in ethanol (20 mL) was added diethyl 2-aminomalonate hydrochloride (1.63 g, 7.74 mmol, 1.0 equiv) and sodium ethoxide (3.9 mL, 11.61 mmol, 1.5 equiv) at room temperature, and the mixture was stirred for 4 h under nitrogen atmosphere at 80° C. The reaction mixture was cooled to room temperature, and quenched with saturated ammonium chloride. The reaction mixture was extracted with ethyl acetate (2×50 mL). The combined organic layers was dried over sodium sulphate and evaporated to obtain crude product. The crude products from Run 1 and Run 2 were combined and purified by silica gel flash column chromatography. The compound eluted out in 15-25% EtOAc:Hexanes. The fractions were evaporated to obtain ethyl 7-bromoisoquinoline-3-carboxylate (1.2 g, 28%) as brown solid. ¹H NMR (400 MHz, CDCl₃) δ ppm 1.49 (t, J=7.2 Hz, 3H), 4.51-4.57 (m, 2H), 7.86 (s, 2H), 8.24 (s, 1H), 8.56 (s, 1H), 9.28 (s, 1H).

Step 4: To a stirred solution of ethyl 7-bromoisoquinoline-3-carboxylate (1.2 g, 4.28 mmol, 1.0 equiv) in MeOH:THF:H₂O (2:2:1) (35 mL) was added LiOH monohydrate (0.9 g, 21.42 mmol, 5 equiv) at 0° C. and stirring was continued at room temperature for 0.5 h. The reaction mixture was evaporated and quenched with 1N HCl. The reaction mixture was extracted with 5% MeOH in DCM (3×50 mL), and the combined organics was dried over sodium sulphate, filtered and concentrated to give 7-bromoisoquinoline-3-carboxylic acid (1.0 g, crude) as an off-white solid. LCMS (ES) m/z=252.0, 254.0 [M+H]⁺. ¹H NMR (400 MHz, DMSO-d₆) δ ppm 8.01 (dd, J=2.0, 8.8 Hz, 1H), 8.16 ((d, J=8.8 Hz, 1H), 8.54 (s, 1H), 8.64 (s, 1H), 9.37 (s, 1H), 13.16 (br. s., 1H).

Step 5: To a stirred solution of 7-bromoisoquinoline-3-carboxylic acid (1.0 g, 3.96 mmol, 1.0 equiv) in DMF (20 mL) was added N,O-dimethylhydroxylamine hydrochloride (0.77 g, 7.93 mmol, 2 equiv) and HATU (1.8 g, 4.76 mmol, 1.2 equiv). The reaction mixture was stirred at room temperature for 5 minutes. Triethylamine (1.6 mL, 11.90 mmol, 3 equiv) was added drop-wise and the mixture was then stirred for 40 minutes at room temperature. The reaction mixture was quenched with water (40 mL) and extracted with DCM (3×50 mL). The organic layers were combined, dried over sodium sulphate, filtered and evaporated to obtain crude product, which was purified by silica gel flash column chromatography. The compound eluted out in 1.2% MeOH:DCM. The fractions with product were evaporated to give 7-bromo-N-methoxy-N-methylisoquinoline-3-carboxamide (1.0 g, 90.9%) as white solid. LCMS (ES) m/z=295.0, 297.0 [M+H]⁺. ¹H NMR (400 MHz, DMSO-d6) δ ppm 2.72 (s, 3H), 3.69 (s, 3H), 7.95 (d, J=8.4 Hz, 1H), 8.06 (d, J=8.8 Hz, 1H), 8.15 (s, 1H), 8.50 (s, 1H), 9.32 (s, 1H).

Step 6: To a stirred suspension of magnesium (0.048 g, 2.03 mmol, 1.2 equiv) in THF (20 mL) under nitrogen atmosphere was added 1-bromo-3,5-dimethylbenzene (0.37 g, 2.03 mmol, 1.2 equiv), and a pinch of Iodine was added and the reaction was heated to reflux, stirred for 1 h and cooled to room temperature. In a separate round bottom flask, 7-bromo-N-methoxy-N-methylisoquinoline-3-carboxamide (0.5 g, 1.64 mmol, 1.0 equiv) in THF (20 mL) was cooled to 0° C., the above solution of (3,5-dimethylphenyl)magnesium bromide was added drop wise and the resulting reaction mixture was stirred at room temperature for 1 h. The reaction mixture quenched with water (10 mL) and extracted with ethyl acetate (3×25 mL). The combined organics was dried over sodium sulphate, filtered and concentrated to give (7-bromoisoquinolin-3-yl)(3,5-dimethylphenyl)methanone (0.3 g, 55%) as off white solid. LCMS (ES) m/z=340.0, 342.0 [M+H]⁺. ¹H NMR (400 MHz, CDCl₃) δ ppm 2.38 (s, 6H), 7.62 (s, 1H), 7.87 (s, 2H), 8.25 (s, 1H), 8.40 (s, 1H), 9.26 (s, 1H).

Step 7: Run1; To a stirred solution of (7-bromoisoquinolin-3-yl)(3,5-dimethylphenyl)methanone (0.05 g, 0.146 mmol, 1.0 equiv) in ethylene glycol (3 mL) was added hydrazine hydrate (1.6 g, 31.96 mmol, 219 equiv). The reaction mixture was heated to 150° C. and stirred for 40 minutes. Potassium hydroxide (pulverized) (0.6 g, 10.69 mmol, 73 equiv) was added and the reaction mixture was heated to 180° C.; water was removed using dean-stark condenser. The reaction mixture was stirred for 2 h at 180° C. then cooled to room temperature. Water (30 mL) was added, the reaction mixture was extracted with diethyl ether (2×15 mL). The organic layer was washed with brine (10 mL), dried over sodium sulphate, filtered and concentrated to give 7-bromo-3-(3,5-dimethylbenzyl)isoquinoline (0.045 g, crude) as a brown colored gummy compound. LCMS (ES) m/z=326.0, 328.1[M+H]⁺.

Run 2; To a stirred solution of (7-bromoisoquinolin-3-yl)(3,5-dimethylphenyl)methanone (0.2 g, 0.58 mmol, 1.0 equiv) in ethylene glycol (8 mL) was added hydrazine hydrate (6.35 g, 127 mmol, 219 equiv). The reaction mixture was heated to 150° C. and stirred for 40 minutes. Potassium hydroxide (pulverized) (2.37 g, 42.34 mmol, 73 equiv) was added and the reaction mixture was heated to 180° C.; water was removed using dean-stark condenser. The reaction mixture stirred for 2 h at 180° C. then cooled to room temperature. Water (30 mL) was added and the reaction mixture was extracted with diethyl ether (2×50 mL). The organic layer was washed with brine (25 mL) dried over sodium sulphate, filtered and concentrated to afford the crude product. The crude product from run 1 and run 2 was combined and purified by silica gel flash column chromatography. The compound eluted out in 14% EtOAc:Hexanes. The fractions with product were evaporated to obtain 7-bromo-3-(3,5-dimethylbenzyl)isoquinoline (0.17 g, 71%) as yellow solid. LCMS (ES) m/z=326.0, 328.1[M+H]⁺. ¹H NMR (400 MHz, CDCl₃) δ ppm 2.28 (s, 6H), 4.21 (s, 1H), 6.87 (s, 1H), 6.92 (s, 1H), 7.41 (s, 1H), 7.61 (d, J=8.8 Hz, 1H), 7.70 (d, J=8.8 Hz, 1H) 8.09 (s, 1H), 9.14 (s, 1H).

Step 8: To a stirred solution of 7-bromo-3-(3,5-dimethylbenzyl)isoquinoline (0.16 g, 0.49 mmol, 1 equiv) in 1,4-dioxane (8 mL) was added bis(pinacolato)diboron (0.124 g, 0.49 mmol, 1 equiv), and potassium acetate (0.144 g, 1.47 mmol, 3 equiv). The reaction mixture was degassed with N₂ for 10 minutes. PdCl₂(dppf)-CH₂Cl₂ adduct (0.02 g, 0.024 mmol, 0.05 equiv) was added and the mixture was degassed with N₂ for a further 5 minutes. The reaction mixture was stirred for 5 hours at 100° C. in a sealed vessel. The reaction was cooled to room temperature, 5-bromo-7-methyl-7H-pyrrolo[2,3-d]pyrimidin-4-amine (0.11 g, 0.49 mmol, 1.0 equiv), saturated aqueous NaHCO₃ (3.2 mL) and PdCl₂(dppf)-CH₂Cl₂ adduct (0.02 g, 0.024 mmol, 0.05 equiv) were added and the reaction mixture was degassed with N₂ for 5 minutes. The vessel was sealed and the reaction mixture was stirred for 12 hours at 100° C. The reaction mixture was cooled to room temperature, filtered through celite and the filtrate was evaporated to obtain crude product, which was purified by silica gel flash column chromatography. The compound eluted out in 4% MeOH:DCM. The fractions were evaporated to obtain 5-(3-(3,5-dimethylbenzyl)isoquinolin-7-yl)-7-methyl-7H-pyrrolo[2,3-d]pyrimidin-4-amine (0.04 g, 21%) as white solid. LCMS (ES) m/z=394.2 [M+H]⁺. ¹H NMR (400 MHz, DMSO-d₆) δ ppm 2.21 (s, 6H), 3.76 (s, 3H), 4.13 (s, 2H), 6.15 (br. s., 2H), 6.81 (s, 1H), 6.91 (s, 2H), 7.45 (s, 1H), 7.67 (s, 1H), 7.82 (d, J=8.4 Hz, 1H), 7.95 (d, J=8.4 Hz, 1H), 8.05 (s, 1H), 8.17 (s, 1H), 9.24 (s, 1H).

### Example 3

### 5-(3-Benzyl-8-fluoroisoquinolin-7-yl)-7-methy-7H-pyrrolo[2,3-d]pyrimidin-4-amine

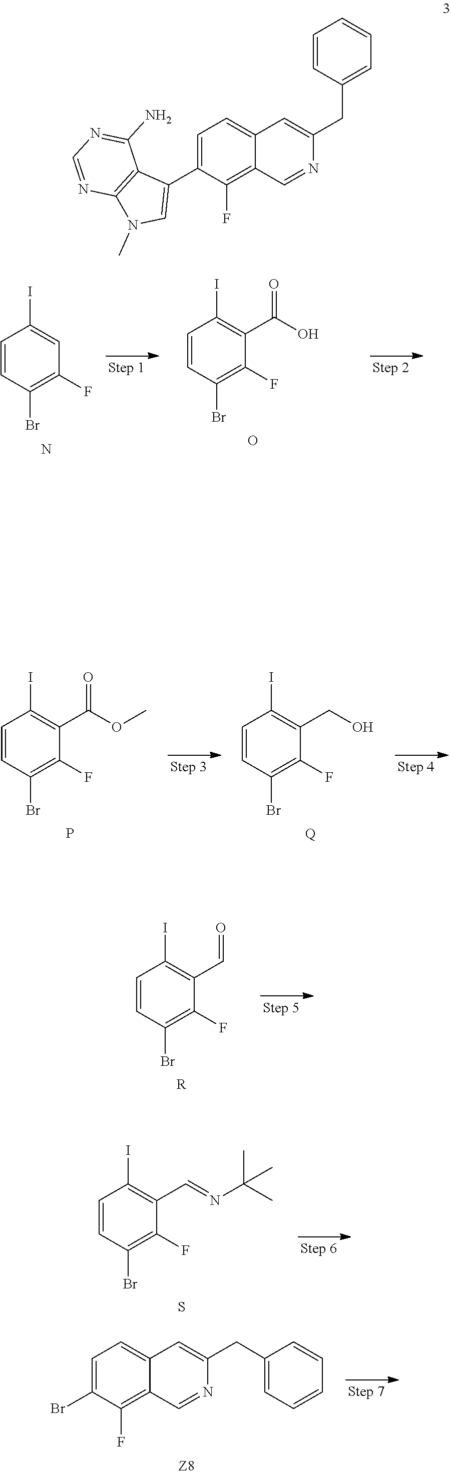

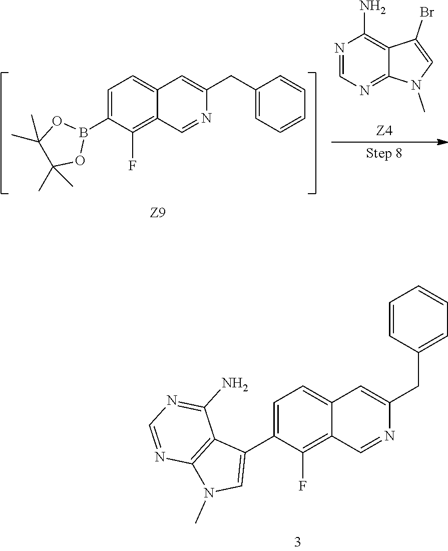

Step 1: To a stirred solution of 1-bromo-2-fluoro-4-iodobenzene (5.0 g, 16.66 mmol, 1 equiv) in THF (50 mL) was added LDA (8.3 mL, 16.66 mmol, 1.0 equiv) drop wise at −78° C. The reaction mixture was stirred for 1 h and then dry ice was added portion wise at −78° C. The reaction mixture was allowed to warm and stir at room temperature overnight. The reaction mixture was quenched with 1N HCl and extracted with 5% MeOH in DCM (3×60 mL). The organic layer was dried over sodium sulphate, filtered and concentrated to give 3-bromo-2-fluoro-6-iodobenzoic acid (3.5 g, 61.4%) as brown solid. LCMS (ES) m/z=344.0, 346.0 [M+H]⁺. ¹H NMR (400 MHz, DMSO-d₆) δ ppm 7.53 (t, J=8.4 Hz, 1H), 7.65 (d, J=8.0 Hz, 1H), 14.18 (s, 1H).

Step 2: To a stirred solution of 3-bromo-2-fluoro-6-iodobenzoic acid (3.3 g, 9.59 mmol, 1 equiv) in DCM (50 mL) was added SOCl₂ (50 mL) drop wise at 0° C. The reaction mixture was warmed to room temperature and stirred for 16 hours. The reaction mixture concentrated and MeOH (50 mL) was added, then the mixture was stirred for 1 h at room temperature. The reaction mixture was evaporated and quenched with saturated sodium bicarbonate at 0° C. and extracted with ethyl acetate (2×150 mL). The combined organic layers was washed with water, brine solution, dried over sodium sulphate and evaporated to obtain methyl 2-iodobenzoate as colour less liquid (3.4 g, 99%). ¹H NMR (400 MHz, CDCl₃) δ ppm 3.91 (s, 3H), 7.60 (t, J=8.0 Hz, 1H), 7.70 (d, J=8.4 Hz, 1H).

Step 3: Run 1; To a stirred solution of methyl 3-bromo-2-fluoro-6-iodobenzoate (0.2 g, 0.55 mmol, 1 equiv) in THF (10 mL) was added LiBH4 (0.55 mL, 1.11 mmol, 2.0 equiv) dropwise at −15° C. The reaction mixture was warmed to room temperature and stirred for 4 h. Water (5 mL) was added, the reaction mixture was extracted with ethyl acetate (2×10 mL). The combined organic layers was dried over sodium sulphate, filtered and concentrated to give crude compound.

Run 2; To a stirred solution of methyl 3-bromo-2-fluoro-6-iodobenzoate (3.0 g, 8.35 mmol, 1 equiv) in THF (30 mL) was added LiBH₄ (8.35 mL, 16.7 mmol, 2.0 equiv) dropwise at −15° C. The reaction mixture was warmed to room temperature and stirred for 4 h. Water (50 mL) was added, the reaction mixture was extracted with ethyl acetate (3×100 mL). The combined organic layers was dried over sodium sulphate, filtered and concentrated to give crude compound. The crude compound from run 1 and run 2 were mixed and purified by silica gel flash column chromatography. The compound eluted out in 5% EtOAc:hexane. The pure fractions were evaporated to obtain (3-bromo-2-fluoro-6-iodophenyl)methanol (1.61 g, 55%) as white solid. ¹H NMR (400 MHz, DMSO-d₆) δ ppm 4.56-4.58 (m, 2H), 5.25 (t, J=5.2 Hz, 1H), 7.40 (t, J=7.6 Hz, 1H), 7.63 (d, J=8.4 Hz, 1H).

Step 4: A stirred solution of oxalyl chloride (0.73 mL, 8.46 mmol, 2.0 equiv) in DCM (15 mL) was cooled to −70° C. and DMSO (0.72 mL, 34.5 mmol, 2.4 equiv) was added at −65° C. to −70° C. The reaction mixture was stirred for 10 minutes under nitrogen atmosphere at −70° C. and then (3-bromo-2-fluoro-6-iodophenyl)methanol (1.4 g, 4.23 mmol, 1.0 equiv) in DCM (10 mL) was added. The reaction mixture was stirred at −65° C. for 15 minutes and triethylamine (2.94 mL, 10.15 mmol, 5.0 equiv) was added. The reaction mixture was allowed to warm to −10° C. and stir for 2 h. Water (10 mL) was added and the reaction allowed to warm to room temperature. The organic layer was separated and evaporated to obtain crude product. The crude compound was purified by silica gel flash column chromatography. The compound eluted out in 5% EtOAc:hexane. The pure fractions were evaporated to obtain 3-bromo-2-fluoro-6-iodobenzaldehyde (1.3 g, 95%) as a white solid. ¹H NMR (400 MHz, DMSO-d₆) δ ppm 7.67-7.71 (m, 1H), 7.82 (d, J=8.4 Hz, 1H), 9.92 (s, 1H).

Step 5: 3-Bromo-2-fluoro-6-iodobenzaldehyde (1.0 g, 3.04 mmol, 1.0 equiv), activated molecular sieves (1.0 g), t-butyl amine (0.95 mL, 9.12 mmol, 3.0 equiv) and toluene (10 mL) were taken in a sealed tube and heated for 24 h at 100° C. The reaction mixture was cooled to room temperature, filtered through celite, washing with ethyl acetate. The filtrate was evaporated to obtain (E)-N-(3-bromo-2-fluoro-6-iodobenzylidene)-2-methylpropan-2-amine (0.9 g, crude) as oily compound.

Step 6: To a stirred solution of (E)-N-(3-bromo-2-fluoro-6-iodobenzylidene)-2-methylpropan-2-amine (0.8 g, 2.08 mmol, 1 equiv) in toluene (10 mL) was added prop-2-yn-1-ylbenzene (0.289 g, 2.49 mmol, 1.2 equiv), copper Iodide (0.04 g, 0.208 mmol, 0.1 equiv), and PdCl₂(PPh₃)₂(0.044 g, 0.06 mmol, 0.03 equiv). The reaction mixture was stirred for 4 h at room temperature under N₂. An additional quantity of copper Iodide (0.04 g, 0.208 mmol, 0.1 equiv) was added and reaction mixture was stirred for 4 hours at 100° C. The reaction mixture was allowed to cool to room temperature, diluted with ethyl acetate (20 mL), and filtered over celite. The filtrate was concentrated to obtain the crude product, which was purified by silica gel flash column chromatography. The compound eluted out as a mixture with an impurity in 20% EtOAc:Hexanes. The fractions containing product were evaporated to obtain 3-benzyl-7-bromo-8-fluoroisoquinoline (0.13 g, crude) as brown colour semi solid. LCMS (ES) m/z=316,318 [M+H]⁺.

Step 7: To a stirred solution of 3-benzyl-7-bromo-8-fluoroisoquinoline (0.13 g, 0.411 mmol, 1 equiv) in 1,4-dioxane (10 mL) was added bis(pinacolato)diboron (0.10 g, 0.411 mmol, 1 equiv), and potassium acetate (0.12 g, 1.23 mmol, 3 equiv). The reaction mixture was degassed with N₂ for 10 minutes. PdCl₂(dppf)-CH₂Cl₂ adduct (0.0167 g, 0.02 mmol, 0.05 equiv) was added and the mixture was degassed with N₂ for 5 minutes. The reaction mixture was stirred for 12 hour at 100° C. in a sealed vessel. The reaction was cooled to room temperature. 5-bromo-7-methyl-7H-pyrrolo[2,3-d]pyrimidin-4-amine (0.094 g, 0.411 mmol, 1.0 equiv), saturated aqueous NaHCO₃ (3 mL) and PdCl₂(dppf)-CH₂Cl₂ adduct (0.0167 g, 0.02 mmol, 0.05 equiv) was added and the reaction mixture was degassed with N₂ for 5 minutes. The vessel was sealed and the reaction mixture was stirred for 8 hour at 100° C. The mixture was filtered through celite and the filtrate was evaporated to obtain crude product, which was purified by silica gel flash column chromatography. The compound eluted out in 3% MeOH:DCM. The fractions containing product were evaporated to obtain 5-(3-benzyl-8-fluoroisoquinolin-7-yl)-7-methyl-7H-pyrrolo[2,3-d]pyrimidin-4-amine (0.012 g, 8%) as an off-white solid. LCMS (ES) m/z=384.2 [M+H]⁺. ¹H NMR (400 MHz, DMSO-d₆) δ ppm 3.76 (s, 3H), 4.26 (s, 2H), 6.13 (br.s., 2H), 7.19 (t, J=6.8 Hz, 1H), 7.27-7.35 (m, 4H), 7.42 (s, 1H), 7.70 (t, J=8.0 Hz, 1H), 7.78-7.80 (m, 2H), 8.15 (s, 1H), 9.41 (s, 1H).

### Example 4

### 5-(3-(3,5-Difluorobenzyl)-8-fluoroisoquinolin-7-yl)-7-methyl-7H-pyrrolo[2,3-d]pyrimidin-4-amine

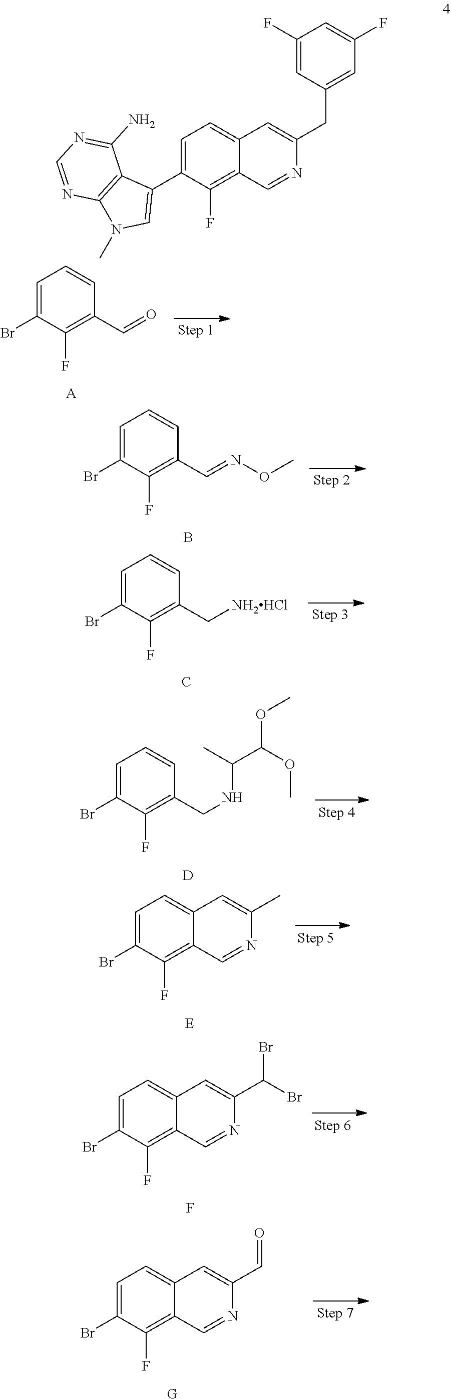

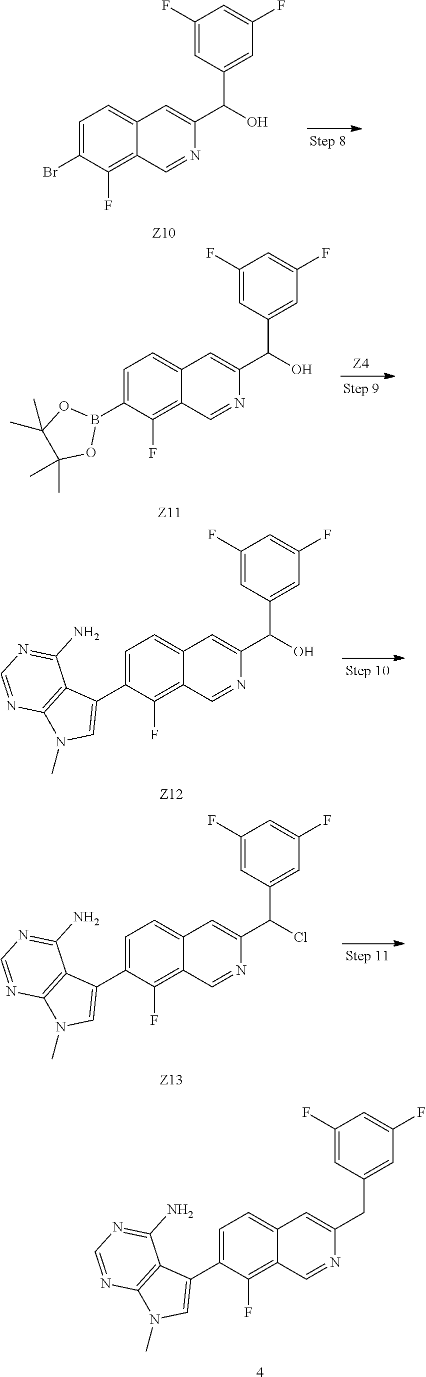

Step 1: Run 1: 3-Bromo-2-fluorobenzaldehyde (5.0 g, 24.63 mmol, 1 equiv) was added to a stirred solution of O-methyl hydroxylamine hydrochloride (2.4 g, 29.55 mmol, 1.2 equiv) and pyridine (7.9 mL, 98.52 mmol, 4 equiv) in DCM (50 mL). The reaction mixture was stirred at room temperature for 1 hour. After consumption of the starting material, the reaction mixture was evaporated under vacuum to obtain crude product. The crude product was purified by flash column chromatography (100-200 Silica gel, 80 g column) using 10% EtOAc in Hexane as mobile phase to afford the desired product (E)-3-bromo-2-fluorobenzaldehyde O-methyl oxime as colorless liquid (5.4 g, 94%). LC-MS (ES) m/z=232.0, 234.0 [M+H]⁺. ¹H NMR (400 MHz, CDCl₃) δ ppm 3.99 (s, 3H), 7.02 (t, J=8 Hz, 1H), 7.52-7.57 (m, 1H), 7.74-7.78 (m, 1H), 8.27 (s, 1H).

Run 2: 3-Bromo-2-fluorobenzaldehyde (9.5 g, 46.79 mmol, 1 equiv) was added to a stirred solution of O-methylhydroxylamine hydrochloride (4.68 g, 56.15 mmol, 1.2 equiv) and pyridine (15 mL, 187.19 mmol, 4 equiv) in DCM (100 mL). The reaction mixture was stirred at room temperature for 1 hour, after consumption of the starting material, the reaction mixture was evaporated in vacuo to obtain crude product. The crude product was purified by flash column chromatography (100-200 Silica gel, 80 g column) using 10% EtOAc in Hexane as mobile phase to afford the desired product (E,Z)-3-bromo-2-fluorobenzaldehyde O-methyl oxime as colorless liquid (10.3 g, 94%). LC-MS (ES) m/z=232.0, 234.0 [M+H]⁺. ¹H NMR (400 MHz, CDCl₃) δ ppm 3.99 (s, 3H), 7.02 (t, J=8 Hz, 1H), 7.52-7.57 (m, 1H), 7.74-7.78 (m, 1H), 8.27 (s, 1H).

Step 2: Run 1: To a stirred solution of (E,Z)-3-bromo-2-fluorobenzaldehyde O-methyl oxime (1.0 g, 4.31 mmol, 1 equiv) in THF (10 mL) was added borane dimethyl sulfide complex (4 mL, 43.10 mmol, 10 equiv) at 0° C., and the mixture was then stirred at 80° C. for 5 h. After consumption of the starting material, the reaction mixture was cooled to 0° C., and quenched with methanol dropwise. 20% HCl in dioxane (5 mL) was added to this reaction mixture, which was then stirred at 90° C. for 1 h. The reaction mixture was evaporated under vacuum to obtain solid product. The solid product was triturated with n-pentane (10 mL) and ether (10 mL) to obtained (3-bromo-2-fluorophenyl)methanamine hydrochloride as off white solid (0.9 g, 87%). LC-MS (ES) m/z=204.0, 206.0 [M+H]⁺. ¹H NMR (400 MHz, DMSO-d₆) δ ppm 4.07 (s, 2H), 7.21 (t, J=8 Hz, 1H), 7.58 (s, 1H), 7.67-7.74 (m, 1H), 8.47 (br.s, 3H).

Run2: To a stirred solution of (E,Z)-3-bromo-2-fluorobenzaldehyde O-methyl oxime (14.3 g, 61.63 mmol, 1 equiv) in THF (150 mL) was added borane dimethyl sulfide complex (58 mL, 616.37 mmol, 10 equiv) at 0° C., and stirred at 80° C. for 5 h. After consumption of the starting material the reaction mixture was cooled to 0° C., quenched with methanol dropwise. 20% HCl in dioxane (50 mL) was added to the reaction mixture, and it was then stirred at 90° C. for 1 h. The reaction mixture was evaporated in vacuo to obtain solid product. The solid product was triturated with n-pentane (50 mL) and ether (50 mL) to obtained (3-bromo-2-fluorophenyl)methanamine hydrochloride as an off white solid (14.2 g, 96%). LC-MS (ES) m/z=204.0, 206.0 [M+H]⁺. ¹H NMR (400 MHz, DMSO-d₆) δ ppm 4.06 (s, 2H), 7.20 (t, J=7.6 Hz, 1H), 7.60 (t, J=7.2 Hz, 1H), 7.71 (t, J=7.2 Hz, 1H), 8.59 (br.s, 3H).

Step 3: To a stirred solution of (3-bromo-2-fluorophenyl)methanamine hydrochloride (15.0 g, 62.5 mmol, 1 equiv) and 1,1-dimethoxypropan-2-one (9.58 g, 81.25 mmol, 1.3 equiv) in DCE (150 mL) was added sodium triacetoxyborohydride (17.22 g, 81.25 mmol, 1.3 equiv) at room temperature and the mixture was stirred overnight. 30% aqueous K₃PO₄ (pH=14) was added to the reaction mixture, the layers were partitioned and the aqueous layer was extracted with EtOAc (2×200 mL), and the organics were combined and washed with brine (100 mL), and dried over Na₂SO₄. The organic solvent was concentrated to give the N-(3-bromo-2-fluorobenzyl)-1,1-dimethoxypropan-2-amine as a colorless liquid (19 g, crude). LC-MS (ES) m/z=306.2, 308.0 [M+H]⁺. ¹H NMR (400 MHz, DMSO-d₆) δ ppm 0.94 (d, J=6.4 Hz, 3H), 1.89 (br.s, 1H), 2.62 (t, J=6 Hz, 1H), 3.22 (s, 3H), 3.25 (s, 3H), 3.73-3.76 (m, 1H), 3.80-3.84 (m, 1H), 4.06 (d, J=5.6 Hz, 1H), 7.09 (t, J=8 Hz, 1H), 7.44 (t, J=6.8 Hz, 1H), 7.53 (t, J=7.2 Hz, 1H).

Step 4: To a stirred solution of chlorosulfuric acid (42 mL, 620.91 mmol, 10 equiv) was added to N-(3-bromo-2-fluorobenzyl)-1,1-dimethoxypropan-2-amine (19 g, 62.09 mmol, 1 equiv) at 0° C. and then the mixture was heated to 100° C. for 10 minutes. The reaction mixture was quenched with ice, basified with 10% NaOH solution and extracted with EtOAc (2×300 mL) and the organics were combined, and then dried over Na₂SO₄. The organic solvent was concentrated to give crude product. The crude product was purified by silica gel flash column chromatography. The compound eluted out in 10% EtOAc:Hexanes. The pure fractions were evaporated to obtain 7-bromo-8-fluoro-3-methylisoquinoline as off white solid (6.3 g, 42%).

LC-MS (ES) m/z=240.0, 242.0 [M+H]⁺. ¹H NMR (400 MHz, CDCl₃) δ ppm 2.70 (s, 3H), 7.40 (d, J=8.8 Hz, 1H), 7.46 (s, 1H), 7.69 (t, J=7.6 Hz, 1H), 9.42 (s, 1H).

Step 5: Run 1: To a stirred solution of 7-bromo-8-fluoro-3-methylisoquinoline (3 g, 12.50 mmol, 1.0 equiv), in CCl₄ (30 mL) was added benzoyl peroxide (0.3 g, 1.25 mmol, 0.1 equiv) and N-bromosuccinimide (4.45 g, 25.00 mmol, 2.0 equiv) at room temperature and the reaction mixture was refluxed for 5 h. After consumption of the starting material the reaction mixture was cooled to room temperature, filtered and the filtrate was concentrated to give crude mixture of 7-bromo-3-(bromomethyl)-8-fluoroisoquinoline and 7-bromo-3-(dibromomethyl)-8-fluoroisoquinoline as brown color liquid (3.6 g, crude). LC-MS (ES) m/z=317.9, 319.9 [M+H]⁺ mono bromo product and LC-MS (ES) m/z=398.0, 399.8 [M+H]⁺ di bromo product.

Run 2: To a stirred solution of 7-bromo-8-fluoro-3-methylisoquinoline (2.9 g, 12.08 mmol, 1.0 equiv), in CCl4 (30 mL) was added benzoyl peroxide (0.29 g, 1.20 mmol, 0.1 equiv) and N-bromosuccinimide (4.3 g, 24.16 mmol, 2.0 equiv) at room temperature and the reaction mixture was refluxed for 5 h. After consumption of the starting material the reaction mixture was cooled to room temperature, filtered and the filtrate was concentrated to give a crude mixture of 7-bromo-3-(bromomethyl)-8-fluoroisoquinoline and 7-bromo-3-(dibromomethyl)-8-fluoroisoquinoline as brown color liquid (3 g, crude). LC-MS (ES) m/z=317.9, 319.9 [M+H]⁺ mono bromo product and LC-MS (ES) m/z=398.0, 399.8 [M+H]⁺ di bromo product.

Step 6: Run 1: To a stirred solution of 7-bromo-3-(bromomethyl)-8-fluoroisoquinoline and 7-bromo-3-(dibromomethyl)-8-fluoroisoquinoline (3.6 g, 9.04 mmol, 1 equiv) in DMF (30 mL) was added NaIO₄ (1.9 g, 9.04 mmol, 1 equiv) at room temperature and the reaction mixture was refluxed at 160° C. for overnight. After consumption of the starting material the reaction mixture was cooled to room temperature, and diluted with ice water (200 mL) and extracted with EtOAc (2×200 mL). The organics were combined and dried over Na₂SO₄. The organic solvent was concentrated to give crude product. The crude product was purified by silica gel flash column chromatography. The compound eluted out in 10% EtOAc:Hexanes. The pure fractions were evaporated to obtain 7-bromo-8-fluoroisoquinoline-3-carbaldehyde (1.2 g, crude). LC-MS (ES) m/z=254.0, 256.0 [M+H]⁺. ¹H NMR (400 MHz, CDCl₃) δ ppm 7.72 (d, J=8.8 Hz, 1H), 7.89-7.92 (m, 1H), 8.36 (s, 1H), 9.64 (s, 1H), 10.28 (s, 1H).

Run2: To a stirred solution of 7-bromo-3-(bromomethyl)-8-fluoroisoquinoline and 7-bromo-3-(dibromomethyl)-8-fluoroisoquinoline (3 g, 9.4 mmol, 1 equiv) in DMF (30 mL) was added NalO₄ (2 g, 9.4 mmol, 1 equiv) at room temperature and the reaction mixture was refluxed at 160° C. for overnight. After consumption of the starting material the reaction mixture was cooled to room temperature, diluted with with ice water (200 mL) and extracted with EtOAc (2×200 mL). The organics were combined and dried over Na₂SO₄. The organic solvent was concentrated to give crude product. The crude product was purified by silica gel flash column chromatography. The compound eluted out in 10% EtOAc:Hexanes. The pure fractions were evaporated to obtain 7-bromo-8-fluoroisoquinoline-3-carbaldehyde (1.25 g, 52%). LC-MS (ES) m/z=254.0, 256.0 [M+H]⁺. ¹H NMR (400 MHz, DMSO-d₆) δ ppm 7.72 (d, J=8.8 Hz, 1H), 7.86-7.92 (m, 1H), 8.36 (s, 1H), 9.64 (s, 1H), 10.28 (s, 1H).

Step 7: To a stirred solution of 7-bromo-8-fluoroisoquinoline-3-carbaldehyde (1.5 g, 5.9 mmol, 1 equiv) in THF (20 mL) was added 0.5 M (3,5-difluorophenyl)magnesium bromide in THF (23 mL, 11.81 mmol, 2 equiv) drop wise at 0° C. The reaction mixture was stirred at room temperature for overnight, and quenched with saturated NH₄Cl (50 mL) at 0° C. The reaction mixture was extracted with EtOAc (2×100 mL), and the organics were combined and washed with brine solution (100 mL). The organic solvent was concentrated to give crude product. The crude product was purified by silica gel flash column chromatography.

The compound eluted out in 10% EtOAc:Hexanes. The pure fractions were evaporated to obtain (7-bromo-8-fluoroisoquinolin-3-yl)(3,5-difluorophenyl)methanol (1.25 g, 57%). LC-MS (ES) m/z=368.0, 370.0 [M+H]⁺. ¹H NMR (400 MHz, DMSO-d₆) δ ppm 5.91 (d, J=4 Hz, 1H), 6.49 (d, J=4 Hz, 1H), 7.04 (t, J=8.8 Hz, 1H), 7.12 (d, J=7.2 Hz, 2H), 7.84 (d, J=8.4 Hz, 1H), 7.96 (t, J=8.4 Hz, 1H), 8.11 (s, 1H), 9.37 (s, 1H).

Step 8: To a stirred solution of (7-bromo-8-fluoroisoquinolin-3-yl)(3,5-difluorophenyl)methanol (1.25 g, 3.39 mmol, 1 equiv) in 1,4-dioxane (40 mL) was added bis(pinacolato)diboron (1.29 g, 5.09 mmol, 1.5 equiv), and potassium acetate (0.83 g, 8.49 mmol, 2.5 equiv). The reaction mixture was degassed with N₂ for 15 min. PdCl₂(dppf)-CH₂Cl₂ adduct (0.138 g, 0.16 mmol, 0.05 equiv) was added. The reaction mixture was stirred for 5 hours at 100° C. in a sealed vessel. The reaction mixture was filtered over celite and the filtrate was concentrated to obtain crude product. The crude product was purified using silica gel flash column chromatography. The compound eluted out in 20-50% EtOAc:Hexanes. The pure fractions were evaporated to obtain (3,5-difluorophenyl)(8-fluoro-7-(4,4,5,5-tetramethyl-1,3,2-dioxaborolan-2-yl)isoquinolin-3-yl)methanol as light brown liquid (1.25 g, crude). LCMS (ES) m/z=334.1 [M+H]⁺-82 Step 9: To a stirred solution of (3,5-difluorophenyl)(8-fluoro-7-(4,4,5,5-tetramethyl-1,3,2-dioxaborolan-2-yl)isoquinolin-3-yl)methanol (1.25 g, 3.01 mmol, 1 equiv), 5-bromo-7-methyl-7H-pyrrolo[2,3-d]pyrimidin-4-amine (0.68 g, 3.01 mmol, 1 equiv) and potassium phosphate (1.27 g, 6.02 mmol, 2 equiv) in 1,4-dioxane:water (30 mL: 10 mL), was added Pd₂(dba)₃ (0.13 g, 0.15 mmol, 0.05 equiv) and the reaction mixture was degassed with N₂ for 5 min. Tri-tert-butylphosphonium tetrafluoroborate (0.08 g, 0.3 mmol, 0.1 equiv) was added and the reaction mixture was further degassed for 5 min. The vial was sealed and the reaction mixture was heated to 100° C. overnight. The reaction mixture was cooled & filtered through celite and the filtrate was concentrated to obtain crude compound. Crude compound was purified by flash column chromatography using a silica gel column, and the compound was eluted at 3% MeOH:DCM, the pure fractions were evaporated to obtain, (7-(4-amino-7-methyl-7H-pyrrolo[2,3-d]pyrimidin-5-yl)-8-fluoroisoquinolin-3-yl)(3,5-difluorophenyl)methanol (0.6 g, crude) as light yellow color liquid. LCMS (ES) m/z=436.1 [M+H]⁺.

Step 10: To a stirred solution of (7-(4-amino-7-methyl-7H-pyrrolo[2,3-d]pyrimidin-5-yl)-8-fluoroisoquinolin-3-yl)(3,5-difluorophenyl)methanol (0.6 g, 1.37 mmol, 1 equiv) in DCM (10 mL) was added thionyl chloride (5 mL) dropwise at 0° C. The reaction mixture was stirred at room temperature for 2 h. The reaction mixture was concentrated, and diluted with DCM (100 mL), washed with saturated NaHCO₃ and brine solution. The organic solvent was concentrated to give crude product. The crude product was purified by silica gel flash column chromatography. The compound eluted out in 2% MeOH:DCM. The pure fractions were evaporated to obtain 5-(3-(chloro(3,5-difluorophenyl)methyl)-8-fluoroisoquinolin-7-yl)-7-methyl-7H-pyrrolo[2,3-d]pyrimidin-4-amine (0.3 g, crude). LC-MS (ES) m/z=454.1[M+H]⁺. ¹H NMR (400 MHz, DMSO-d6) δ ppm 3.76 (s, 3H), 6.18 (br.s, 2H), 6.70 (s, 1H), 7.21 (t, J=9.2 Hz, 1H), 7.35 (d, J=7.2 Hz, 2H), 7.44 (s, 1H), 7.78 (t, J=8 Hz, 1H), 7.91-7.93 (m, 1H), 8.15 (d, J=4.8 Hz, 2H), 9.48 (s, 1H).

Step 11: To a stirred solution of 5-(3-(chloro(3,5-difluorophenyl)methyl)-8-fluoroisoquinolin-7-yl)-7-methyl-7H-pyrrolo[2,3-d]pyrimidin-4-amine (0.3 g, 0.66 mmol, 1 equiv) in NMP (10 mL) and AcOH (5 mL) was added Zinc powder (0.64 g, 9.93 mmol, 15 equiv) at room temperature and the mixture was heated at 110° C. for 2 hours. The reaction mixture was cooled and basified with saturated NaHCO₃ solution. EtOAc (200 mL) was added and the mixture was filtered through a celite bed. The organic layer was separated, dried over Na₂SO₄ and concentrated to obtain crude compound. Crude compound was purified by flash column chromatography using silica gel column, and the compound was eluted at 2% MeOH:DCM, the pure fractions were evaporated to obtain 5-(3-(3,5-difluorobenzyl)-8-fluoroisoquinolin-7-yl)-7-methyl-7H-pyrrolo[2,3-d]pyrimidin-4-amine (0.045 g, 16%) as an off-white. LCMS (ES) m/z=420.2 [M+H]+. ¹H NMR (400 MHz, DMSO-d6) δ ppm 3.75 (s, 3H), 4.29 (s, 2H), 6.14 (br. s, 2H), 7.03-7.05 (m, 3H), 7.41 (s, 1H), 7.71 (t, J=8 Hz, 1H), 7.79-7.83 (m, 2H), 8.14 (s, 1H), 9.41 (s, 1H).

### Example 5

### 7-cyclopropyl-5-(3-(2,3-difluorobenzyl)-8-fluoroisoquinolin-7-yl)-7H-pyrrolo[2,3-d]pyrimidin-4-amine

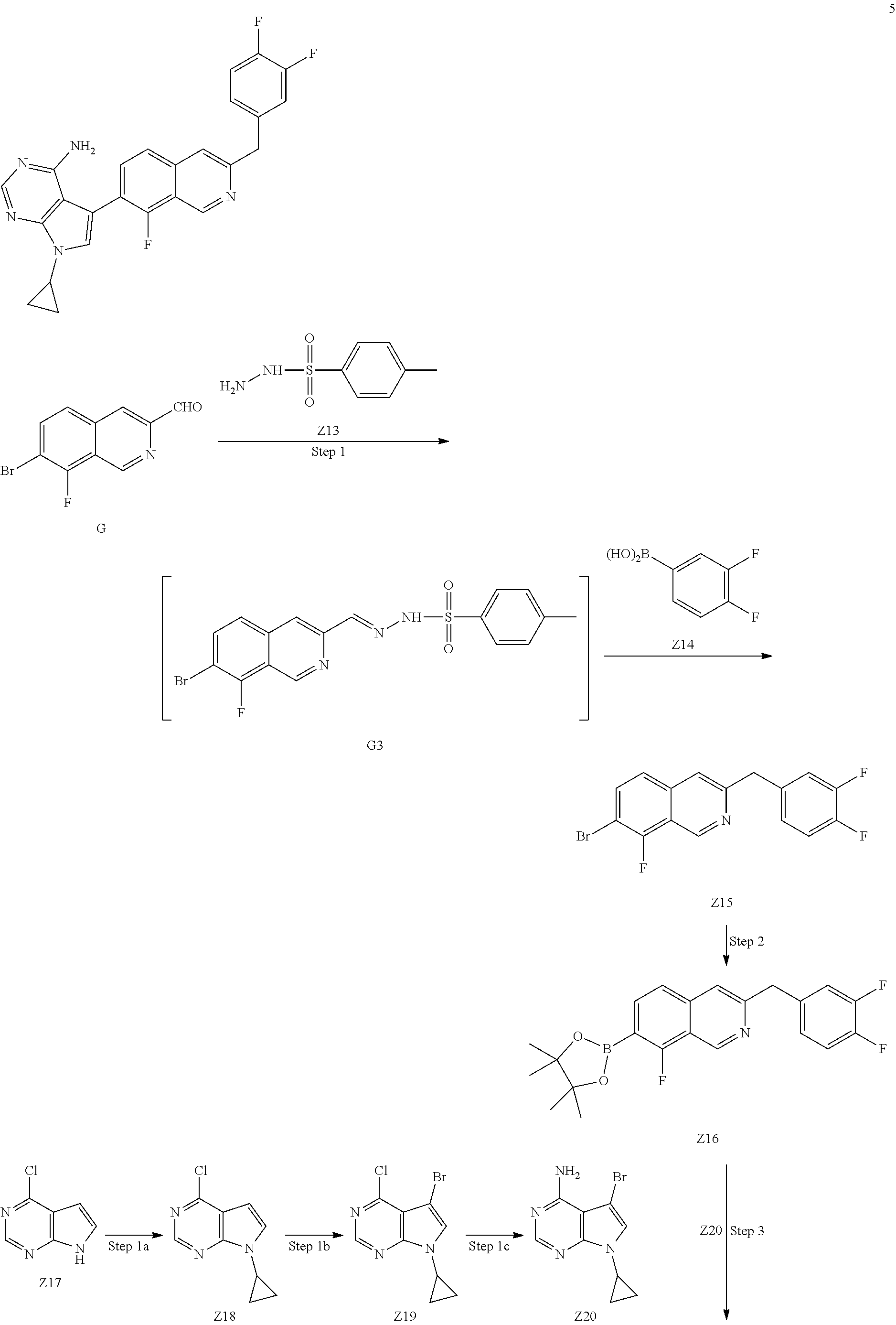

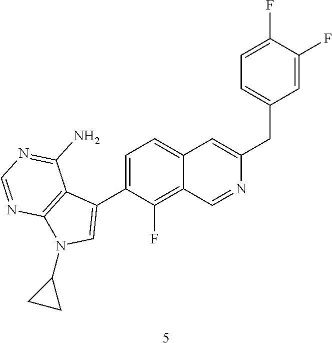 [SMILES: Nc1ncnc2c1c(-c1ccc3cc(Cc4ccc(F)c(F)c4)ncc3c1F)cn2C1CC1 ; "5"]

Step 1a: Run1: To a stirred suspension of 4-chloro-7H-pyrrolo[2,3-d]pyrimidine (0.25 g, 1.63 mmol, 1.0 eq), Cyclopropyl boronic acid (0.28 g, 3.27 mmol, 2.0 eq), and sodium carbonate (0.35 g, 3.27 mmol, 2.0 eq) in DCE (5 mL) at room temperature was added a suspension of Cu(OAc)2 (0.29 g, 1.63 mmol, 1.0 eq) and 2, 2′-Bipyridyl (0.25 g, 1.63 mmol, 1.0 eq) in hot DCE (3 mL). The mixture was heated to 70° C. and stirred for 5 h. The reaction mixture was cooled to room temperature and 1N HCl was added. The organic phase was separated and the aqueous phase was extracted with DCM (3×30 mL). The combined organic layers was washed with brine, dried over Na2SO4, filtered and evaporated.

Run2: To a stirred suspension of 4-chloro-7H-pyrrolo[2,3-d]pyrimidine (2.50 g, 16.27 mmol, 1.0 eq), Cyclopropyl boronic acid (2.80 g, 32.552 mmol, 2.0 eq), and sodium carbonate (3.45 g, 32.55 mmol, 2.0 eq) in DCE (30 mL) at room temperature was added a suspension of Cu(OAc)2 (2.95 g, 16.27 mmol, 1.0 eq) and 2, 2′-Bipyridyl (2.54 g, 16.27 mmol, 1.0 eq) in hot DCE (20 mL). The mixture was heated to 70° C. and stirred for 5 h. The reaction mixture was cooled to room temperature and 1N HCl was added. The organic phase was separated and the aqueous phase was extracted with DCM (3×30 mL). The combined organic layers was washed with brine, dried over Na2SO₄, filtered and evaporated. The crude product was purified by silica gel flash chromatography. The desired product was eluted out in 12% EtOAc in Hexane. Fractions containing pure product were combined and concentrated in vacuo to afford the desired product 4-chloro-7-cyclopropyl-7H-pyrrolo[2,3-d]pyrimidine (1.85 g, 53%) as an off-white solid. LC-MS (ES) m/z=194.1 [M+H]⁺. ¹H NMR (400 MHz, CDCl3) δ 8.67 (s, 1H), 7.23 (s, 1H), 6.54 (s, 1H), 3.58-3.49 (m, 1H), 1.21-1.18 (m, 2H), 1.12-1.05 (s, 2H).

Step 1b: To a stirred solution of 4-chloro-7-cyclopropyl-7H-pyrrolo[2,3-d]pyrimidine (1.85 g, 9.55 mmol, 1.0 eq) in DCM at 0° C. was added NBS (2.04 g, 11.47 mmol, 1.2 eq) slowly.

The mixture was allowed to stir at room temperature for 2 h. After the consumption of starting material, the reaction mixture was diluted with DCM, and washed with water. The organic phase was washed with brine, dried over Na₂SO₄, filtered and evaporated. The crude product was purified by Silica gel flash chromatography. The desired product was eluted out in 12% EtOAc in Hexane. Pure product fractions were combined and concentrated in vacuo to afford the desired product 5-bromo-4-chloro-7-cyclopropyl-7H-pyrrolo[2,3-d]pyrimidine (2.28 g, 88%) as an off-white fluffy solid. LC-MS (ES) m/z=272.0, 274.0 [M+H]⁺. ¹H NMR (400 MHz, CDCl3) δ 8.66 (s, 1H), 72.8 (s, 1H), 3.54-3.48 (m, 1H), 1.28-1.16 (m, 2H), 1.09-1.05 (m, 2H).

Step 1c: To a solution of 5-bromo-4-chloro-7-cyclopropyl-7H-pyrrolo[2,3-d]pyrimidine (2.28 g, 8.37 mmol, 1.0 eq) in Dioxane (10 mL) in a stainless steel Autoclave vessel (Steel bomb) was added 25% aq.NH₃ (40 mL) and the vessel was closed and heated to 100° C. overnight. After 14 h LCMS showed complete conversion. The reaction mixture was cooled to 25° C. and the suspension was filtered. The cake was washed with water (3×5 mL) followed by pentane (10 ml), and dried under vacuum thoroughly to afford the desired product 5-bromo-7-cyclopropyl-7H-pyrrolo[2,3-d]pyrimidin-4-amine (1.58 g, 75%) as a beige solid. LC-MS (ES) m/z=253.0, 255.0 [M+H]⁺. ¹H NMR (400 MHz, CDCl3) δ 8.09 (s, 1H), 7.32 (s, 1H), 6.64 (br. s., 2H), 3.52-3.45 (m, 1H), 0.98-0.92 (m, 4H).

Step 1: A solution of 7-bromo-8-fluoroisoquinoline-3-carbaldehyde (0.5 g, 1.96 mmol, 1.0 eq) and 4-methylbenzenesulfonohydrazide (0.40 g, 2.16 mmol, 1.1 eq) in 1,4-Dioxane (12 mL) was stirred at 80° C. for 1.5 h. Potassium carbonate (0.408 g, 2.95 mmol, 1.5 eq) and (3,4-difluorophenyl)boronic acid (0.47 g, 2.95 mmol, 1.5 eq) were added to the reaction mixture. The system was heated to 95-100° C. and stirred for 1.5 h. The reaction was allowed to room temperature, and the solvent was evaporated. The crude mass was partitioned between DCM and sat. NaHCO₃. The two layers were separated and the aq. phase was extracted with DCM. The combined organic layers was washed with sat. NaHCO₃, brine and then dried over MgSO₄ and filtered. The solvent was removed under reduced pressure and the crude product was purified by silica gel flash chromatography. The desired product was eluted in 6% EtOAc in Hexane. The collected fractions with pure product were combined and concentrated in vacuo to afford the desired product 7-bromo-3-(3,4-difluorobenzyl)-8-fluoroisoquinoline (0.20 g, 29%) as a yellow solid. LC-MS (ES) m/z=352.0, 354.0 [M+H]⁺. ¹H NMR (400 MHz, CDCl3) δ 4.25 (s, 2H), 7.04-6.98 (m, 1H), 7.13-7.06 (m, 2H), 9.43 (s, 1H), 7.43-7.41 (m, 2H), 7.73 (t, J=8.0 Hz, 1H).

Step 2: A mixture of 7-bromo-3-(3,4-difluorobenzyl)-8-fluoroisoquinoline (0.19 g, 0.54 mmol, 1.0 eq), 4,4,4′,4′,5,5,5′,5′-octamethyl-2,2′-bi(1,3,2-dioxaborolane) (0.20 g, 081 mmol, 1.5 eq), potassium acetate (0.13 g, 1.35 mmol, 2.5 eq) and PdCl2(dppf)-CH₂Cl₂ adduct (22 mg, 0.03 mmol, 0.05 equiv) in 12 mL of 1,4-dioxane in a 50 mL single neck round bottom flask, was degassed under Argon for 5 min. and then heated in an oil bath at 100° C. for 12 h. The mixture was cooled to room temperature and filtered through Celite, the Celite pad was washed with DCM. The filtrate was dried over Na₂SO₄, filtered, and concentrated in vacuo. The crude product was purified by silica gel flash chromatography. The desired product was eluted in 9% EtOAc in hexane. Fractions containing pure product were combined and concentrated to afford the desired product 3-(3,4-difluorobenzyl)-8-fluoro-7-(4,4,5,5-tetramethyl-1,3,2-dioxaborolan-2-yl)isoquinoline (92 mg, 43%) as a white solid. LC-MS (ES) m/z=318.1 [M+H]⁺-82. ¹H NMR (400 MHz, CDCl3) δ 1.31 (s, 12H), 4.24 (s, 2H), 7.12-7.18 (m, 1H), 7.29-07.41 (m, 2H), 7.68 (d, J=8.4 Hz, 1H), 7.76 (s, 1H), 7.82 (t, J=8.0 Hz, 1H), 9.40 (s, 1H).

Step 3: A mixture of 3-(3,4-difluorobenzyl)-8-fluoro-7-(4,4,5,5-tetramethyl-1,3,2-dioxaborolan-2-yl)isoquinoline (0.08 g, 0.21 mmol, 1.0 eq), 5-bromo-7-cyclopropyl-7H-pyrrolo[2,3-d]pyrimidin-4-amine (0.4 g, 0.16 mmol, 0.8 eq), Pd₂(dba)₃ (10 mg, 0.01 mmol, 0.05 equiv) and K₃PO₄ (0.09 g, 0.43 mmol, 2.0 equiv) in 8 mL of Dioxane and 1.0 mL of water was bubbled with argon for 5 minutes, and then tri-(t-butyl)phosphonium tetrafluoroborate (6 mg, 0.02 mmol, 0.1 equiv) was added. The mixture was heated to 110° C. and stirred for 1 h. The reaction mixture was cooled to ambient temperature and filtered through Celite. The Celite pad was washed with 5% MeOH in DCM. The filtrate was dried over Na₂SO₄, filtered and evaporated. The crude product was purified by silica gel flash chromatography. The desired product was eluted in 2.5% MeOH in DCM. Fractions containing pure product was combined and concentrated to afford the desired product 7-cyclopropyl-5-(3-(2,3-difluorobenzyl)-8-fluoroisoquinolin-7-yl)-7H-pyrrolo[2,3-d]pyrimidin-4-amine (41 mg, 43%) as an off-white solid. LC-MS (ES) m/z=446.0 [M+H]⁺. ¹H NMR (400 MHz, DMSO-d6) 0.98-1.05 (m, 4H), 3.58-3.62 (m, 1H), 4.25 (s, 2H), 6.12 (br. s., 2H), 7.12-7.18 (m, 1H), 7.30-7.40 (m, 3H), 7.71 (t, J=8.0 Hz, 1H), 7.76-7.81 (m, 2H), 8.14 (s, 1H), 9.40 (s, 1H).

### Example 6

### 5-(3-(3,5-difluorobenzyl)-8-fluoroisoquinolin-7-yl)-7-ethyl-7H-pyrrolo[2,3-d]pyrimidin-4-amine

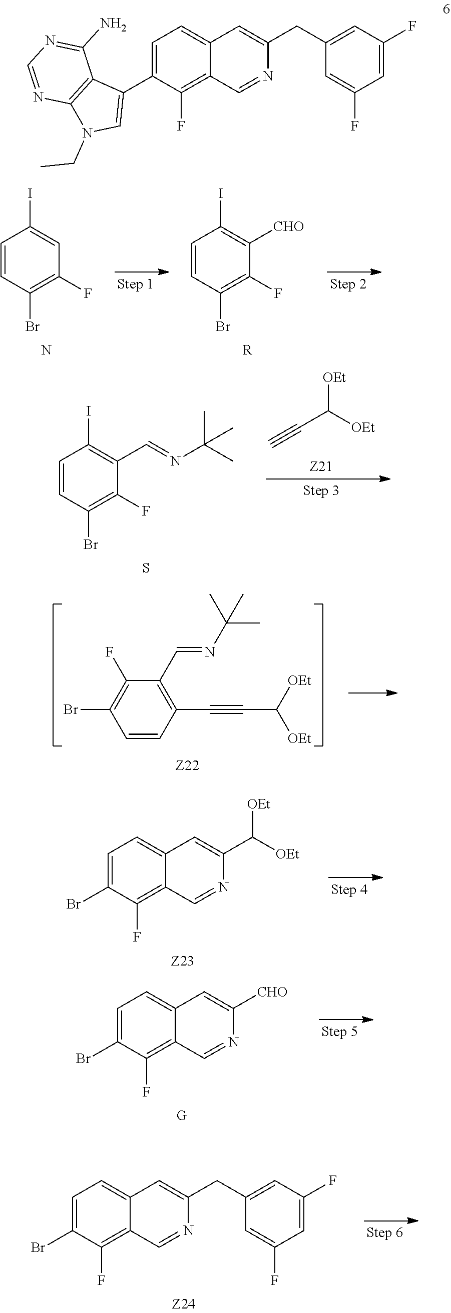

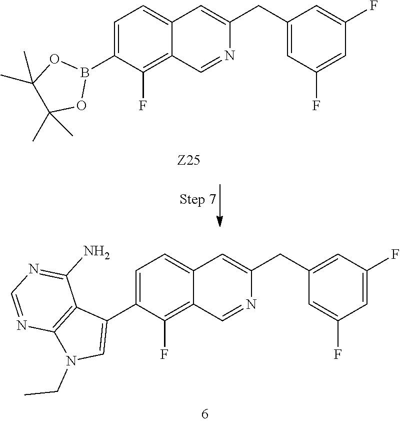 [SMILES: CC1(C)OB(c2ccc3cc(Cc4cc(F)cc(F)c4)ncc3c2F)OC1(C)C ; CCn1cc(-c2ccc3cc(Cc4cc(F)cc(F)c4)ncc3c2F)c2c(N)ncnc21]

Step 1: To a stirred solution of 1-bromo-2-fluoro-4-iodobenzene (25 g, 83.09 mmol, 1.0 equiv) in THF (300 mL) at −78° C. was added LDA (62 mL, 124.63 mmol, 1.5 equiv) (2M in THF/Heptane/Ethyl benzene) drop wise and the resulting mixture was stirred at the same temperature for 2 h. Then a solution of DMF (19.4 mL, 249.3 mmol, 3.0 equiv) in THF (20 mL) was added drop wise and stirred at the same temperature (−78° C.) for 1-2 h. After completion of the reaction, the mixture was quenched with sat.NH4Cl solution at −78° C. and allowed to reach to room temperature. The reaction mixture was diluted with EtOAc and the two layers were separated. The aqueous Phase was extracted with EtOAc (3×20 mL), and the combined organics was washed with Brine, dried over Na2SO4, filtered and evaporated to give crude product.

The reaction was performed as described 3 times, and the crude products obtained from all runs were combined and purified by silica gel column chromatography. The desired product was eluted out in 1-3% EtOAc:Hexanes. Fractions containing pure product were combined and concentrated to afford the desired product 3-bromo-2-fluoro-6-iodobenzaldehyde (Combined yield=42.81 g, 69%; Impure fractions were concentrated to give crop-2: 15 g, ˜85% pure) as a pale yellow solid. ¹H NMR (400 MHz, DMSO-d6) δ ppm 7.67 (t, J=8.0 Hz, 1H), 7.81 (d, J=8.4 Hz, 1H), 9.91 (s, 1H).

Step 2: Run1: To a stirred solution of 3-bromo-2-fluoro-6-iodobenzaldehyde (21 g, 63.85 mmol, 1.0 equiv) in water (16 mL) at 0° C. was added tert-Butyl amine (20 mL, 191.55 mmol, 3.0 equiv). The reaction mixture was then stirred at room temperature for 14 h. The reaction mixture was evaporated under reduced pressure to remove excess tert-Butyl amine. The crude reaction mixture was mixed with run 2.

Run2: To a stirred solution of 3-bromo-2-fluoro-6-iodobenzaldehyde (21 g, 63.85 mmol, 1.0 equiv) in water (16 mL) at 0° C. was added tert-Butyl amine (20 mL, 191.55 mmol, 3.0 equiv). The reaction mixture was then stirred at room temperature for 14 h. The reaction mixture was evaporated under reduced pressure to remove excess tert-Butyl amine. Combined crude mixtures from run 1 and 2 were diluted with EtOAc. The organic layer was separated, dried over Na2SO4 and filtered, and evaporated in vacuo to afford the desired compound 1-(3-bromo-2-fluoro-6-iodophenyl)-N-(tert-butyl)methanimine (48.03 g, crude) as yellow oil. ¹H NMR (400 MHz, DMSO-d6) δ ppm 1.24 (s, 9H), 7.47 (t, J=8.0 Hz, 1H), 7.68 (d, J=8.4 Hz, 1H), 8.12 (s, 1H).

Step 3: Run 1: To a stirred solution of 1-(3-bromo-2-fluoro-6-iodophenyl)-N-(tert-butyl) methanimine (24.0 g, 62.5 mmol, 1.0 equiv) in Et3N (300 mL) was added 3,3-diethoxyprop-1-yne (9.9 mL, 68.75 mmol, 1.1 equiv) and the mixture was degassed under Nitrogen for 5 min. Bis(triphenylphosphine) palladium(ii) dichloride (0.88 g, 1.25 mmol, 0.02 equiv) was added followed by CuI (0.24 g, 1.25 mmol, 0.02 equiv) and the reaction was heated to 55° C. for 2 h. The consumption of the starting material was monitored by TLC. The reaction mixture was cooled to room temperature and the precipitates were filtered off though Celite and the celite pad was washed with Ether (2×25 mL). The filtrate was dried over Na2SO4, filtered and evaporated in vacuo. The crude product was dissolved in DMF (250 mL), degassed under Nitrogen for 5 min and then CuI (1.19 g, 6.25 mmol, 0.1 equiv) was added.

The reaction mixture was heated to 100° C. for 6 h. The reaction was cooled to room temperature, diluted with EtOAc, washed with saturated NH₄Cl solution followed by brine solution, dried over Na2SO4, filtered and evaporated to give desired product (31.06 g, Crude). LC-MS (ES) m/z=328.0, 330.0 [M+H]⁺.

Run 2: To a stirred solution of 1-(3-bromo-2-fluoro-6-iodophenyl)-N-(tert-butyl) methanimine (24.0 g, 62.5 mmol, 1.0 equiv) in Et3N (300 mL) was added 3,3-diethoxyprop-1-yne (9.9 mL, 68.75 mmol, 1.1 equiv) and the mixture was degassed under Nitrogen for 5 min. Bis(triphenylphosphine) palladium(ii) dichloride (0.88 g, 1.25 mmol, 0.02 equiv) was added followed by CuI (0.24 g, 1.25 mmol, 0.02 equiv) and heated to 55° C. for 2 h. The consumption of the starting material was monitored by TLC. The reaction mixture was cooled to room temperature and the precipitates were filtered off though Celite and the celite pad was washed with Ether (2×25 mL). The filtrate was dried over Na2SO4, filtered and evaporated in vacuo. The crude product was dissolved in DMF (250 mL), degassed under Nitrogen for 5 min and then CuI (1.19 g, 6.25 mmol, 0.1 equiv) was added. The reaction mixture was heated to 100° C. for 6 h. The reaction was cooled to room temperature, diluted with EtOAc, washed with Sat. NH₄Cl solution followed by brine solution, dried over Na2SO4, filtered and evaporated to give desired product. The crude product from run 1 & run 2 were combined and purified by silica gel flash chromatography. The desired product was eluted out in 6% EtOAc:Hexanes. Fractions containing the product were combined and evaporated to afford the desired product 7-bromo-3-(diethoxymethyl)-8-fluoroisoquinoline (combined yield 25.5 g, 62%) as brown solid. LC-MS (ES) m/z=328.0, 330.0 [M+H]⁺. ¹H NMR (400 MHz, CDCl3) δ ppm 1.28 (t, J=7.2 Hz, 6H), 3.63-3.76 (m, 4H), 5.68 (s, 1H), 7.55 (d, J=8.8 Hz, 1H), 7.75-7.79 (m, 1H), 7.95 (s, 1H), 9.51 (s, 1H).

Step 4: To a stirred solution of 7-bromo-3-(diethoxymethyl)-8-fluoroisoquinoline (25.0 g, 76.18 mmol, 1.0 equiv) in Acetone:water (250 mL; 250 mL) was added p-Toluene sulfonic acid (1.32 g, 7.62 mmol, 0.1 equiv) at room temperature and the solution was heated to 80° C., stirred for 6 h. TLC showed complete conversion and the reaction mixture was evaporated to remove Acetone completely. The Aq. Phase was basified with Sat. NaHCO₃ solution and the precipitate formed was extracted with DCM (3×50 mL). The combined organic phase was washed with brine, dried over Na2SO4, filtered and evaporated to afford the desired product 7-bromo-8-fluoroisoquinoline-3-carbaldehyde as yellow solid (13.98 g, 72%). LC-MS (ES) m/z=253.9, 255.9 [M+H]⁺. ¹H NMR (400 MHz, DMSO-d6) δ ppm 8.08 (d, J=9.2 Hz, 1H), 8.15 (t, J=6.4 Hz, 1H), 8.60 (s, 1H), 9.63 (s, 1H), 10.17 (s, 1H).

Step 5: Run1: A solution of 7-bromo-8-fluoroisoquinoline-3-carbaldehyde (3.0 g, 11.81 mmol, 1.0 equiv) and 4-methylbenzenesulfonohydrazide (2.41 g, 12.99 mmol, 1.1 equiv) in 1,4-Dioxane (60 mL) was stirred at 80° C. for 2 h. Potassium phosphate (3.76 g, 17.71 mmol, 1.5 equiv) and (3,4-difluorophenyl)boronic acid (3.73 g, 23.62 mmol, 2.0 equiv) were added and heated to 110° C. and stirred for 16 h. The reaction mass was allowed to reach room temperature, and the solvent was evaporated. The crude mass was partitioned between EtOAc and sat. NaHCO₃. The two layers were separated and the aqueous phase was extracted with EtOAc (2×10 mL). The combined organic layers were washed with saturated NaHCO3, brine solution, dried over Na2SO4 and filtered. The solvent was removed under reduced pressure and the crude product was purified by Silica gel flash chromatography. The desired product was eluted in 6% EtOAc:Hex. The collected fractions with pure product were combined and concentrated in vacuo to afford the desired product 7-bromo-3-(3,5-difluorobenzyl)-8-fluoroisoquinoline (0.609 g, 15%) as pale yellow solid. LC-MS (ES) m/z=352.1, 354.1 [M+H]⁺. ¹H NMR (400 MHz, CDCl3) δ ppm 4.27 (s, 2H), 6.65-6.70 (m, 1H), 6.82 (d, J=6.4 Hz, 2H), 7.43-7.45 (m, 2H), 7.72-7.76 (m, 1H), 9.48 (s, 1H).

Run2: A solution of 7-bromo-8-fluoroisoquinoline-3-carbaldehyde (3.0 g, 11.81 mmol, 1.0 equiv) and 4-methylbenzenesulfonohydrazide (2.41 g, 12.99 mmol, 1.1 equiv) in 1,4-Dioxane (60 mL) was stirred at 80° C. for 2 h. Potassium phosphate (3.76 g, 17.71 mmol, 1.5 equiv) and (3,4-difluorophenyl)boronic acid (3.73 g, 23.62 mmol, 2.0 equiv) were added and heated to 110° C. and stirred for 16 h. The reaction was allowed to cool to room temperature, and the solvent was evaporated. The crude mass was partitioned between EtOAc and sat. NaHCO3. The two layers were separated and the aqueous phase was extracted with EtOAc (2×10 mL). The combined organic layers were washed with sat. NaHCO₃, brine solution, dried over Na2SO4 and filtered. The solvent was removed under reduced pressure and the crude product was purified by Silica gel flash chromatography. The desired product was eluted in 6% EtOAc:Hex. The collected fractions with pure product were combined and concentrated in vacuo to afford the desired product 7-bromo-3-(3,5-difluorobenzyl)-8-fluoroisoquinoline (0.52 g, 13%) as pale yellow solid. LC-MS (ES) m/z=352.1, 354.1 [M+H]⁺. ¹H NMR (400 MHz, CDCl3) δ ppm 4.27 (s, 2H), 6.65-6.70 (m, 1H), 6.82 (d, J=6.4 Hz, 2H), 7.43-7.45 (m, 2H), 7.72-7.76 (m, 1H), 9.48 (s, 1H).

Step 6: A mixture of 7-bromo-3-(3,5-difluorobenzyl)-8-fluoroisoquinoline (1.1 g, 3.12 mmol, 1.0 equiv), 4,4,4′,4′,5,5,5′,5′-octamethyl-2,2′-bi(1,3,2-dioxaborolane) (1.03 g, 4.06 mmol, 1.3 equiv), potassium acetate (0.92 g, 9.37 mmol, 3.0 equiv) and PdCl2(dppf)-CH₂Cl₂ adduct (0.13 g, 0.16 mmol, 0.05 equiv) in 40 mL of 1,4-dioxane was degassed under Argon for 5 min. and heated in an oil bath at 100° C. for 16 h. The mixture was filtered through Celite and the Celite pad was washed with DCM. The filtrate was concentrated in vacuo. The organic layer was dried over Na₂SO₄, filtered, and concentrated in vacuo. The crude product was purified by Silica gel flash chromatography. The desired product was eluted in 8% EtOAc:Hex. Fractions containing pure product was combined and concentrated to afford the desired product 3-(3,5-difluorobenzyl)-8-fluoro-7-(4,4,5,5-tetramethyl-1,3,2-dioxaborolan-2-yl)isoquinoline (0.39 g, 32%) as a white solid. Impure fractions were concentrated to give crop-2 (0.36 g, ˜60% pure). LC-MS (ES) m/z=318.1 [M+H]⁺−81. ¹H NMR (400 MHz, CDCl3) δ ppm 1.39 (s, 12H), 4.26 (s, 2H), 6.65-6.70 (m, 1H), 6.82-6.84 (m, 2H), 7.43 (s, 1H), 7.48 (d, J=8.4 Hz, 1H), 7.89-7.93 (m, 1H), 9.51 (s, 1H).

Step 7: A mixture of 3-(3,5-difluorobenzyl)-8-fluoro-7-(4,4,5,5-tetramethyl-1,3,2-dioxaborolan-2-yl)isoquinoline (0.3 g, 0.75 mmol, 1.0 equiv), 5-bromo-7-ethyl-7H-pyrrolo[2,3-d]pyrimidin-4-amine (0.145 g, 0.601 mmol, 0.8 equiv), Pd₂(dba)₃ (0.036 g, 0.04 mmol, 0.05 equiv) and K₃PO₄ (0.319 g, 1.50 mmol, 2.0 equiv) in 25 mL of Dioxane and 1.0 mL of water was degassed under Argon for 5 min, followed by addition of tri-(t-butyl)phosphonium tetrafluoroborate (0.022 g, 0.08 mmol, 0.1 equiv). The mixture was heated at 110° C. for 1 h. The reaction mixture was cooled to ambient temperature and filtered through Celite. The Celite pad was washed with 5% MeOH:DCM. The filtrate was dried over Na2SO4, filtered and evaporated. The crude product was purified by Silica gel flash chromatography. The desired product was eluted in 3% MeOH:DCM. Fractions containing pure product was combined and concentrated to afford the desired product (0.16 g, 49%) as an off-white solid. LC-MS (ES) m/z=434.2 [M+H]⁺. ¹H NMR (400 MHz, DMSO-d6) δ ppm 1.38 (t, J=7.2 Hz, 3H), 4.22 (q, J=7.2 Hz, 2H), 4.29 (s, 2H), 6.13 (br. s., 2H), 7.01-7.08 (m, 3H), 7.49 (s, 1H), 7.73 (t, J=8.0 Hz, 1H), 7.80 (d, J=8.4 Hz, 1H), 7.84 (s, 1H), 8.13 (s, 1H), 9.42 (s, 1H).

### Example 7, 8, & 9

### (7-(4-amino-7-cyclopropyl-7H-pyrrolo[2,3-d]pyrimidin-5-yl)-8-fluoroisoquinolin-3-yl)(3,5-difluorophenyl)methanol AND its enantiomers

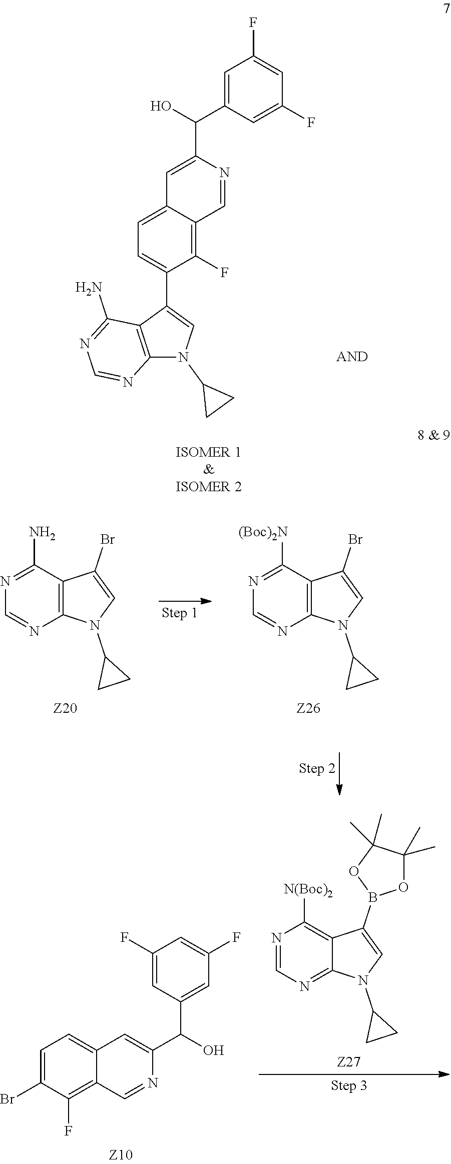

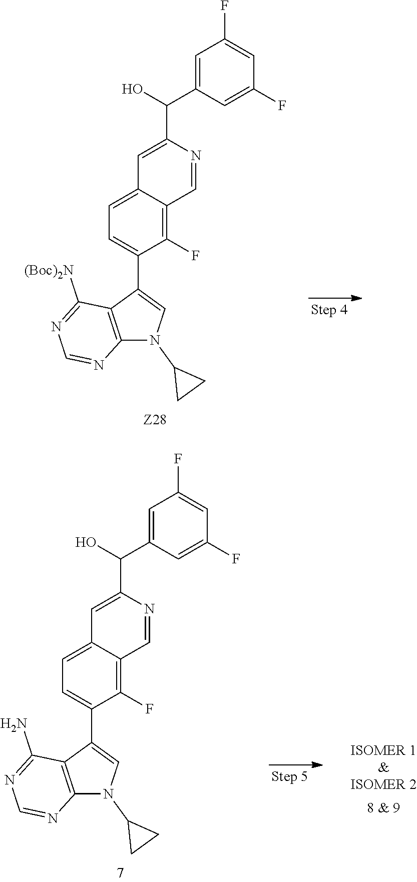

Step 1: To a stirred solution of 5-bromo-7-cyclopropyl-7H-pyrrolo [2, 3-d] pyrimidin-4-amine (3.0 g, 11.85 mmol, 1 equiv) in THF (40 mL) was added Boc anhydride (6.8 mL, 29.6 mmol, 2.5 equiv) followed by DMAP (0.3 g, 2.3 mmol, 0.2 equiv). The reaction mixture was stirred at room temperature for 24 h. Solvents were completely evaporated and the crude was extracted with ethyl acetate. The organic layer was dried over sodium sulphate, filtered and evaporated to obtain N,N-Di(tert-butoxycarbonyl)5-bromo-7-cyclopropyl-7H-pyrrolo[2,3-d]pyrimidin-4-amine (5 g, 93.1% yield). LCMS (ES) m/z=453.1, 455.10 [M+H]⁺. ¹H NMR (400 MHz, DMSO-d₆) δ ppm 1.03-1.06 (m, 4H), 1.38 (s, 9H), 1.43 (s, 9H), 3.63-3.69 (m, 1H), 7.88 (s, 1H), 8.78 (s, 1H).

Step 2: To a stirred solution of N,N-Di(tert-butoxycarbonyl)5-bromo-7-cyclopropyl-7H-pyrrolo[2,3-d]pyrimidin-4-amine (4.0 g, 8.83 mmol, 1 equiv), in 1,4-Dioxane (40 mL) was added 4,4,5,5-tetramethyl-1,3,2-dioxaborolane (5.1 mL, 35.30 mmol, 4 equiv) and triethylamine (5 mL, 35.30 mmol, 4 equiv). The reaction mixture was degassed for 5 minutes. X-Phos (0.42 g, 0.8 mmol, 0.1 equiv) followed by Pd₂(dba)₃ (0.8 g, 0.8 mmol, 0.1 equiv) were added and the reaction mixture was further degassed for 5 min. The reaction mixture was heated to 100° C. for 6 h. The reaction mixture was cooled to room temperature and completely evaporated to obtain crude compound which was purified over silica gel flash column chromatography. The compound eluted out in 50% EtOAc:Hexanes. The fractions were evaporated to obtain N,N-Di(tert-butoxycarbonyl)7-cyclopropyl-5-(4,4,5,5-tetramethyl-1,3,2-dioxaborolan-2-yl)-7H-pyrrolo[2,3-d]pyrimidin-4-amine (3.3 g, crude) as a yellow solid. LCMS (ES) m/z=401.2 [M+H]⁺−100.

Step 3: A mixture of (7-bromo-8-fluoroisoquinolin-3-yl)(3,5-difluorophenyl)methanol (0.3 g, 0.814 mmol, 1.0 equiv), N,N-Di(tert-butoxycarbonyl)7-cyclopropyl-5-(4,4,5,5-tetramethyl-1,3,2-dioxaborolan-2-yl)-7H-pyrrolo[2,3-d]pyrimidin-4-amine (0.36 g, 0.73 mmol, 0.9 equiv) and potassium phosphate (0.345 g, 1.628 mmol, 2 equiv) in 1,4-dioxane:water (16 mL: 4 mL) in multi neck round bottom flask was bubbled with N₂ for 15 min. Pd₂(dba)₃ (0.037 g, 0.040 mmol, 0.05 equiv) and tri-tert-butylphosphoniumtetrafluoroborate (0.023 g, 0.0814 mmol, 0.1 equiv) were added and heated at 100° C. for 2 h. The reaction mixture was cooled and filtered through a celite bed. The organic layer was separated and aqueous layer was extracted with EtOAc. The Combined organic layer was washed with brine solution, dried over Na₂SO₄, filtered and evaporated to obtain crude product. The crude product was purified over silica gel flash column chromatography. The compound eluted out in 2.0% MeOH:DCM. The pure fractions were evaporated to give N,N-Di(tert-butoxycarbonyl (7-(4-amino-7-cyclopropyl-7H-pyrrolo[2,3-d]pyrimidin-5-yl)-8-fluoroisoquinolin-3-yl)(3,5-difluorophenyl)methanol (0.4 g, Crude) as gummy compound. LCMS (ES) m/z=662.2 [M+H]⁺.

Step 4: To a stirred solution of N,N-Di(tert-butoxycarbonyl (7-(4-amino-7-cyclopropyl-7H-pyrrolo[2,3-d]pyrimidin-5-yl)-8-fluoroisoquinolin-3-yl)(3,5-difluorophenyl)methanol (0.4 g, 0.60 mmol, 1 eq) in DCM (10 mL) was added Triflouoro acetic (4 mL) drop wise at −0° C. The reaction mixture was stirred at room temperature for 3 h. After completion of the reaction, the mixture was evaporated, and the residue was dissolved in DCM, and washed with saturated Sodium bicarbonate solution. The organic layer was dried over Na₂SO₄, filtered and evaporated to obtain crude product. The crude product was purified over silica gel flash column chromatography. The compound eluted out in 5% MeOH:DCM. The product was further purified by preparative HPLC. Condition:Column:Intersill ODA 3V (250 mm×20 mm×5 mic), Mobile phase (A): 0.1% Ammonia in water, Mobile phase (B): ACN, Flow rate 19 mL/min. The pure fractions were evaporated to obtain (7-(4-amino-7-cyclopropyl-7H-pyrrolo[2,3-d]pyrimidin-5-yl)-8-fluoroisoquinolin-3-yl)(3,5-difluorophenyl)methanol (0.038 g, 14%) as white solid. LCMS (ES) m/z=462.1 [M+H]⁺. ¹H NMR (400 MHz, DMSO-d6) δ ppm 1.04-1.05 (m, 4H), 3.59 (bs, 1H), 5.94 (d, J=4.0 Hz, 1H), 6.13 (bs, 2H), 6.47 (d, J=4.0 Hz, 1H), 7.04 (s, 1H), 7.14 (d, J=6.8 Hz, 2H), 7.34 (s, 1H), 7.73 (t, J=7.8 Hz, 1H), 7.89 (d, J=8.4 Hz, 1H), 8.09 (s, 1H), 8.14 (s, 1H), 9.37 (s, 1H). HPLC: 99.56% purity by HPLC @242 nM.

Step 5: Chiral separation of Isomers:

0.132 g of Racemic compound (7-(4-amino-7-cyclopropyl-7H-pyrrolo[2,3-d]pyrimidin-5-yl)-8-fluoroisoquinolin-3-yl)(3,5-difluorophenyl)methanol was separated by chiral preparative HPLC Conditions: Column: CHIRALPAK IC (250 mm×20 mm×5 mic); Mobile Phase: n-Hexane:EtOH with 0.1% DEA (50:50); Flow rate: 15.0 mL/min. Pure fractions at retention time 8.67 min were concentrated to obtain enantiomer 1 as off white solid (0.026 g, 39% yield). LCMS (ES) m/z=462.1 [M+H]⁺. ¹H NMR (400 MHz, DMSO-d6) δ ppm 0.99-1.05 (m, 4H), 3.58-3.60 (m, 1H), 5.94 (d, J=4.0 Hz, 1H), 6.13 (br. s., 2H), 6.47 (d, J=4.4 Hz, 1H), 7.04 (t, J=9.2 Hz, 1H), 7.14 (d, J=7.2 Hz, 2H), 7.34 (s, 1H), 7.73 (t, J=7.2 Hz, 1H), 7.89 (d, J=8.4 Hz, 1H), 8.09 (s, 1H), 8.14 (s, 1H), 9.37 (s, 1H): HPLC Analytical conditions: Column: CHIRALPAK IC (250 mm×4.6 mm×5 mic); Mobile Phase: n-Hexane:EtOH with 0.1% DEA (50:50); Flow rate: 1.0 mL/min; 99.99% purity, retention time 5.965 min @282 nm. Pure fractions at retention time 11.423 min were concentrated to obtain enantiomer 2 as off white solid (0.028 g, 42% yield). LCMS (ES) m/z=462.1 [M+H]⁺. ¹H NMR (400 MHz, DMSO-d6) δ ppm 0.99-1.05 (m, 4H), 3.56-3.62 (m, 1H), 5.94 (d, J=4.0 Hz, 1H), 6.13 (br. s., 2H), 6.47 (d, J=4.4 Hz, 1H), 7.04 (t, J=9.2 Hz, 1H), 7.14 (d, J=7.2 Hz, 2H), 7.34 (s, 1H), 7.73 (t, J=7.2 Hz, 2H), 7.89 (d, J=8.4 Hz, 1H), 8.09 (s, 1H), 8.14 (s, 1H), 9.37 (s, 1H): HPLC Analytical conditions: Column: CHIRALPAK IC (250 mm×4.6 mm×5 mic); Mobile Phase: n-Hexane:EtOH with 0.1% DEA (50:50); Flow rate: 1.0 mL/min; 98.53% purity, retention time 6.432 min (6.028 min, 1.47% eantiomer1) @282 nm

### Example 10

### 7-cyclopropyl-5-(3-(3,5-difluorobenzyl)-5-fluoroisoquinolin-7-yl)-7H-pyrrolo [2,3-d]pyrimidin-4-amine

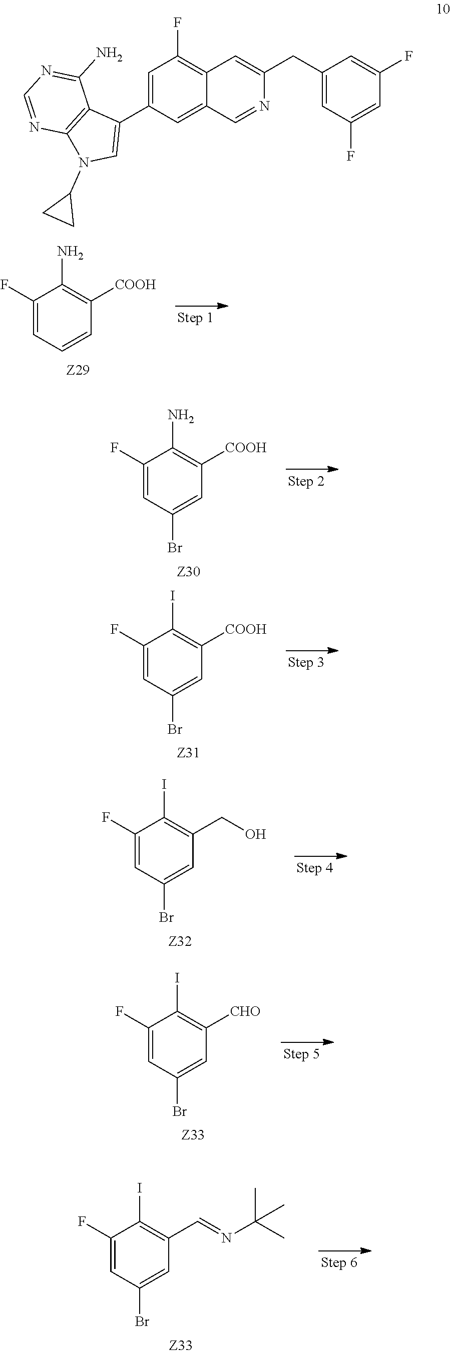

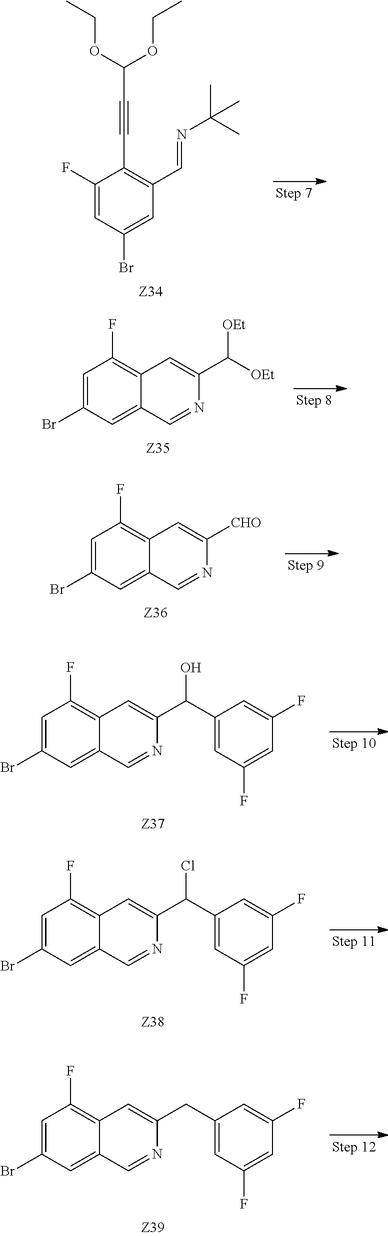 [SMILES: CCOC(C#Cc1c(F)cc(Br)cc1/C=N/C(C)(C)C)OCC ; CCOC(OCC)c1cc2c(F)cc(Br)cc2cn1 ; O=Cc1cc2c(F)cc(Br)cc2cn1 ; OC(c1cc(F)cc(F)c1)c1cc2c(F)cc(Br)cc2cn1 ; Fc1cc(F)cc(C(Cl)c2cc3c(F)cc(Br)cc3cn2)c1 ; Fc1cc(F)cc(Cc2cc3c(F)cc(Br)cc3cn2)c1]

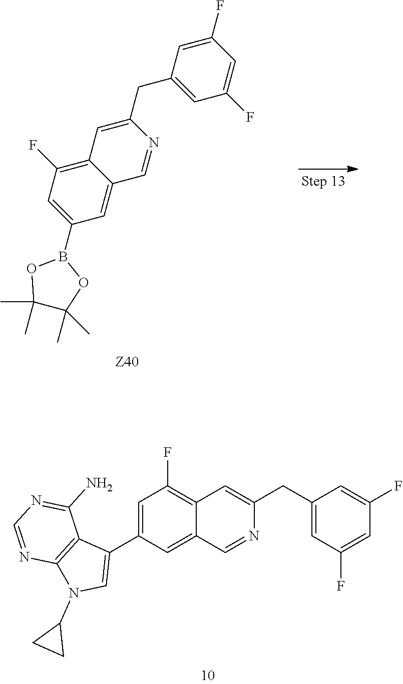 [SMILES: CC1(C)OB(c2cc(F)c3cc(Cc4cc(F)cc(F)c4)ncc3c2)OC1(C)C ; Nc1ncnc2c1c(-c1cc(F)c3cc(Cc4cc(F)cc(F)c4)ncc3c1)cn2C1CC1]

Step 1: Run 1; To a stirred solution of 2-amino-3-fluorobenzoic acid (1.0 g, 6.45 mmol, 1.0 equiv) in chloroform (10 mL) was added bromine (0.36 mL, 70.9 mmol, 1.1 equiv) in chloroform in a dropwise manner at 0° C. The reaction mixture was gradually allowed to warm to room temperature and stirred overnight. The precipitated solid was filtered under vacuum. The residue was thoroughly washed with DCM and dried under vacuum to obtain 2-amino-5-bromo-3-fluorobenzoic acid hydro bromide as an off-white solid (2.5 g crude).

LCMS (ES) m/z=234.1, 236.1 [M+H]⁺. ¹H NMR (400 MHz, DMSO-d₆) δ ppm 4.8-6.4 (br.s), 7.48-7.51 (m, 1H), 7.61 (s, 1H).

Run 2; Toa stirred solution of 2-amino-3-fluorobenzoic acid (6.9 g, 44.5 mmol, 1.0 equiv) in chloroform (70 mL) was added bromine (2.5 mL, 48.96 mmol, 1.1 equiv) in chloroform in a dropwise manner at 0° C. The reaction mixture was gradually allowed to warm to room temperature and stirred overnight. The precipitated solid was filtered under vacuum. The residue was thoroughly washed with DCM and dried under vacuum to obtain 2-amino-5-bromo-3-fluorobenzoic acid hydro bromide as an off-white solid (12 g crude). LCMS (ES) m/z=233.9, 235.9 [M+H]⁺. ¹H NMR (400 MHz, DMSO-d₆) δ ppm 5.8-6.8 (br.s), 7.46-7.49 (m, 1H), 7.60 (s, 1H).

Step 2: Run 1; To a stirred solution of 2-amino-5-bromo-3-fluorobenzoic acid hydro bromide (2.5 g, 7.98 mmol, 1.0 equiv) in sulphuric acid (2 mL) was added HCl (2 mL) at 0° C. Sodium nitrite (0.55 g, 7.98 mmol, 1 equiv) in water (7 mL) was added in a dropwise manner and stirred for 1 hour at the same temperature. Potassium iodide (2.65 g, 15.97 mmol, 2 equiv) in water (8 mL) was added and stirred for further 3 hours at room temperature. The reaction mixture was filtered under vacuum. The residue was thoroughly washed with water and dried under vacuum to obtain 5-bromo-3-fluoro-2-iodobenzoic acid (0.8 g, 30%) as brown solid. ¹H NMR (400 MHz, DMSO-d₆) δ ppm 7.65 (s, 1H), 7.71-7.73 (m, 1H), 13.77 (s, 1H).

Run 2: To a stirred solution of 2-amino-5-bromo-3-fluorobenzoic acid hydro bromide (12 g, 38.33 mmol, 1.0 equiv) in sulphuric acid (12 mL) was added HCl (12 mL) at 0° C. Sodium nitrite (0.55 g, 38.33 mmol, 1 equiv) in water (10 mL) was added in a dropwise manner and stirred for 1 hour at the same temperature. Potassium iodide (12.72 g, 76.67 mmol, 2 equiv) in water (10 mL) was added to it and stirred for further 3 hours at room temperature. The reaction mixture was filtered under vacuum. The residue was thoroughly washed with water and dried under vacuum to obtain 5-bromo-3-fluoro-2-iodobenzoic acid (6.5 g crude) as a yellow solid. LCMS (ES) m/z=344.9, 346.9 [M+H]⁺.

Step 3: Run 1; To a stirred solution of 5-bromo-3-fluoro-2-iodobenzoic acid (0.8 g, 2.32 mmol, 1.0 equiv), in THF (15 mL) was added borane-dimethyl sulfide complex (1.1 mL, 11.6 mmol, 5 equiv) at 0° C. The reaction mixture was warmed to room temperature and stirred for overnight. The reaction mixture was quenched with methanol in a dropwise manner and completely evaporated to obtain crude (5-bromo-3-fluoro-2-iodophenyl) methanol (0.6 g crude) as off-white solid. ¹H NMR (400 MHz, DMSO-d₆) δ ppm 4.42 (d, J=5.6 Hz, 2H), 5.64 (t, J=6.0 Hz), 7.42 (s, 1H), 7.46-7.48 (m, 1H).

Run 2: To a stirred solution of 5-bromo-3-fluoro-2-iodobenzoic acid (6 g, 17.44 mmol, 1.0 equiv), in THF (50 mL) was added borane-dimethyl sulfide complex (6.6 mL, 87.2 mmol, 5 equiv) at 0° C. The reaction mixture was warmed to room temperature and stirred for overnight. The reaction mixture was quenched with methanol in a dropwise manner and completely evaporated to obtain crude (5-bromo-3-fluoro-2-iodophenyl)methanol (3.8 g, 66.6%) as an off-white solid. ¹H NMR (400 MHz, DMSO-d₆) δ ppm 4.42 (d, J=5.6 Hz, 2H), 5.64 (t, J=6.0 Hz), 7.42 (s, 1H), 7.45-7.47 (m, 1H).

Step 4: Run 1; To a stirred solution of (5-bromo-3-fluoro-2-iodophenyl)methanol (0.2 g, 0.606 mmol, 1.0 equiv), in DCM (10 mL) was added Manganese dioxide (0.37 g, 4.24 mmol, 7 equiv) at room temperature and stirred for 24 hours. The reaction mixture was filtered through celite and the filtrate was completely evaporated to obtain 5-bromo-3-fluoro-2-iodobenzaldehyde (0.16 g, 80.8%) as an off-white solid. ¹H NMR (400 MHz, DMSO-d₆) b ppm 7.72 (s, 1H), 7.92-7.93 (m, 1H), 9.91 (s, 1H).

Run 2: To a stirred solution of (5-bromo-3-fluoro-2-iodophenyl)methanol (3.6 g, 10.9 mmol, 1.0 equiv), in DCM (40 mL) was added Manganese dioxide (6.6 g, 76.3 mmol, 7 equiv) at room temperature and stirred for 24 hours. The reaction mixture was filtered through celite and the filtrate was completely evaporated to obtain 5-bromo-3-fluoro-2-iodobenzaldehyde (3.3 g, 92%) as an off-white solid. ¹H NMR (400 MHz, DMSO-d₆) δ ppm 7.72 (s, 1H), 7.92-7.93 (m, 1H), 9.91 (s, 1H).

Step 5: Run 1; To a stirred solution of 5-bromo-3-fluoro-2-iodobenzaldehyde (0.16 g, 0.48 mmol, 1.0 equiv) in water (0.12 mL) was added 2-methylpropan-2-amine (0.16 mL, 1.46 mmol, 3 equiv) at room temperature and stirred for 12 hours. Solvents were completely evaporated and the crude was extracted with ethyl acetate. The organic layer was dried over sodium sulphate and evaporated to obtain crude 1-(5-bromo-3-fluoro-2-iodophenyl)-N-(tert-butyl) methanimine (0.2 g crude) as an oily compound. ¹H NMR (400 MHz, DMSO-d₆) δ ppm 1.25 (s, 9H), 7.68 (d, J=7.6 Hz, 1H), 7.74 (s, 1H), 8.33 (s, 1H).

Run 2: To a stirred solution of 5-bromo-3-fluoro-2-iodobenzaldehyde (2.8 g, 8.5 mmol, 1.0 equiv) in water (2.1 mL) was added 2-methylpropan-2-amine (2.7 mL, 25.6 mmol, 3 equiv) at room temperature and stirred for 12 hours. Solvents were completely evaporated and the crude was extracted with ethyl acetate. The organic layer was dried over sodium sulphate and evaporated off to obtain crude 1-(5-bromo-3-fluoro-2-iodophenyl)-N-(tert-butyl) methanimine (3 g crude) as an oily compound. ¹H NMR (400 MHz, DMSO-d₆) δ ppm 1.25 (s, 9H), 7.65-7.68 (m, 1H), 7.73-7.74 (m, 1H), 8.34 (s, 1H).

Step 6: To a stirred solution of 1-(5-bromo-3-fluoro-2-iodophenyl)-N-(tert-butyl) methanimine (3 g, 7.8 mmol, 1.0 equiv) in Triethylamine (20 mL) was added (1.2 g, 9.3 mmol, 1.2 equiv) of 3,3-diethoxyprop-1-yne. The reaction mixture was purged with N₂ gas and Bis(triphenylphosphine) palladium(II)dichloride (0.11 g, 0.156 mmol, 0.02 equiv) followed by copper iodide (0.03 g, 0.156 mmol, 0.02 equiv) were added. The reaction mixture was further purged with N2 gas and heated to 55° C. for 2 hours. The reaction mixture was cooled to room temperature and filtered through celite. The filtrate was completely evaporated to obtain crude 1-(5-bromo-2-(3, 3-diethoxyprop-1-yn-1-yl)-3-fluorophenyl)-N-(tert-butyl) methanimine (3.0 g) as a gummy solid. ¹H NMR (400 MHz, DMSO-d₆) δ ppm 1.15 (t, J=6.8 Hz, 6H), 1.23 (s, 9H), 3.57-3.61 (m, 2H), 3.66-3.70 (m, 2H), 5.63 (s, 1H), 7.78 (d, J=8.0 Hz, 1H), 7.87 (s, 1H), 8.53 (s, 1H).

Step 7: To a stirred solution of 1-(5-bromo-2-(3,3-diethoxyprop-1-yn-1-yl)-3-fluorophenyl)-N-(tert-butyl)methanimine (3 g, 7.8 mmol, 1.0 equiv) in DMF was added copper iodide (0.15 g, 0.78 mmol, 0.1 equiv). The reaction mixture was heated to 100° C. for 6 hours. The reaction mixture was cooled to room temperature and filtered through celite. The filtrate was treated with water and extracted in ethyl acetate. The organic layer was dried over sodium sulphate and evaporated to obtain crude which was purified over silica gel flash column chromatography. The compound eluted out in 30% EtOAc:Hexanes. The pure fractions were evaporated to obtain 7-bromo-3-(diethoxymethyl)-5-fluoroisoquinoline (1.4 g, 56%) as an off-white solid. ¹H NMR (400 MHz, DMSO-d₆) δ ppm 1.15 (t, J=7.2 Hz, 6H), 3.60 (q, J=7.2 Hz, 4H), 5.60 (s, 1H), 7.91-7.94 (m, 2H), 8.32 (s, 1H), 9.35 (s, 1H).

Step 8: To a stirred solution of 7-bromo-3-(diethoxymethyl)-5-fluoroisoquinoline (1.4 g, 4.26 mmol, 1.0 equiv) in acetone:water (10 mL:10 mL) was added p-toluenesulfonic acid (0.08 g, 0.426 mmol, 0.1 equiv) at room temperature. The reaction mixture was heated to 80° C. for 12 hours. The reaction mixture was cooled to room temperature and the solvents were evaporated. The reaction mixture was neutralized with saturated NaHCO₃ solution and extracted in DCM. The organic layer was dried over sodium sulphate and evaporated to obtain crude compound which was triturated in diethyl ether. The precipitated solid was filtered and dried under vacuum to obtain 7-bromo-5-fluoroisoquinoline-3-carbaldehyde (0.8 g, 74%) as brown solid. LCMS (ES) m/z=254.0, 256.0 [M+H]⁺. ¹H NMR (400 MHz, DMSO-d₆) δ ppm 8.08-8.11 (m, 1H), 8.45-8.46 (m, 2H), 9.54 (s, 1H), 10.16 (s, 1H).

Step 9: Run 1; To a stirred solution of 7-bromo-5-fluoroisoquinoline-3-carbaldehyde (0.05 g, 0.196 mmol, and 1.0 equiv) in THF (5 mL) was added 3, 5-difluoro phenyl magnesium bromide (0.5 M in THF) (0.6 mL, 1.5 equiv) in a dropwise manner at room temperature and heated to 50° C. for 12 hours. The reaction mixture was cooled to room temperature and quenched with saturated ammonium chloride solution. The crude was extracted in ethyl acetate. The organic layer was dried over sodium sulphate and evaporated off to obtain oily compound which was purified over silica gel flash column chromatography. The compound eluted out in 30% EtOAc:Hexanes. The pure fractions were evaporated to obtain (7-bromo-5-fluoroisoquinolin-3-yl)(3,5-difluorophenyl)methanol (0.025 g, 35%) as an oily compound. LCMS (ES) m/z=368.0, 370.0 [M+H]⁺. ¹H NMR (400 MHz, DMSO-d₆) δ ppm 5.91 (d, J=4.8 Hz, 1H), 6.49 (d, J=4.8 Hz, 1H), 7.02-7.06 (m, 1H), 7.12-7.14 (m, 2H), 7.91 (d, J=9.6 Hz, 1H), 8.05 (s, 1H), 8.26 (s, 1H), 9.28 (s, 1H).

Run 2; To a stirred solution of 7-bromo-5-fluoroisoquinoline-3-carbaldehyde (0.65 g, 2.56 mmol, and 1.0 equiv) in THF (25 mL) was added 3, 5 difluoro phenyl magnesium bromide (0.5 M in THF) (7.7 mL, 1.5 equiv) in a dropwise manner at room temperature and heated to 50° C. for 12 hours. The reaction mixture was cooled to room temperature and quenched with Sat. ammonium chloride solution. The crude was extracted in ethyl acetate. The organic layer was dried over sodium sulphate and evaporated off to obtain oily compound which was purified over silica gel flash column chromatography. The compound eluted out in 30% EtOAc:Hexanes. The fractions were evaporated to obtain (7-bromo-5-fluoroisoquinolin-3-yl) (3, 5-difluorophenyl) methanol (0.55 g crude) as an oily compound. LCMS (ES) m/z=368.0, 370.0 [M+H]⁺. ¹H NMR (400 MHz, DMSO-d₆) δ ppm 5.91 (d, J=4.8 Hz, 1H), 6.49 (d, J=4.8 Hz, 1H), 7.02-7.06 (m, 1H), 7.12-7.14 (m, 2H), 7.89-7.92 (m, 1H), 8.05 (s, 1H), 8.25 (s, 1H), 9.28 (s, 1H).

Step 10: Run 1; To a stirred solution of (7-bromo-5-fluoroisoquinolin-3-yl) (3, 5-difluorophenyl) methanol (0.025 g, 0.06 mmol, 1.0 equiv) in DCM (5 mL) was added thionyl chloride (5 mL) in a dropwise manner at 0° C. The reaction mixture was warmed to room temperature and stirred for 2 hours. Solvents were completely evaporated and the crude was triturated with n-pentane. The precipitated solid was filtered and dried under vacuum to obtain 7-bromo-3-(chloro (3, 5-difluorophenyl) methyl)-5-fluoroisoquinoline (0.02 g crude) as a brown solid. LCMS (ES) m/z=385.9, 387.9 [M+H]⁺. ¹H NMR (400 MHz, DMSO-d₆) b ppm 6.72 (s, 1H), 7.18-7.23 (m, 1H), 7.34-7.35 (m, 2H), 7.98 (d, J=10 Hz, 1H), 8.15 (s, 1H), 8.31 (s, 1H), 9.38 (s, 1H).

Run 2; To a stirred solution of (7-bromo-5-fluoroisoquinolin-3-yl) (3, 5-difluorophenyl) methanol (0.55 g, 1.49 mmol, 1.0 equiv) in DCM (10 mL) was added thionyl chloride (10 mL) in a dropwise manner at 0° C. The reaction mixture was warmed to room temperature and stirred for 2 hours. Solvents were completely evaporated and the crude was triturated with n-pentane. The precipitated solid was filtered and dried under vacuum to obtain 7-bromo-3-(chloro (3, 5-difluorophenyl) methyl)-5-fluoroisoquinoline (0.58 g crude) as a brown solid. LCMS (ES) m/z=386.0, 388.0 [M+H]⁺. ¹H NMR (400 MHz, DMSO-d₆) δ ppm 6.72 (s, 1H), 7.21 (t, J=8.8 Hz, 1H), 7.34-7.35 (m, 2H), 7.98 (d, J=9.2 Hz, 1H), 8.15 (s, 1H), 8.31 (s, 1H), 9.38 (s, 1H).

Step 11: Run 1; To a stirred solution of 7-bromo-3-(chloro(3,5-difluorophenyl)methyl)-5-fluoroisoquinoline (0.02 g, 0.05 mmol, 1.0 equiv) in MeOH (5 mL) was added Zinc metal dust—325 mesh (0.007 g, 0.05 mmol, 2.0 equiv) followed by Ammonium chloride (0.006 mg, 0.05 mmol, 2.0 equiv) at 0° C. The reaction mixture was warmed to room temperature and stirred for 2 hours. The reaction mixture was filtered through celite and the filtrate was completely evaporated to obtain crude 7-bromo-3-(3,5-difluorobenzyl)-5-fluoroisoquinoline (0.02 g crude) as a gummy solid. LCMS (ES) m/z=352.0, 354.0 [M+H]⁺. ¹H NMR (400 MHz, DMSO-d₆) δ ppm 4.28 (s, 2H), 7.01-7.06 (m, 3H), 7.85-7.93 (m, 2H), 8.26 (s, 1H), 9.31 (s, 1H).

Run 2; To a stirred solution of 7-bromo-3-(chloro(3,5-difluorophenyl)methyl)-5-fluoroisoquinoline (0.58 g, 1.5 mmol, 1.0 equiv) in MeOH (20 mL) was added Zinc metal dust—325 mesh (0.3 g, 4.5 mmol, 3.0 equiv) followed by Ammonium chloride (0.24 mg, 4.5 mmol, 3.0 equiv) at 0° C. The reaction mixture was warmed to room temperature and stirred for 2 hours. The reaction mixture was filtered through celite and the filtrate was completely evaporated to obtain 7-bromo-3-(3,5-difluorobenzyl)-5-fluoroisoquinoline (0.26 g, 50%) as an off-white solid. LCMS (ES) m/z=352.0, 354.0 [M+H]. ¹H NMR (400 MHz, DMSO-d₆) δ ppm 4.28 (s, 2H), 7.01-7.06 (m, 3H), 7.85 (s, 1H), 7.87-7.89 (m, 1H), 8.25 (s, 1H), 9.30 (s, 1H).

Step 12: Run 1; To a stirred solution of 7-bromo-3-(3,5-difluorobenzyl)-5-fluoroisoquinoline (0.03 g, 0.08 mmol, 1.0 equiv) in 1,4-Dioxane was added Bis(pinacolato)diboron (0.025 g, 0.093 mmol, 1.1 equiv) and Potassium acetate (0.025 g, 0.255 mmol, 3.0 equiv). The reaction mixture was purged with N₂ for 5 minutes. PdCl₂(dppf)DCM (0.007 g, 0.008 mmol, 0.1 equiv) was added and the reaction mixture was further purged with N2 for 5 minutes and heated to 100° C. for 2 hours. The reaction mixture was cooled to room temperature and solvents were completely evaporated and the obtained crude was purified over silica gel flash column chromatography. The compound eluted out in 30% EtOAc:Hexanes. Fractions were evaporated to obtain crude 3-(3, 5-difluorobenzyl)-5-fluoro-7-(4, 4, 5, 5-tetramethyl-1, 3, 2-dioxaborolan-2-yl) isoquinoline (0.03 g crude) as a gummy solid. LCMS (ES) m/z=400.1 [M+H].

Run 2; To a stirred solution of 7-bromo-3-(3,5-difluorobenzyl)-5-fluoroisoquinoline (0.23 g, 0.65 mmol, 1.0 equiv) in 1,4-Dioxane was added Bis(pinacolato)diboron (0.18 g, 0.718 mmol, 1.1 equiv) and Potassium acetate (0.19 g, 1.96 mmol, 3.0 equiv). The reaction mixture was purged with N₂ for 5 minutes. PdCl₂(dppf)DCM (0.53 g, 0.065 mmol, 0.1 equiv) was added and the reaction mixture was further purged with N2 for 5 minutes and heated to 100° C. for 2 hours. The reaction mixture was cooled to room temperature and solvents were completely evaporated and the obtained crude was purified over silica gel flash column chromatography. The compound eluted out in 30% EtOAc:Hexanes. Fractions were evaporated off to obtain 3-(3, 5-difluorobenzyl)-5-fluoro-7-(4, 4, 5, 5-tetramethyl-1, 3, 2-dioxaborolan-2-yl) isoquinoline (0.14 g, 48.2%) as a gummy solid. LCMS (ES) m/z=400.1 [M+H]. ¹H NMR (400 MHz, DMSO-d₆) δ ppm 1.32 (s, 12H), 4.29 (s, 2H), 7.01-7.07 (m, 3H), 7.58 (d, J=10.0 Hz, 1H), 7.89 (s, 1H), 8.31 (s, 1H), 9.43 (s, 1H).

Step 13: To a stirred solution of 3-(3,5-difluorobenzyl)-5-fluoro-7-(4,4,5,5-tetramethyl-1,3,2-dioxaborolan-2-yl)isoquinoline (0.14 g, 0.35 mmol, 1.0 equiv) in 1,4-Dioxane:H₂O (18 mL:6 mL) was added 5-bromo-7-cyclopropyl-7H-pyrrolo[2,3-d]pyrimidin-4-amine (0.062 g, 0.024 mmol, 0.7 equiv) and potassium phosphate (0.15 g, 0.07 mmol, 2.0 equiv). The reaction mixture was purged with N2 for 5 minutes and Pd₂(dba)₃ (0.016 g, 0.017 mmol, 0.05 equiv) followed by P(t-Bu)₃HBF₄ (0.010 g, 0.035 mmol, 0.1 equiv). The reaction mixture was further purged with N2 for 5 minutes and heated to 100° C. for 1 hour. The reaction mixture was cooled to room temperature and the solvents were completely evaporated to obtain crude product which was purified by silica gel flash column chromatography. The compound eluted out in 3% MeOH:DCM. The pure fractions were evaporated to obtain 7-cyclopropyl-5-(3-(3, 5-difluorobenzyl)-5-fluoroisoquinolin-7-yl)-7H-pyrrolo [2, 3-d]pyrimidin-4-amine (0.06 g, 38.4%) as an off-white solid. LCMS (ES) m/z=446.2 [M+H]⁺. ¹H NMR (400 MHz, DMSO-d₆) δ ppm 1.01-1.08 (m, 4H), 3.58-3.62 (m, 1H), 4.29 (s, 2H), 6.26 (br.s, 2H), 7.01-7.06 (m, 3H), 7.46 (s, 1H), 7.69 (d, J=10.8 Hz, 1H), 7.86 (s, 1H), 7.92 (s, 1H), 8.17 (s, 1H), 9.31 (s, 1H).

### Example 11

### 5-(3-(3,5-difluorobenzyl)-8-fluoroisoquinolin-7-yl)-7-(2,2-difluorocyclopropyl)-7H-pyrrolo[2,3-d]pyrimidin-4-amine

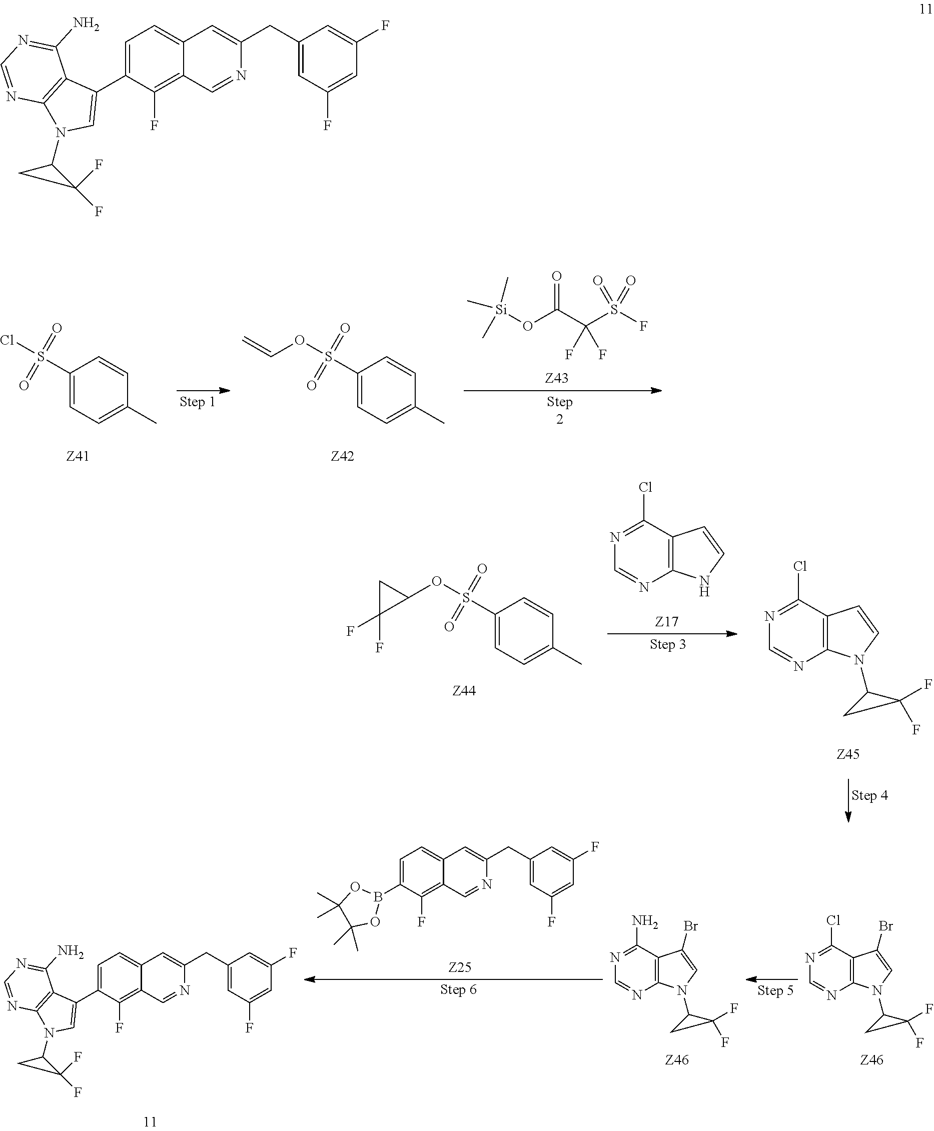

Step 1: To a stirred solution of THF (50 mL) at room temperature was added n-BuLi dropwise over a period of 10 min. The resulted yellow solution was stirred at room temperature for 3 h. The above solution was cooled to −78° C. and added 4-methylbenzenesulfonyl chloride (6.0 g, 31.57 mmole, 1.0 equiv) in THF (30 mL) dropwise at −78° C. over a period of 10 min. Reaction mixture was stirred for 30 min at −78° C. The reaction mixture was warmed to room temperature slowly and stirred another 30 min at room temperature. The reaction mixture was quenched with NH₄Cl solution and extracted with EtOAc. The organic layer was dried over sodium sulphate and evaporated to obtain crude product. The crude product was purified over silica gel flash column chromatography. The compound eluted out in 10% EtOAc in n-hexane to afford vinyl 4-methylbenzenesulfonate (2.0 g, 32%) as colourless liquid. ¹H NMR (400 MHz, CDCl₃) δ ppm−2.45 (s, 3H), 4.66-4.68 (m, 1H), 4.86-4.90 (m, 1H), 6.57-6.62 (m, 1H), 7.33-7.36 (m, 2H), 7.80 (d, J=8.0 Hz, 2H)

Step 2: To a mixture of vinyl 4-methylbenzenesulfonate (1.1 g, 5.55 mmol, 1.0 equiv), Sodium Fluoride (0.023 g, 0.55 mmol, 0.1 equiv) and xylene (0.5 mL, 0.5 V) was added trimethylsilyl 2,2-difluoro-2-(fluorosulfonyl)acetate (8.3 g, 33.3 mmol, 6 equiv) dropwise over a period of 15 min at 120° C. The reaction mixture was stirred at 120° C. for 2 h. The reaction mixture was cooled to room temperature and purified over silica gel flash column chromatography. The compound eluted out in 10% EtOAc in n-hexane to afford 2,2-difluorocyclopropyl 4-methylbenzenesulfonate (0.85 g, crude) as pale brown liquid. ¹H NMR (400 MHz, CDCl₃) δ ppm−1.58-1.67 (m, 2H), 2.47 (s, 3H), 4.21-4.27 (m, 1H), 7.36-7.38 (m, 2H), 7.81-7.84 (m, 2H).

Step 3: To a stirred solution of 4-chloro-7H-pyrrolo[2,3-d]pyrimidine (0.52 g, 3.42 mmol, 1.0 equiv), in DMF (15 mL) was added 60% sodium hydride (0.15 g, 3.76 mmol, 1.1 equiv) at 0° C. and stirred for 15 min at same temperature. 2,2-difluorocyclopropyl 4-methylbenzenesulfonate (0.85 g, 3.42 mmol, 1.0 equiv) in DMF (3 mL) was added to the reaction mixture at 0° C. The reaction mixture was warmed to room temperature and stirred for 2 h. The reaction mixture was quenched with ice water. The crude was extracted with ethyl acetate. The organic layer was dried over sodium sulphate and evaporated to obtain crude which was purified over silica gel flash column chromatography. The compound eluted out in 10% EtOAc in n-hexane to afford 4-chloro-7-(2,2-difluorocyclopropyl)-7H-pyrrolo[2,3-d]pyrimidine (0.1 g, 13%) as pale yellow solid. LCMS (ES) m/z=230.0 [M+H]⁺. ¹H NMR (400 MHz, DMSO-d₆) δ ppm −2.39 (m, 2H), 4.39-4.46 (m, 1H), 6.70 (d, J=3.2 Hz, 1H), 7.76 (d, J=3.6 Hz, 1H), 8.69 (s, 1H).

Step 4: To a stirred solution of 4-chloro-7-(2,2-difluorocyclopropyl)-7H-pyrrolo[2,3-d]pyrimidine (0.1 g, 0.43 mmol, 1 equiv) in DCM (5 mL) was added NBS (0.077 g, 0.43 mmol, 1.0 equiv) at 0° C. The reaction mixture was warmed to room temperature and stirred for 2 h. The reaction mixture was quenched with water and extracted with ethyl acetate. The organic layer was dried over sodium sulphate and evaporated to obtain 5-bromo-4-chloro-7-(2,2-difluorocyclopropyl)-7H-pyrrolo[2,3-d]pyrimidine (0.11 g, 82%) as pale yellow solid. LCMS (ES) m/z=308.0, 310.0 [M+H.]⁺. ¹H NMR (400 MHz, DMSO-d₆) δ ppm−2.30-2.55 (m, 2H), 4.39-4.45 (m, 1H), 8.07 (s, 1H), 8.73 (s, 1H)

Step 5: To a stirred solution of 5-bromo-4-chloro-7-(2,2-difluorocyclopropyl)-7H-pyrrolo[2,3-d]pyrimidine (0.1 g, 0.32 mmol, 1 equiv) in 1,4-Dioxane (5 mL) was added NH₄OH (5 mL) at room temperature. The reaction mixture was heated at 100° C. in an autoclave for 16 h. The reaction mixture was cooled and the solids formed were filtered to obtain 5-bromo-7-(2,2-difluorocyclopropyl)-7H-pyrrolo[2,3-d]pyrimidin-4-amine (0.06 g, 65%) as an pale yellow solid. LCMS (ES) m/z=289.0, 291.0 [M+H.]+. ¹H NMR (400 MHz, DMSO-d₆) δ ppm−2.24-2.43 (m, 2H), 4.19-4.26 (m, 1H), 6.77 (br.s, 2H), 7.45 (s, 1H), 8.12 (s, 1H).

Step 6: To a stirred solution of 3-(3,5-difluorobenzyl)-8-fluoro-7-(4,4,5,5-tetramethyl-1,3,2-dioxaborolan-2-yl)isoquinoline (0.075 g, 0.23 mmol, 1 equiv) in 1,4-Dioxane (30 mL) was added 5-bromo-7-(2,2-difluorocyclopropyl)-7H-pyrrolo[2,3-d]pyrimidin-4-amine (0.055 g, 0.18 mmol, 0.8 equiv), Tripotassium phosphate (0.1 g, 0.47 mmol, 2.0 equiv) and water (0.2 mL). The reaction mixture was degassed with N₂ for 15 minutes. Pd₂(dba)₃ (0.01 g, 0.011 mmol, 0.05 equiv) and (tBut)₃HPBF₄ (0.006 g, 0.023 mmol, 0.1 equiv) were added and degassed with N₂ for further 5 min. The reaction mixture was stirred for 10 h at 100° C. in a sealed vessel. The reaction was cooled to room temperature. The Reaction mixture was evaporated to obtain crude product. The crude product was purified over silica gel flash column chromatography. The compound eluted out in 3% MeOH:DCM to give 5-(3-(3,5-difluorobenzyl)-8-fluoroisoquinolin-7-yl)-7-(2,2-difluorocyclopropyl)-7H-pyrrolo[2,3-d]pyrimidin-4-amine (0.03 g, 26%) as an off white solid. LCMS (ES) m/z=482.1 [M+H]⁺. ¹H NMR (400 MHz, DMSO-d₆) δ ppm −2.30-2.38 (m, 2H), 4.29-4.37 (m, 3H), 6.26 (br.s, 2H), 7.04-7.06 (m, 3H), 7.46 (s, 1H), 7.72 (t, J=8.0 Hz, 1H), 7.80-7.84 (m, 2H), 8.18 (s, 1H), 9.42 (s, 1H).

### Example 12

### 3-(3-(3,5-difluorobenzyl)-8-fluoroisoquinolin-7-yl)-7-fluoro-1-methyl-1H-pyrrolo[3,2-c]pyridin-4-amine

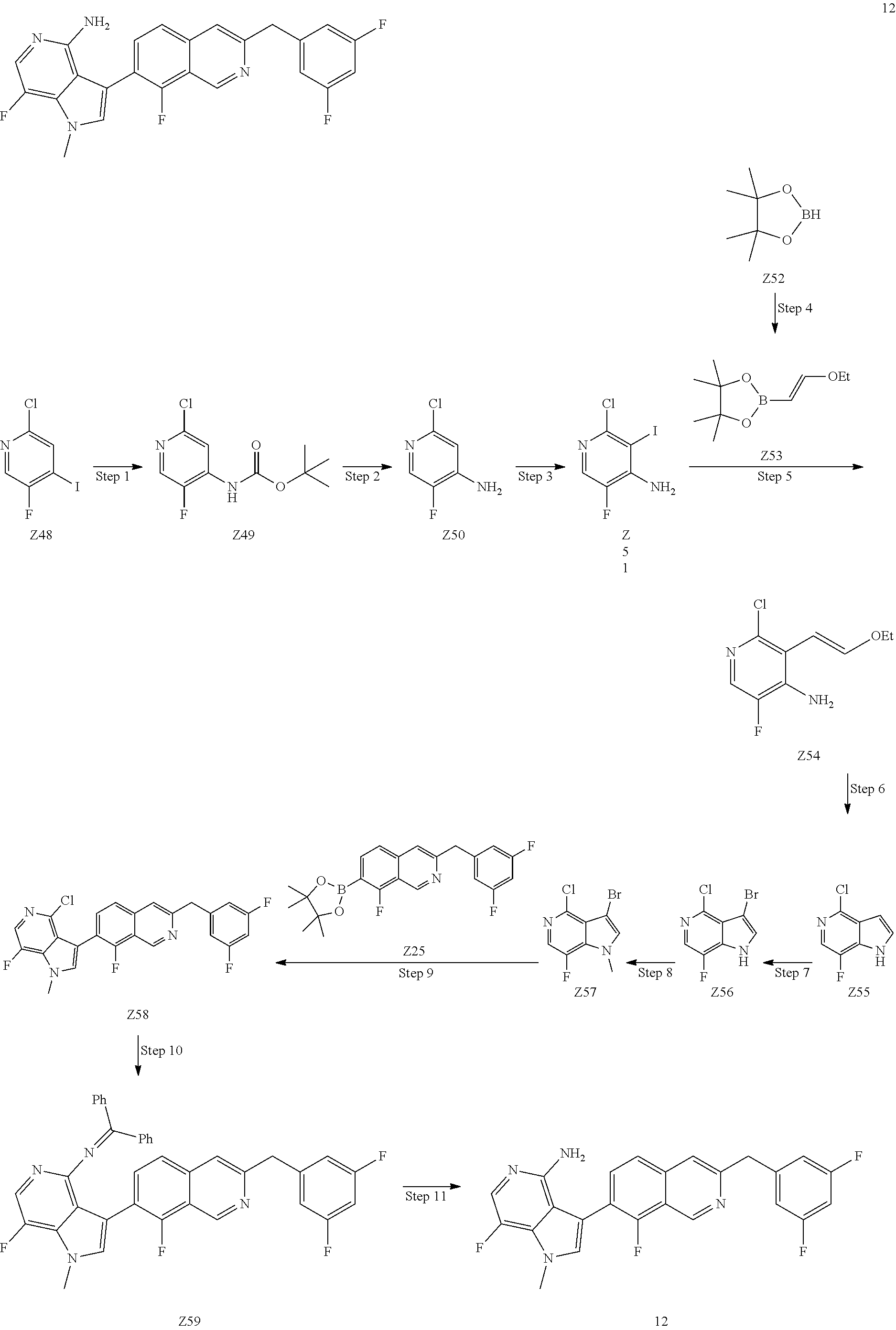

Step 1: A stirred solution of 2-chloro-5-fluoro-4-iodopyridine (5 g, 19.42 mmol, 1 equiv), tert-butyl carbamate (2.39 g, 20.4 mmol, 1.05 eq) and Cesium carbonate (12.66 g, 38.85 mmol, 2 equiv) in toluene (120 ml) was degassed with N₂ for 10 min. Pd₂(dba)₃ (0.36 g, 0.39 mmol, 0.02 equiv) and Xantphos (0.34 g, 0.58 mmol, 0.03 equiv) were added and the reaction mixture was stirred for 16 h at 100° C. After the consumption of starting material, the reaction mixture was cooled to room temperature and filtered through celite bed and washed with EtOAc (100 mL). The filtrate was washed with water, brine solution and concentrated to give the crude product. The crude product was purified by silica gel flash column chromatography. The compound eluted out in 40% EtOAc:Hexane. The pure fractions were evaporated to obtain tert-butyl (2-chloro-5-fluoropyridin-4-yl)carbamate as pale yellow solid (3.6 g, 75%). LCMS (ES) m/z=247.1 [M+H]⁺. ¹H NMR (400 MHz, DMSO-d₆) δ ppm 1.47 (s, 9H), 7.98 (d, J=5.6 Hz, 1H), 8.28 (d, J=2.8 Hz, 1H), 9.95 (s, 1H).

Step 2: A solution of tert-butyl (2-chloro-5-fluoropyridin-4-yl)carbamate (3.5 g, 14.2 mmol) in 60% TFA/DCM (25 ml) was stirred at room temperature for 1 h. After consumption of the starting material, the reaction mixture was concentrated under vacuum to give the crude product. The crude product was basified with saturated sodium bicarbonate solution and extracted with EtOAc (2×100 ml). The organic layer was dried over sodium sulphate and evaporated under vacuum to obtain 2-chloro-5-fluoropyridin-4-amine as pale yellow solid (1.9 g, 91.4%). LCMS (ES) m/z=147.1 [M+H]⁺. ¹H NMR (400 MHz, DMSO-d₆) δ ppm 6.54 (s, 2H), 6.65 (d, J=6.0 Hz, 1H), 7.89 (d, J=2.8 Hz, 1H).

Step 3: To a stirred solution of 2-chloro-5-fluoropyridin-4-amine (1.9 g, 12.97 mmol, 1 equiv) and sodium acetate (2.13 g, 25.94 mmol, 2 equiv) in acetic acid (20 ml) was added ICI (2.1 g, 12.97 mmol, 1 equiv) in acetic acid (5 ml) and stirred at 70° C. for 3 hours. After consumption of the starting material, the reaction mixture was poured into ice-cooled water and extracted with EtOAc (2×100 ml). The organic layer was washed with saturated sodium bicarbonate solution and 10% sodium thiosulphate solution. The organic layer was dried over sodium sulphate and evaporated under vacuum to obtain crude product. The crude product was purified by silica gel flash column chromatography. The compound eluted out in 40% EtOAc:Hexane. The pure fractions were evaporated to obtain 2-chloro-5-fluoro-3-iodopyridin-4-amine as light brown solid (2.5 g, 70.6%). LCMS (ES) m/z=272.9 [M+H]⁺. ¹H NMR (400 MHz, DMSO-d₆) δ ppm 6.63 (s, 2H), 7.93 (d, J=2.0 Hz, 1H).

Step 4: To a stirred solution of ethoxyethyne (2.5 g, 35.7 mmol, 1 equiv) in DCM (60 ml) at 0° C. was added 4,4,5,5-tetramethyl-1,3,2-dioxaborolane (5.69 ml, 39.2 mmol, 1.1 equiv) and Bis(cyclopentadienyl)Zirconium (IV) chloride hydride (0.55 g, 2.14 mmol, 0.06 eq). The reaction mixture was stirred at room temperature for 12 h. After consumption of the starting material, the reaction mixture was filtered through a pad of neutral alumina topped with a layer of celite and washed with DCM (50 ml). The filtrate obtained was evaporated under vacuum to obtain crude (E)-2-(2-ethoxyvinyl)-4,4,5,5-tetramethyl-1,3,2-dioxaborolane as a brown liquid (6.5 g). ¹H NMR (400 MHz, CDCl₃) δ ppm 1.25 (s, 12H), 1.25-1.29 (m, 3H), 3.81-3.91 (m, 2H), 4.43 (d, J=14.4 Hz, 1H), 7.03 (d, J=14.8 Hz, 1H).

Step 5: A stirred solution of 2-chloro-5-fluoro-3-iodopyridin-4-amine (2 g, 7.34 mmol, 1 equiv), (E)-2-(2-ethoxyvinyl)-4,4,5,5-tetramethyl-1,3,2-dioxaborolane (2.91 g, 14.62 mmol, 2 equiv), Potassium phosphate (3.1 g, 14. 62 mmol, 2 equiv) in acetonitrile:water (3:2, 30 ml:20 ml) was degassed with N₂ for 10 minutes. Palladium acetate (49. 4 mg, 0.22 mmol, 0.03 equiv) and ‘S’ phos (226 mg, 0.55 mmol, 0.075 equiv) were added and the reaction mixture was stirred for 16 h at 110° C. After consumption of the starting material, the reaction mixture was cooled to room temperature and filtered through celite bed and washed with EtOAc (100 mL). The filtrate was washed with water, brine solution and concentrated to give the crude product. The crude product was purified by silica gel flash column chromatography. The compound eluted out in 60% EtOAc:Hexane. The pure fractions were evaporated to obtain (E)-2-chloro-3-(2-ethoxyvinyl)-5-fluoropyridin-4-amine as pale yellow solid (1.4 g, 88%). LCMS (ES) m/z=217.1 [M+H]⁺. ¹H NMR (400 MHz, DMSO-d₆) δ ppm 1.25 (t, J=6.8 Hz, 3H), 3.94 (q, J=6.8 Hz, 2H), 5.42 (d, J=13.2 Hz, 1H), 6.23 (s, 2H), 6.82 (d, J=12.8 Hz, 1H), 7.80 (d, J=2.4 Hz, 1H).

Step 6: A stirred solution of (E)-2-chloro-3-(2-ethoxyvinyl)-5-fluoropyridin-4-amine (1.4 g, 6.46 mmol) in Ethanol (22 ml) and concentrated. HCl (5 ml) was stirred at 90° C. for 2 h. After consumption of the starting material, the reaction mixture was cooled to room temperature and basified with aqueous sodium bicarbonate solution. The aqueous solution was extracted with EtOAc (5×100 mL). The organic layer was dried over sodium sulphate and evaporated under vacuum to obtain crude product. The crude product was purified by silica gel flash column chromatography. The compound eluted out in 40% EtOAc:Hexane. The pure fractions were evaporated to obtain 4-chloro-7-fluoro-1H-pyrrolo[3,2-c]pyridine as pale yellow solid (0.9 g, 81.1%). LCMS (ES) m/z=170.9 [M+H]⁺. ¹H NMR (400 MHz, DMSO-d₆) δ ppm 6.62 (d, J=2.0 Hz, 1H), 7.64 (s, 1H), 7.98 (d, J=2.0 Hz, 1H), 12.55 (s, 1H).

Step 7: To a stirred solution of 4-chloro-7-fluoro-1H-pyrrolo[3,2-c]pyridine (0.4 g, 2.34 mmol, 1 equiv) in DMF (10 ml) was added NBS (0.42 g, 2.34 mmol, 1 equiv) and stirred at room temperature for 3 h. After consumption of the starting material, the reaction mixture was diluted with ethylacetate (50 ml), washed with water (2×50 ml) and brine solution. The organic layer was dried over sodium sulphate and evaporated under vacuum to obtain 3-bromo-4-chloro-7-fluoro-1H-pyrrolo[3,2-c]pyridine as pale yellow solid (550 mg, 94.2%). LCMS (ES) m/z=249.0, 250.0 [M+H]⁺.

Step 8: To a stirred solution of 3-bromo-4-chloro-7-fluoro-1H-pyrrolo[3,2-c]pyridine (540 mg, 2.16 mmol, 1 equiv) in DMF (15 ml) at 0° C. was sodium hydride (103.8 mg, 2.60 mmol, 1.2 equiv) and stirred for 10 minutes. Methyl iodide (0.2 ml, 3.25 mmol, 1.5 equiv) was added and stirred at room temperature for 2 hours. After consumption of the starting material, the reaction mixture was quenched with water and extracted with EtOAc (2×25 ml). The organic layer was dried over sodium sulphate and evaporated under vacuum to obtain the crude product. The crude product was purified by silica gel flash column chromatography. The compound eluted out in 50% EtOAc:Hexane. The pure fractions were evaporated to obtain 3-bromo-4-chloro-7-fluoro-1-methyl-1H-pyrrolo[3,2-c]pyridine as pale yellow solid (400 mg, 70.3%). LCMS (ES) m/z=262.9, 264.9 [M+H]⁺. ¹H NMR (400 MHz, DMSO-d₆) δ ppm 3.95 (s, 3H), 7.82 (s, 1H), 8.04 (d, J=3.2 Hz, 1H).

Step 9: A stirred solution of 3-bromo-4-chloro-7-fluoro-1-methyl-1H-pyrrolo[3,2-c]pyridine (160 mg, 0.61 mmol, 1 equiv), 3-(3,5-difluorobenzyl)-8-fluoro-7-(4,4,5,5-tetramethyl-1,3,2-dioxaborolan-2-yl)isoquinoline (266.6 mg, 0.67 mmol, 1.1 eq) and potassium phosphate (257.4 mg, 1.21 mmol, 2 equiv) in Dioxane:water (21 ml:7 ml) was degassed with N₂ for 10 minutes. Pd₂(dba)₃ (27.8 mg, 0.03 mmol, 0.05 equiv) and P(t-Bu)₃HBF₄ (17.6 mg, 0.06 mmol, 0.1 equiv) were added and the reaction mixture was stirred for 2 hour at 110° C. After consumption of the starting material, the reaction mixture was diluted with ethylacetate (25 ml) and washed with water and brine solution. The organic layer was dried over sodium sulphate and evaporated under vacuum to get the crude product. The crude product was purified by silica gel flash column chromatography. The compound eluted out in 70% EtOAc:Hexane. The pure fractions were evaporated to obtain 7-(4-chloro-7-fluoro-1-methyl-1H-pyrrolo[3,2-c]pyridin-3-yl)-3-(3,5-difluorobenzyl)-8-fluoroisoquinoline as pale yellow solid (240 mg, 86%). LCMS (ES) m/z=456.1 [M+H]⁺. ¹H NMR (400 MHz, DMSO-d₆) δ ppm 4.07 (s, 3H), 4.30 (s, 2H), 7.00-7.12 (m, 3H), 7.74-7.84 (m, 3H), 7.86 (s, 1H), 8.07 (d, J=3.2 Hz, 1H), 9.44 (s, 1H).

Step 10: A stirred solution of 7-(4-chloro-7-fluoro-1-methyl-1H-pyrrolo[3,2-c]pyridin-3-yl)-3-(3,5-difluorobenzyl)-8-fluoroisoquinoline (220 mg, 0.48 mmol, 1 equiv), tertdiphenylmethanimine (0.1 ml, 0.58 mmol, 1.2 eq) and sodium tert-butoxide (92.8 mg, 0.96 mmol, 2 equiv) in toluene (20 ml) was degassed with N₂ for 10 min. Pd₂(dba)₃ (22.1 mg, 0.024 mmol, 0.05 equiv) and BINAP (45.1 mg, 0.072 mmol, 0.15 equiv) were added and the reaction mixture was stirred for 16 hour at 110° C. After consumption of the starting material, the reaction mixture was diluted with ethylacetate (25 ml), washed with water and brine solution. The organic layer was dried over sodium sulphate and concentrated to get the crude product. The crude product was washed with pentane and dried under vacuum to get crude N-(3-(3-(3,5-difluorobenzyl)-8-fluoroisoquinolin-7-yl)-7-fluoro-1-methyl-1H-pyrrolo[3,2-c]pyridin-4-yl)-1,1-diphenylmethanimine (400 mg). (LCMS (ES) m/z=601.2 [M+H]⁺.

Step 11: To a stirred solution of N-(3-(3-(3,5-difluorobenzyl)-8-fluoroisoquinolin-7-yl)-7-fluoro-1-methyl-1H-pyrrolo[3,2-c]pyridin-4-yl)-1,1-diphenylmethanimine (400 mg, 0.66 mmol, 1 equiv) in Methanol (25 ml) was added aqueous solution of NH₂OH.HCl (462.9 mg, 6.66 mmol, 10 equiv) and aqueous solution of sodium bicarbonate (559.4 mg, 6.66 mmol, 10 equiv). The reaction mixture was stirred for 3 hour at room temperature. After consumption of the starting material, the reaction mixture was concentrated under vacuum to get the crude product. The crude product was dissolved in ethylacetate (50 ml), washed with water and brine solution. The organic layer was dried over sodium sulphate and evaporated under vacuum to give the crude product. The crude product was purified by silica gel flash column chromatography. The product eluted out in 3% MeOH:DCM. The pure fractions were evaporated to obtain 3-(3-(3,5-difluorobenzyl)-8-fluoroisoquinolin-7-yl)-7-fluoro-1-methyl-1H-pyrrolo[3,2-c]pyridin-4-amine as pale yellow solid (45 mg, 15.5%). LCMS (ES) m/z=437.1 [M+H]⁺. ¹H NMR (400 MHz, DMSO-d₆) δ ppm 3.96 (s, 3H), 4.30 (s, 2H), 5.04 (s, 2H), 7.02-7.08 (m, 3H), 7.44 (s, 1H), 7.57 (d, J=4.0 Hz, 1H), 7.72 (t, J=7.6 Hz, 1H), 7.81 (d, J=8.4 Hz, 1H), 7.85 (s, 1H), 9.43 (s, 1H).

### Example 13

### 5-(3-(3,5-difluorobenzyl)-8-fluoroisoquinolin-7-yl)-7-(oxetan-3-yl)-7H-pyrrolo[2,3-d]pyrimidin-4-amine

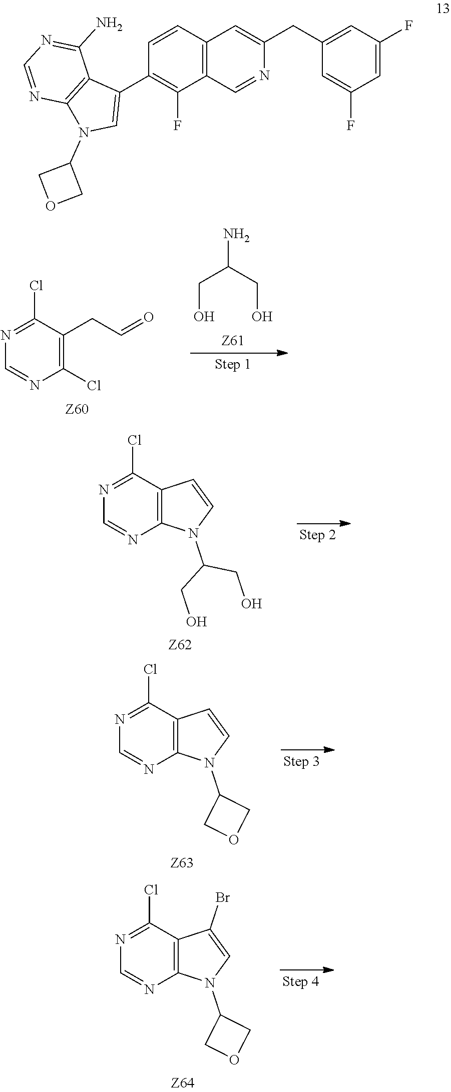 [SMILES: Nc1ncnc2c1c(-c1ccc3cc(Cc4cc(F)cc(F)c4)ncc3c1F)cn2C1COC1 ; O=CCc1c(Cl)ncnc1Cl ; NC(CO)CO ; OCC(CO)n1ccc2c(Cl)ncnc21 ; Clc1ncnc2c1ccn2C1COC1 ; Clc1ncnc2c1c(Br)cn2C1COC1]

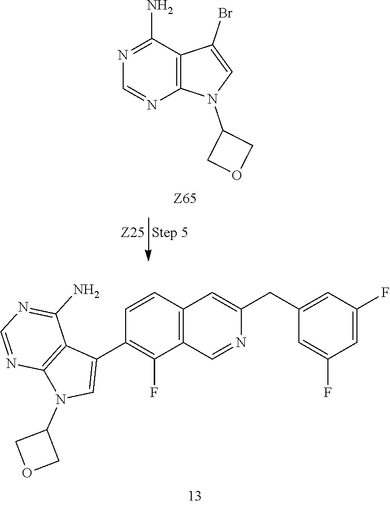 [SMILES: Nc1ncnc2c1c(Br)cn2C1COC1 ; Nc1ncnc2c1c(-c1ccc3cc(Cc4cc(F)cc(F)c4)ncc3c1F)cn2C1COC1]

Step 1: A stirred solution of 2-(4,6-dichloropyrimidin-5-yl)acetaldehyde (1) (3.1 g, 16.2 mmol, 1.0 equiv) and 2-aminopropane-1,3-diol (2) (3.69 g, 37.3 mmol, 2.3 equiv) in EtOH (60 mL) was refluxed for 2 h. After completion of starting material, reaction mixtures was concentrated and the residue was dissolved in DCM (150 mL). DCM layer was washed with water and brine solution, dried over Na₂SO₄, filtered and concentrated to give crude product. Crude product was purified by flash chromatography on silica gel and compound was eluted with 5% MeOH in DCM to give 2-(4-chloro-7H-pyrrolo[2,3-d]pyrimidin-7-yl)propane-1,3-diol (2.29 g, 59.7%) as off white solid. LC-MS (ES) m/z=228.1 [M+H]⁺. ¹H NMR (400 MHz, DMSO-d6) δ ppm 3.05-3.01 (m, 1H), 3.27-3.20 (m, 1H), 3.66-3.55 (m, 3H), 4.05-3.94 (m, 2H), 5.00 (t, J=5.6 Hz, 1H), 5.20 (d, J=6.4 Hz, 1H), 8.46 (s, 1H).

Step 2: To a stirred solution of 2-(4-chloro-7H-pyrrolo[2,3-d]pyrimidin-7-yl)propane-1,3-diol (3) (2.23 g, 9.69 mmol, 1.0 equiv) in THF (50 mL) was added nBuLi (1.2 M in THF) (8.8 mL, 10.6 mmol, 1.1 equiv) at −78° C. and the mixture was stirred for 2 h at that temperature, then added a solution of pTsCl (2.03 g, 10.6 mmol, 1.1 equiv) in THF (15 mL) at −78° C. and the mixture was slowly allowed to warm to 0° C. and stirred for 2 h at 0° C. After that again nBuLi (1.2 M in THF) (8.8 mL, 10.6 mmol, 1.1 equiv) was added at 0° C. and stirred at 60° C. for overnight. The reaction mixture was cooled to room temperature and quenched with Saturated NH₄Cl solution and extracted with EtOAc (3×150 mL). The combined organic layer was washed with water and brine solution, dried over Na₂SO₄, filtered and concentrated to give crude product. Crude product was purified by flash chromatography on silicagel and compound was eluted with 20% EtOAc/Hexane. Fractions containing pure compound was concentrated to give 4-chloro-7-(oxetan-3-yl)-7H-pyrrolo[2,3-d]pyrimidine (0.26 g, 13%) as white solid. LC-MS (ES) m/z=210.1 [M+H]⁺. ¹H NMR (400 MHz, DMSO-d6) δ ppm 5.02-4.97 (m, 4H), 5.96-5.89 (m, 1H), 6.75 (d, J=3.6 Hz, 1H), 8.13 (d, J=4.0 Hz, 1H), 8.63 (s, 1H).

Step 3: To a stirred solution of 4-chloro-7-(oxetan-3-yl)-7H-pyrrolo[2,3-d]pyrimidine (4) (0.1 g, 0.37 mmol, 1.0 equiv) in DCM (5 mL) was added NBS (0.072 g, 0.4 mmol, 1.1 equiv) at 0° C. and the mixture was stirred for 2 h at room temperature. After consumption of starting material, the reaction mixture was diluted with DCM (50 mL) and washed with water, saturated NaHCO₃ solution and brine solution. The organic layer was dried over Na₂SO₄, filtered and concentrated to give crude 5-bromo-4-chloro-7-(oxetan-3-yl)-7H-pyrrolo[2,3-d]pyrimidine (0.1 g, crude) as off white solid. LC-MS (ES) m/z=287.9, 289.0 [M+H]⁺. ¹H NMR (400 MHz, DMSO-d6) δ ppm 5.00-4.93 (m, 4H), 5.95-5.88 (m, 1H), 8.41 (s, 1H), 8.66 (s, 1H).

Step 4: 5-bromo-4-chloro-7-(oxetan-3-yl)-7H-pyrrolo[2,3-d]pyrimidine (0.25 g, 0.86 mmol, 1.0 equiv) and aqueous NH₃ (10 mL) in 1,4-Dioxane (10 mL) were taken in a steal bomb and heated to 100° C., stirred for 15 h. After consumption of starting material the reaction mixture was concentrated to give 5-bromo-7-(oxetan-3-yl)-7H-pyrrolo[2,3-d]pyrimidin-4-amine as off white solid (0.24 g, crude). LC-MS (ES) m/z=269.0, 271.0 [M+H]⁺.

Step 5: A stirred solution of 3-(3,5-difluorobenzyl)-8-fluoro-7-(4,4,5,5-tetramethyl-1,3,2-dioxaborolan-2-yl)Isoquinoline (0.42 g, 1.07 mmol, 1.2 equiv), 5-bromo-7-(oxetan-3-yl)-7H-pyrrolo[2,3-d]pyrimidin-4-amine (0.24 g, 0.89 mmol, 1.0 equiv) and potassium phosphate (0.37 g, 0.17 mmol, 2.0 equiv) in 1,4-Dioxane:water (4 mL: 1 mL) (20 mL) was degassed with N₂ for 15 minutes then Pd₂(dba)₃ (0.041 g, 0.044 mmol, 0.05 equiv), Tri-tert-butylphosphonium tetrafluoroborate (0.025 g, 0.089 mmol, 0.1 equiv) were added, and the reaction mixture was further degassed for 5 minutes. The reaction mixture was heated to 100° C. for 3 h. The reaction mixture was filtered through celite and filtrate was dried over Na₂SO₄, filtered and concentrated to obtain crude compound. Crude product was purified by flash column chromatography using silicagel column and compound was eluted at 2% MeOH:DCM. Fractions containing pure compound was concentrated to give 5-(3-(3,5-difluorobenzyl)-8-fluoroisoquinolin-7-yl)-7-(oxetan-3-yl)-7H-pyrrolo[2,3-d]pyrimidin-4-amine (0.185 g, 38%) as off white solid. LCMS (ES) m/z=462.1 [M+H]⁺. ¹H NMR (400 MHz, DMSO-d6) δ ppm 4.30 (s, 2H), 5.03-4.96 (m, 4H), 5.91-5.84 (m, 1H), 6.24 (bs, 2H), 7.05 (d, J=8.0 Hz, 3H), 7.85-7.75 (m, 4H), 8.13 (s, 1H), 9.43 (s, 1H). HPLC: 99.33% purity at 254 nM.

### Example 14

### 7-cyclopropyl-5-(3-(3,5-difluorobenzyl)-8-fluoro-4-methylisoquinolin-7-yl)-7H-pyrrolo[2,3-d]pyrimidin-4-amine

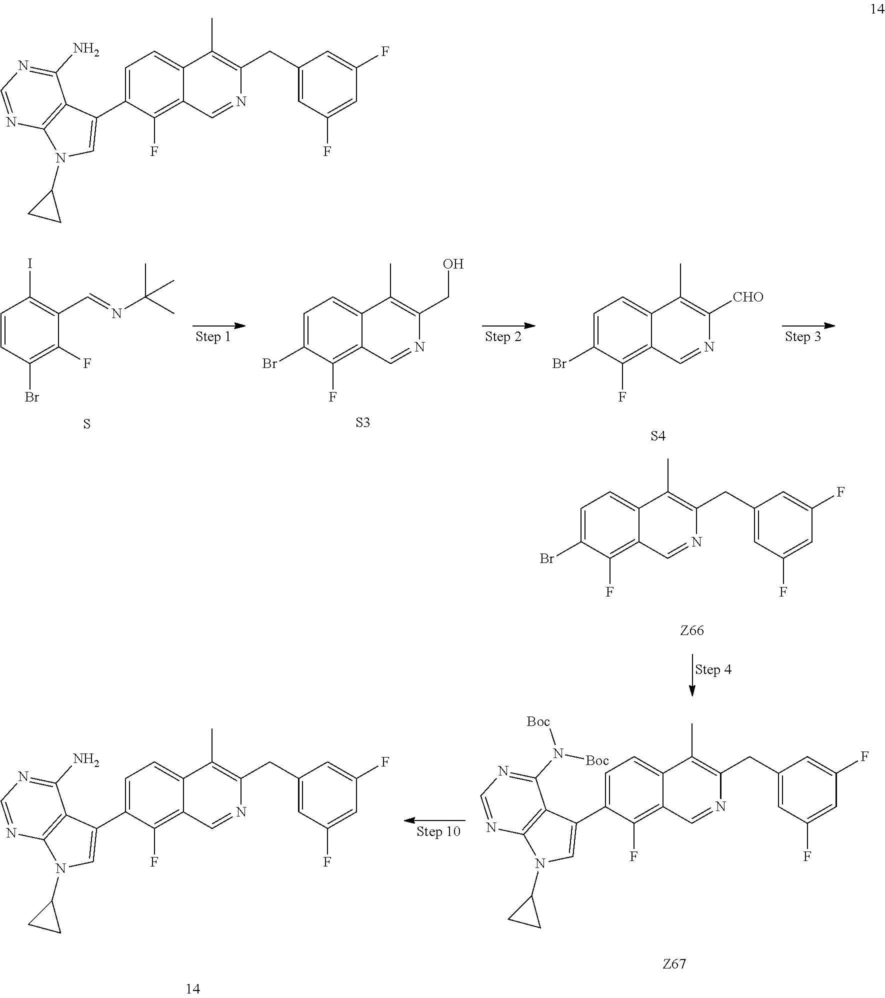

Step 1: To a stirred solution of 1-(3-bromo-2-fluoro-6-iodophenyl)-N-(tert-butyl)methanimine and but-2-yn-1-ol in DMF was added Na₂CO₃ and Pd(PPh₃)₄ under N₂ atmosphere, then heated to 100° C. for 3 h. After 3 h, reaction mixture was diluted with water and extracted with EtOAc (3×100 mL). Combined organic layer was washed with water and brine solution, dried over Na₂SO₄, filtered and concentrated to give crude product. Crude product was purified by flash column chromatography using silicagel column and compound was eluted at 40% EtOAc/Hexane. Fractions containing compound was concentrated to obtain (7-bromo-8-fluoro-4-methylisoquinolin-3-yl)methanol (0.9 g, 12.9%) as pale yellow solid. LCMS (ES) m/z=269.0, 271.0 [M+H]⁺. ¹H NMR (400 MHz, DMSO-d6) δ ppm 4.77 (d, J=5.6 Hz, 2H), 5.20 (t, J=5.6 Hz, 1H), 7.99-7.90 (m, 2H), 9.28 (s, 1H).

Step 2: To a stirred solution of (7-bromo-8-fluoro-4-methylisoquinolin-3-yl)methanol (0.8 g, 2.98 mmol, 1.0 equiv) in DCM (30 mL) was added Dess-Martin periodinane (2.53 g, 5.97 mmol, 2.0 equiv) at 0° C. and stirred for 3 h. After consumption of the starting material, the reaction mixture was poured onto a 1:1 mixture solution of sat NaHCO₃ and Na₂S₂O₃ solution (200 mL) and stirred for 30 min. The organic layer was separated washed with water and brine solution, dried over Na₂SO₄, filtered and concentrated to give crude product. Crude product was purified by flash column chromatography using silica gel column and compound was eluted at 20% EtOAc/Hexane. Fractions containing product was concentrated to give 7-bromo-8-fluoro-4-methylisoquinoline-3-carbaldehyde as off-white solid (0.41 g, 52%) LCMS (ES) m/z=267.0, 269.0 [M+H]⁺. ¹H NMR (400 MHz, DMSO-d6) δ ppm 2.95 (s, 3H), 8.18-8.13 (m, 2H), 9.50 (s, 1H), 10.33 (s, 1H).

Step 3: A stirred solution of 7-bromo-8-fluoro-4-methylisoquinoline-3-carbaldehyde (0.4 g, 1.5 mmol, 1.0 equiv) and Tosylhydrazine (0.3 g, 1.64 mmol, 1.1 equiv) in 1,4-Dioxane (30 mL) was heated to 80° C. and stirred for 2 h. After consumption of the starting material, (3,5-difluorophenyl)boronic acid (0.7 g, 4.47 mmol, 3.0 equiv) and K₃PO₄ (0.63 g, 3.0 mmol, 2.0 equiv) were added and the mixture was heated to 110° C. for 4 h. The reaction mixture was diluted with EtOAc (100 mL) and washed with water, saturated NaHCO₃ solution and brine solution, dried over Na₂SO₄, filtered and concentrated to give crude product. Crude product was purified by flash chromatography on silica gel and compound was eluted with 10% EtOAc/Hexane. Fractions containing product was concentrated to give 7-bromo-3-(3,5-difluorobenzyl)-8-fluoro-4-methylisoquinoline (0.25 g, 45%) as off white solid. LC-MS (ES) m/z=366.0, 368.0 [M+H]⁺. ¹H NMR (400 MHz, CDCl₃) δ ppm 2.58 (s, 3H), 4.38 (s, 2H), 6.62 (t, J=9.2 Hz, 1H), 6.71 (d, J=6.4 Hz, 2H), 6.66 (d, J=8.8 Hz, 1H), 7.78 (t, J=7.2 Hz, 1H), 9.39 (s, 1H).

Step 4: A stirred solution of N,N-Di(tert-butoxycarbonyl)7-cyclopropyl-5-(4,4,5,5-tetramethyl-1,3,2-dioxaborolan-2-yl)-7H-pyrrolo[2,3-d]pyrimidin-4-amine (0.19 g, 0.52 mmol, 1.0 equiv), 7-bromo-3-(3,5-difluorobenzyl)-8-fluoro-4-methylisoquinoline (0.31 g, 0.62 mmol, 1.2 equiv) and potassium phosphate (0.264 g, 1.24 mmol, 2.0 equiv) in 1,4-Dioxane:water (6 mL: 2 mL) was degassed with N₂ for 15 minutes. Pd₂(dba)₃ (0.028 g, 0.031 mmol, 0.05 equiv), and Tri-tert-butylphosphonium tetrafluoroborate (0.018 g, 0.062 mmol, 0.1 equiv) were added and the reaction mixture was further degassed for 5 minutes. The reaction mixture was heated to 100° C. for 2 h. The reaction mixture was filtered through celite and filtrate was dried over Na₂SO₄, filtered and concentrated to obtain crude compound. Crude product was purified by flash column chromatography using silicagel column. Product was eluted at 30% EtOAc/Hexane. Fractions containing product were concentrated to give N,N-Di(tert-butoxycarbonyl)7-cyclopropyl-5-(3-(3,5-difluorobenzyl)-8-fluoro-4-methylisoquinolin-7-yl)-7H-pyrrolo[2,3-d]pyrimidin-4-amine (0.22 g, 55%) as off white solid. LCMS (ES) m/z=660.3 [M+H]⁺. ¹H NMR (400 MHz, DMSO-d6) δ ppm 1.05 (bs, 22H), 2.60 (s, 3H), 3.78-3.74 (m, 1H), 4.43 (s, 2H), 6.87 (d, J=7.2 Hz, 2H), 7.02 (t, J=9.2 Hz, 1H), 7.76 (t, J=8.0 Hz, 1H), 7.91 (d, J=8.8 Hz, 1H), 7.96 (s, 1H), 8.82 (s, 1H), 9.28 (s, 1H).

Step 5: To a stirred solution of N,N-Di(tert-butoxycarbonyl)7-cyclopropyl-5-(3-(3,5-difluorobenzyl)-8-fluoro-4-methylisoquinolin-7-yl)-7H-pyrrolo[2,3-d]pyrimidin-4-amine (0.22 g, 0.33 mmol, 1.0 equiv) in DCM (10 mL) was added 4M HCl in dioxane (3 mL) at 0° C. and stirred for 5 h at room temperature. After completion of starting material, reaction mixture was concentrated under reduced pressure and adjusted pH-8 using sat NaHCO₃ solution. The obtained solid was filtered and washed with diethyl ether and dried to give 7-cyclopropyl-5-(3-(3,5-difluorobenzyl)-8-fluoro-4-methylisoquinolin-7-yl)-7H-pyrrolo[2,3-d]pyrimidin-4-amine as off-white solid (0.11 g, 72%). LCMS (ES) m/z=460.2 [M+H]⁺. ¹H NMR (400 MHz, DMSO-d6) δ ppm 1.05-1.02 (m, 4H), 2.61 (s, 3H), 3.62-3.58 (m, 1H), 4.04 (s, 2H), 6.18 (br.s., 2H), 6.92 (d, J=7.2 Hz, 2H), 7.04-6.98 (m, 1H), 7.37 (s, 1H), 7.76 (t, J=8.4 Hz, 1H), 7.97 (d, J=8.4 Hz, 1H), 8.16 (s, 1H), 9.32 (s, 1H). HPLC: 99.46% purity at 254 nM.

The Compounds 15 to 60 were prepared generally according to the procedures described in the Schemes 1 to 6 and Examples 1 to 14.

TABLE 1

| Compound | | | LCMS | ¹H-NMR (400 MHz, |
|---|---|---|---|---|
| # | Structure | Name | (MH⁺¹) | DMSO-d₆) |
| 15 | 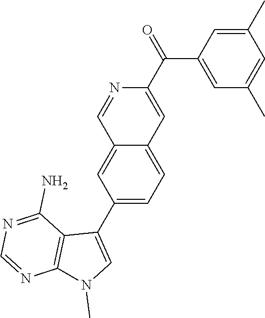 [SMILES: Cc1cc(C)cc(C(=O)c2cc3ccc(-c4cn(C)c5ncnc(N)c45)cc3cn2)c1] | (7-(4-amino-7- methyl-7H- pyrrolo[2,3- d]pyrimidin-5- yl)isoquinolin-3- yl)(3,5- dimethylphenyl) methanone | 408.2 | 2.35 (s, 6H), 3.79 (s, 3H), 6.26 (br.s., 2H), 7.30 (s, 1H), 7.57 (s, 3H), 8.00 (d, J = 8.4 Hz, 1H), 8.20-8.23 (m, 2H), 8.27 (d, J = 8.4 Hz, 1H), 8.51 (s, 1H). 9.42 (s, 1H). |
| 16 | 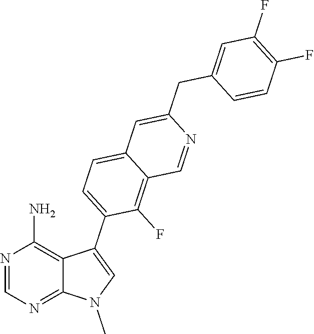 [SMILES: Cn1cc(-c2ccc3cc(Cc4ccc(F)c(F)c4)ncc3c2F)c2c(N)ncnc21] | 5-(3-(3,4- difluorobenzyl)- 8- fluoroisoquinolin- 7-yl)-7-methyl- 7H-pyrrolo[2,3- d]pyrimidin-4- amine | 420.1 | 3.75 (s, 3H), 4.26 (s, 2H), 6.13 (br. s., 2H), 7.11-7.18 (m, 1H), 7.30-7.41 (m, 3H), 7.71 (t, J = 8.0 Hz, 1H), 7.78-7.80 (m, 2H), 8.14 (s, 1H), 9.41 (s, 1H). |
| 17 | 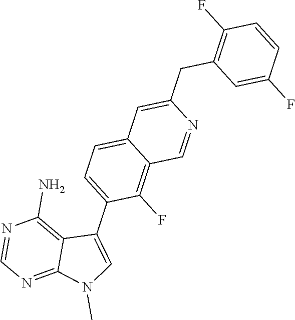 [SMILES: Cn1cc(-c2ccc3cc(Cc4cc(F)ccc4F)ncc3c2F)c2c(N)ncnc21] | 5-(3-(2,5- difluorobenzyl)- 8- fluoroisoquinolin- 7-yl)-7-methyl- 7H-pyrrolo[2,3- d]pyrimidin-4- amine | 420.1 | 3.76 (s, 3H), 4.29 (s, 2H), 6.14 (br. s., 2H), 7.08-7.18 (m, 1H), 7.20-7.23 (m, 2H), 7.42 (s, 1H), 7.71 (t, J = 8.0 Hz, 1H), 7.77 (s, 1H), 7.81 (d, J = 8.4 Hz, 1H), 8.15 (s, 1H), 9.41 (s, 1H). |
| 18 | 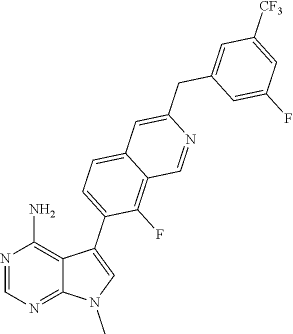 [SMILES: Cn1cc(-c2ccc3cc(Cc4cc(F)cc(C(F)(F)F)c4)ncc3c2F)c2c(N)ncnc21] | 5-(8-fluoro-3-(3- fluoro-5- (trifluorometh- yl)benzyl)iso- quinolin-7- yl)-7-methyl- 7H-pyrrolo[2,3- d]pyrimidin-4- amine | 470.1 | 3.75 (s, 3H), 4.39 (s, 2H), 6.14 (br. s., 2H), 7.41 (s, 1H), 7.51 (t, J = 8.4 Hz, 2H), 7.58 (s, 1H), 7.72 (t, J = 8.0 Hz, 1H), 7.81 (d, J = 8.4 Hz, 1H), 7.88 (s, 1H), 8.14 (s, 1H), 9.42 (s, 1H). |
| 19 | 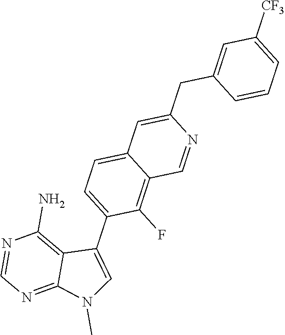 [SMILES: Cn1cc(-c2ccc3cc(Cc4cccc(C(F)(F)F)c4)ncc3c2F)c2c(N)ncnc21] | 5-(8-fluoro-3-(3- (trifluorometh- yl)benzyl)iso- quinolin-7- yl)-7-methyl- 7H-pyrrolo[2,3- d]pyrimidin-4- amine | 452.1 | 3.75 (s, 3H), 4.37 (s, 2H), 6.14 (br. s., 2H), 7.41 (s, 1H), 7.50-7.57 (m, 2H), 7.64 (d, J = 7.2 Hz, 1H), 7.69-7.72 (m, 2H), 7.80 (d, J = 8.4 Hz, 1H), 7.86 (s, 1H), 8.14 (s, 1H), 9.41 (s, 1H). |
| 20 | 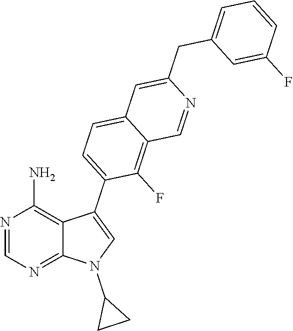 [SMILES: Nc1ncnc2c1c(-c1ccc3cc(Cc4cccc(F)c4)ncc3c1F)cn2C1CC1] | 7-cyclopropyl-5- (8-fluoro-3-(3- fluorobenzyl)iso- quinolin-7-yl)- 7H-pyrrolo[2,3- d]pyrimidin-4- amine | 428.1 | 0.98-1.08 (m, 4H), 3.59 (s, 1H), 4.28 (s, 2H), 6.12 (br. s., 2H), 7.01 (t, J = 7.6 Hz, 1H), 7.12-7.16 (m, 2H), 7.30-7.34 (m, 2H), 7.70 (t, J = 7.6 Hz, 1H), 7.71-7.81 (m, 2H), 8.15 (s, 1H), 9.40 (s, 1H). |
| 21 | 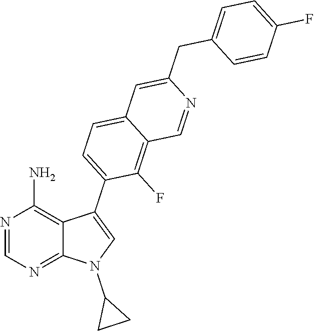 [SMILES: Nc1ncnc2c1c(-c1ccc3cc(Cc4ccc(F)cc4)ncc3c1F)cn2C1CC1] | 7-cyclopropyl-5- (8-fluoro-3-(4- fluorobenzyl)iso- quinolin-7-yl)- 7H-pyrrolo[2,3- d]pyrimidin-4- amine | 428.1 | 0.98-1.08 (m, 4H), 3.56-3.61 (m 1H), 4.24 (s, 2H), 6.12 (br. s., 2H), 7.10 (t, J = 8.8 Hz, 2H), 7.33-7.36 (m, 3H), 7.70 (t, J = 8.0 Hz, 1H), 7.76-7.78 (m, 2H), 8.15 (s, 1H), 9.39 (s, 1H). |
| 22 | 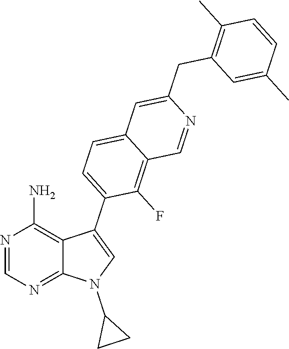 [SMILES: Cc1ccc(C)c(Cc2cc3ccc(-c4cn(C5CC5)c5ncnc(N)c45)c(F)c3cn2)c1] | 7-cyclopropyl-5- (3-(2,5- dimethylbenzyl)- 8- fluoroisoquinolin- 7-yl)-7H- pyrrolo[2,3- d]pyrimidin-4- amine | 438.2 | 0.99-1.07 (m, 4H), 2.22 (s, 3H), 2.23 (s, 3H), 3.54-3.62 (m, 1H), 4.22 (s, 2H), 6.12 (br. s., 2H), 6.94 (d, J = 7.6 Hz, 1H), 7.02-7.08 (m, 2H), 7.34 (s, 1H), 7.57 (s, 1H), 7.67 (t, J = 7.2 Hz, 1H), 7.75 (d, J = 8.8 Hz, 1H), 8.15 (s, 1H), 9.40 (s, 1H). |
| 23 | 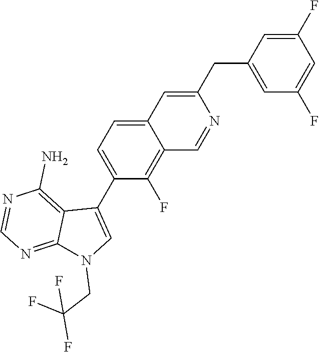 [SMILES: Nc1ncnc2c1c(-c1ccc3cc(Cc4cc(F)cc(F)c4)ncc3c1F)cn2CC(F)(F)F] | 5-(3-(3,5- difluorobenzyl)- 8- fluoroisoquinolin- 7-yl)-7-(2,2,2- trifluoroethyl)- 7H-pyrrolo[2,3- d]pyrimidin-4- amine | 488.1 | 4.29 (s, 2H), 5.05-5.20 (m, 2H), 6.45 (br.s, 2H), 6.80-7.01 (m, 3H), 7.50 (s, 1H), 7.73 (t, J = 7.2 Hz, 1 H), 7.82 (d, J = 8.4 Hz, 1 H), 7.86 (s, 1 H), 8.22 (s, 1 H), 9.43 (s, 1 H). |
| 24 | 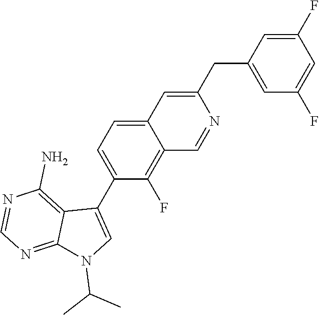 [SMILES: CC(C)n1cc(-c2ccc3cc(Cc4cc(F)cc(F)c4)ncc3c2F)c2c(N)ncnc21] | 5-(3-(3,5- difluorobenzyl)- 8- fluoroisoquinolin- 7-yl)-7-isopropyl- 7H-pyrrolo[2,3- d]pyrimidin-4- amine | 448.2 | 1.46 (d, J = 6.8 Hz, 6 H), 4.29 (s, 2H), 4.95-5.00 (m, 1H), 6.12 (br. S., 2H), 7.00-7.10 (m, 3H), 7.55 (s, 1H), 7.74 (t, J = 7.6 Hz, 1 H), 7.80 (d, J = 8.4 Hz, 1 H), 7.83 (s, 1 H), 8.12 (s, 1 H), 9.42 (s, 1 H). |
| 25 | 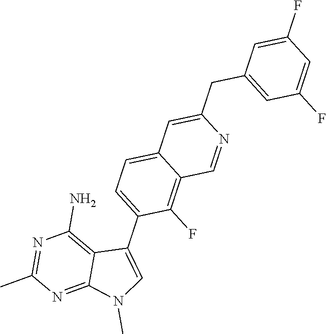 [SMILES: Cc1nc(N)c2c(-c3ccc4cc(Cc5cc(F)cc(F)c5)ncc4c3F)cn(C)c2n1] | 5-(3-(3,5- difluorobenzyl)- 8- fluoroisoquinolin- 7-yl)-2,7- dimethyl-7H- pyrrolo[2,3- d]pyrimidin-4- amine | 434.1 | 2.40 (s, 3 H), 3.71 (s, 3H), 4.28 (s, 2H), 6.05 (br. S., 2H), 7.03-7.05 (m, 3H), 7.32 (s, 1H), 7.70 (t, J = 7.6 Hz, 1 H), 7.79 (d, J = 8.4 Hz, 1 H), 7.83 (s, 1 H), 9.41 (s, 1 H). |
| 26 | 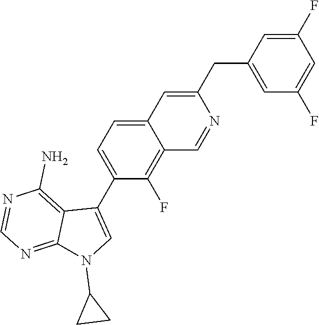 [SMILES: Nc1ncnc2c1c(-c1ccc3cc(Cc4cc(F)cc(F)c4)ncc3c1F)cn2C1CC1] | 7-cyclopropyl-5- (3-(3,5- difluorobenzyl)- 8- fluoroisoquinolin- 7-yl)-7H- pyrrolo[2,3- d]pyrimidin-4- amine | 446.1 | 1.02-1.05 (m, 4H), 3.55-3.65 (m, 1H), 4.29 (s, 2H), 6.13 (br. S., 2H), 7.00-7.06 (m, 3H), 7.35 (s, 1H), 7.72 (t, J = 7.6 Hz, 1 H), 7.78- 7.81 (m, 1H), 7.83 (s, 1H), 8.15 (s, 1 H), 9.41 (s, 1 H). |
| 27 | 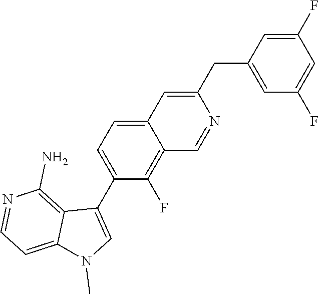 [SMILES: Cn1cc(-c2ccc3cc(Cc4cc(F)cc(F)c4)ncc3c2F)c2c(N)nccc21] | 3-(3-(3,5- difluorobenzyl)- 8- fluoroisoquinolin- 7-yl)-1-methyl- 1H-pyrrolo[3,2- c]pyridin-4-amine | 419.1 | 3.77 (s, 3H), 4.30 (s, 2H), 5.15 (s, 2H), 6.82 (d, J = 5.6 Hz, 1 H), 7.05- 7.07 (m, 3H), 7.38 (s, 1H), 7.68 (d, J = 6 Hz, 1 H), 7.74 (t, J = 7.6 Hz, 1 H), 7.80-7.82 (m, 1H), 7.85 (s, 1 H), 9.43 (s, 1 H). |
| 28 | 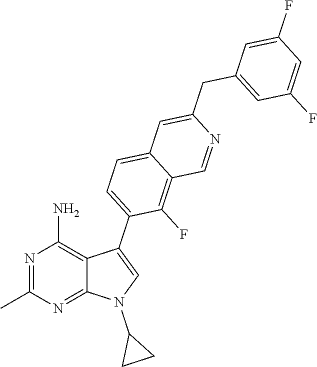 [SMILES: Cc1nc(N)c2c(-c3ccc4cc(Cc5cc(F)cc(F)c5)ncc4c3F)cn(C3CC3)c2n1] | 7-cyclopropyl-5- (3-(3,5- difluorobenzyl)- 8- fluoroisoquinolin- 7-yl)-2-methyl- 7H-pyrrolo[2,3- d]pyrimidin-4- amine | 460.1 | 0.98-1.05 (m, 4H), 2.41 (s, 3H), 3.55-3.59 (m, 1H), 4.28 (s, 2H), 6.03 (br. S., 2H), 6.98- 7.08 (m, 3H), 7.24 (s, 1H), 7.70 (t, J = 8 Hz, 1 H), 7.78 (d, J = 8.8 Hz, 1 H), 7.82 (s, 1H), 9.40 (s, 1H). |
| 29 | 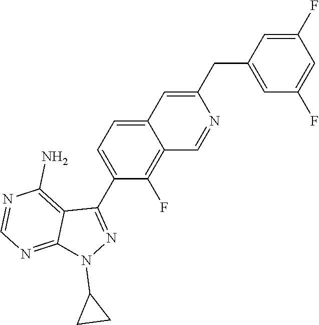 [SMILES: Nc1ncnc2c1c(-c1ccc3cc(Cc4cc(F)cc(F)c4)ncc3c1F)nn2C1CC1] | 1-cyclopropyl-3- (3-(3,5- difluorobenzyl)- 8- fluoroisoquinolin- 7-yl)-1H- pyrazolo[3,4- d]pyrimidin-4- amine | 447.1 | 1.07 (d, J = 5.6 Hz, 2 H), 1.20 (s, 2H), 3.85-3.93 (m, 1H), 4.30 (s, 2H), 7.00-7.10 (m, 3H), 6.25-7.50 (br. S., 2H), 7.83 (s, 2H), 7.88 (s, 1 H), 8.23 (s, 1 H), 9.44 (s, 1 H). |
| 30 | 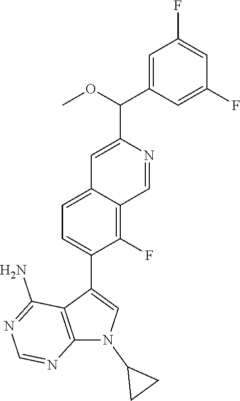 [SMILES: COC(c1cc(F)cc(F)c1)c1cc2ccc(-c3cn(C4CC4)c4ncnc(N)c34)c(F)c2cn1] | 7-cyclopropyl-5- (3-((3,5- difluorophen- yl)(meth- oxy)methyl)-8- fluoroisoquinolin- 7-yl)-7H- pyrrolo[2,3- d]pyrimidin-4- amine | 476.2 | 0.99-1.05 (m, 4H), 3.39 (s, 3H), 3.56-3.62 (m, 1H), 5.62 (s, 1H), 6.14 (br.s, 2H), 7.08- 7.14 (m, 3H), 7.34 (s, 1H), 7.75 (t, J = 8.0 Hz, 1H), 7.90 (d, J = 8.4 Hz, 1H), 8.07 (s, 1H), 8.15 (s, 1H), 9.39 (s, 1H) |
| 31 | 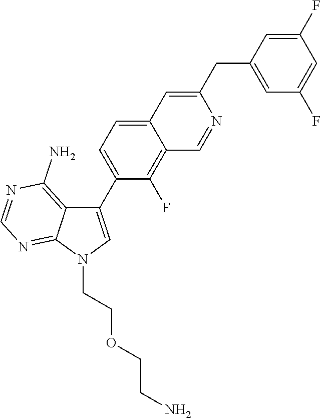 [SMILES: NCCOCCn1cc(-c2ccc3cc(Cc4cc(F)cc(F)c4)ncc3c2F)c2c(N)ncnc21] | 7-(2-(2- aminoeth- oxy)ethyl)-5- (3-(3,5- difluorobenzyl)- 8- fluoroisoquinolin- 7-yl)-7H- pyrrolo[2,3- d]pyrimidin-4- amine | 493.2 | 2.59-2.61 (m, 2H), 3.37 (t, J = 9.6 Hz, 2H), 3.76-3.78 (m, 2H), 4.29 (s, 2H), 4.34-4.35 (m, 2H), 6.15 (br.s., 2H), 7.04 (s, 3H), 7.46 (s, 1H), 7.72 (d, J = 7.6 Hz, 1H), 7.82 (t, J = 9.0 Hz, 2H), 8.14 (s, 1H). 9.42 (s, 1H). |
| 32 | 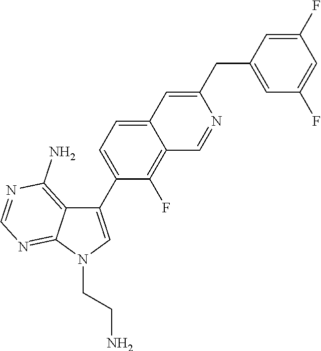 [SMILES: NCCn1cc(-c2ccc3cc(Cc4cc(F)cc(F)c4)ncc3c2F)c2c(N)ncnc21] | 7-(2-aminoethyl)- 5-(3-(3,5- difluorobenzyl)- 8- fluoroisoquinolin- 7-yl)-7H- pyrrolo[2,3- d]pyrimidin-4- amine | 449.2 | 1.63-1.75 (m, 2H), 2.92 (t, J = 6.4 Hz, 2H), 4.15 (t, J = 6.2 Hz, 2H), 4.29 (s, 2H), 6.13 (br.s., 2H), 7.04-7.06 (m, 3H), 7.46 (s, 1H), 7.73 (t, J = 7.8 Hz, 1H), 7.80 (d, J = 8.4 Hz, 1H), 7.84 (s, 1H), 8.13 (s, 1H), 9.42 (s, 1H). |
| 33 | 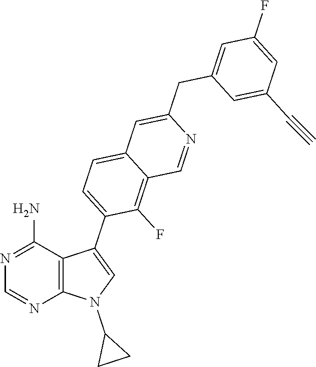 [SMILES: C#Cc1cc(F)cc(Cc2cc3ccc(-c4cn(C5CC5)c5ncnc(N)c45)c(F)c3cn2)c1] | 7-cyclopropyl-5- (3-(3-ethynyl-5- fluorobenzyl)-8- fluoroisoquinolin- 7-yl)-7H- pyrrolo[2,3- d]pyrimidin-4- amine | 452.2 | 0.80-0.90 (m, 4H), 3.59 (s, 1H), 4.25-4.27 (m, 3H), 6.12 (br. s., 2H), 7.17 (d, J = 8.8 Hz, 1H), 7.22 (s, 1H), 7.26 (d, J = 8.4 Hz, 1H), 7.34 (s, 1H), 7.71 (t, J = 7.8 Hz, 1H, 1H), 7.80 (d, J = 8.4 Hz, 1H), 7.84 (s, 1H), 8.15 (s, 1H), 9.41 (s, 1H) |
| 34 | 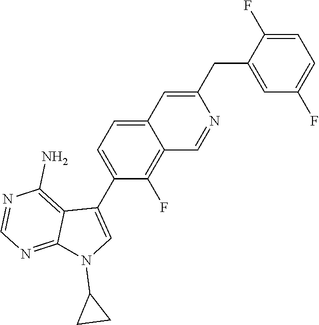 [SMILES: Nc1ncnc2c1c(-c1ccc3cc(Cc4cc(F)ccc4F)ncc3c1F)cn2C1CC1] | 7-cyclopropyl-5- (3-(2,5- difluorobenzyl)- 8- fluoroisoquinolin- 7-yl)-7H- pyrrolo[2,3- d]pyrimidin-4- amine | 446.1 | 1.02-1.05 (m, 4H), 3.56-3.62(m, 1H), 4.29 (s, 2H), 6.13 (br.S., 2H), 7.08-7.16 (m, 1H), 7.18-7.26 (m, 2H), 7.35 (s, 1 H), 7.69- 7.73 (m, 1H), 7.76- 7.81 (m, 2H), 8.15 (s, 1 H), 9.40 (s, 1 H). |
| 35 | 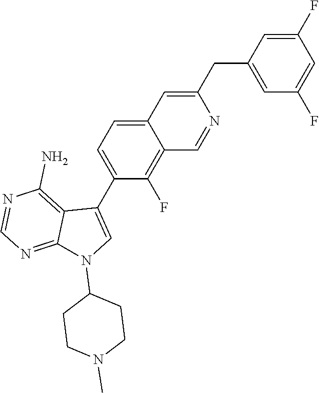 [SMILES: CN1CCC(n2cc(-c3ccc4cc(Cc5cc(F)cc(F)c5)ncc4c3F)c3c(N)ncnc32)CC1] | 5-(3-(3,5- difluorobenzyl)- 8- fluoroisoquinolin- 7-yl)-7-(1- methylpiperidin- 4-yl)-7H- pyrrolo[2,3- d]pyrimidin-4- amine | 503.2 | 1.85-1.95 (m, 2H), 2.20-2.12 (m, 4H), 2.21 (s, 3H), 2.89 (d, J = 4.8 Hz, 2H), 4.23 (s, 2H), 4.55 (br.S., 1H), 6.13 (br.S., 2H), 7.00- 7.10 (m, 3H), 7.54 (s, 1H), 7.73 (t, J = 7.6 Hz, 1 H), 7.80 (d, J = 8.8 Hz, 1 H), 7.83 (s, 1 H), 8.12 (s, 1 H), 9.42 (s, 1 H). |
| 36 | 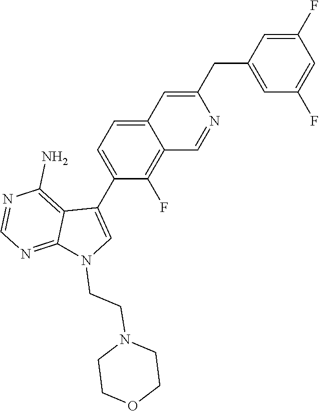 [SMILES: Nc1ncnc2c1c(-c1ccc3cc(Cc4cc(F)cc(F)c4)ncc3c1F)cn2CCN1CCOCC1] | 5-(3-(3,5- difluorobenzyl)- 8- fluoroisoquinolin- 7-yl)-7-(2- morpholinoethyl)- 7H-pyrrolo[2,3- d]pyrimidin-4- amine | 519.2 | 2.43 (br.S., 4H), 2.71 (t, J = 5.6 Hz, 2 H), 3.51 (br.S., 4H), 4.26- 4.34 (m, 4H), 6.14 (br.S., 2H), 7.02-7.10 (m, 3H), 7.49 (s, 1H), 7.72 (t, J = 7.2 Hz, 1H), 7.80 (d, J = 8.8 Hz, 1 H), 7.84 (s, 1H), 8.13 (s, 1H), 9.42 (s, 1 H). |
| 37 | 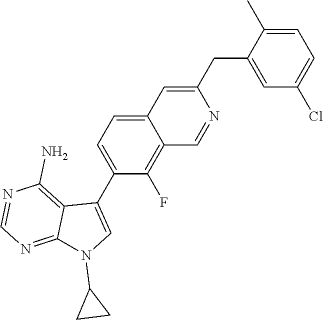 [SMILES: Cc1ccc(Cl)cc1Cc1cc2ccc(-c3cn(C4CC4)c4ncnc(N)c34)c(F)c2cn1] | 5-(3-(5-chloro-2- methylbenzyl)-8- fluoroisoquinolin- 7-yl)-7- cyclopropyl-7H- pyrrolo[2,3- d]pyrimidin-4- amine | 458.3 | 1.01-1.04 (m, 4H), 2.27 (s, 3H), 3.59 (br. S., 1H), 4.27 (s, 2H), 6.13 (br.S., 2H), 7.19 (s, 2H), 7.26 (s, 1H), 7.34 (s, 1H), 7.68-7.71 (m, 2H), 7.77-7.79 (m, 1H), 8.15 (s, 1H), 9.41 (s, 1 H). |
| 38 | 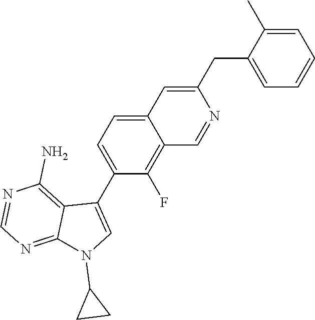 [SMILES: Cc1ccccc1Cc1cc2ccc(-c3cn(C4CC4)c4ncnc(N)c34)c(F)c2cn1] | 7-cyclopropyl-5- (8-fluoro-3-(2- methylbenzyl)iso- quinolin-7-yl)-7H- pyrrolo[2,3- d]pyrimidin-4- amine | 424.2 | 1.00-1.10 (m, 4H), 2.28 (s, 3H), 3.59 (br. S., 1H), 4.27 (s, 2H), 6.11 (br.S., 2H), 7.10-7.22 (m, 4H), 7.34 (s, 1H), 7.59 (s, 1H), 7.67 (t, J = 7.2 Hz, 1H), 7.73-7.75 (m, 1H), 8.14 (s, 1H), 9.40 (s, 1 H). |
| 39 | 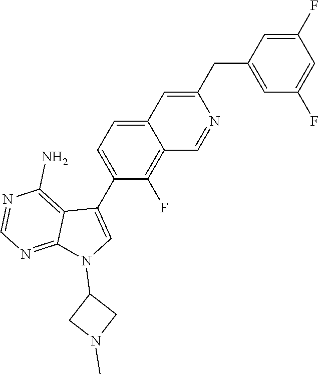 [SMILES: CN1CC(n2cc(-c3ccc4cc(Cc5cc(F)cc(F)c5)ncc4c3F)c3c(N)ncnc32)C1] | 5-(3-(3,5- difluorobenzyl)- 8- fluoroisoquinolin- 7-yl)-7-(1- methylazetidin-3- yl)-7H- pyrrolo[2,3- d]pyrimidin-4- amine | 475.2 | 2.34 (s, 3H), 3.40-3.45 (m, 2H), 3.71-3.75 (m, 2H), 4.29 (s, 2H), 5.26- 5.29 (m, 1H), 6.21 (br.S., 2H), 7.05-7.06 (m, 3H), 7.75-7.78 (m, 2H), 7.80-7.83 (m, 1 H), 7.85 (s, 1H), 8.12 (s, 1H), 9.43 (s, 1H). |
| 40 | 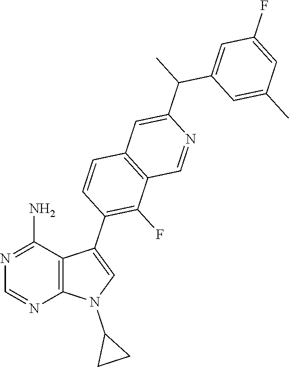 [SMILES: Cc1cc(F)cc(C(C)c2cc3ccc(-c4cn(C5CC5)c5ncnc(N)c45)c(F)c3cn2)c1] | 7-cyclopropyl-5- (3-(1-(3,5- difluorophen- yl)ethyl)-8- fluoroisoquinolin- 7-yl)-7H- pyrrolo[2,3- d]pyrimidin-4- amine | 460.2 | 1.05-1.02 (m, 4H), 1.70 (d, J = 7.2 Hz, 3H), 3.58 (t, J = 4.0 Hz, 1H), 4.51 (q, J = 6.8 Hz, 1H), 6.12 (bs, 2H), 7.01 (t, J = 9.2 Hz, 1H), 7.07 (d, J = 7.2 Hz, 2H), 7.34 (s, 1H), 7.71 (t, J = 7.6 Hz, 1H), 7.80 (d, J = 8.8 Hz, 1H), 7.86 (s, 1H), 8.14 (s, 1H), 9.42 (s, 1H). |
| 41 | 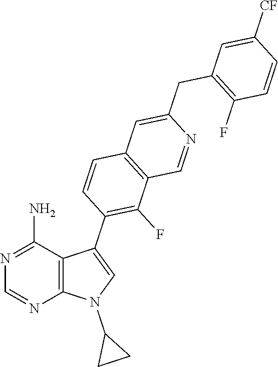 | 7-cyclopropyl-5- (8-fluoro-3-(2- fluoro-5- (trifluorometh- yl)benzyl)iso- quinolin-7- yl)-7H- pyrrolo[2,3- d]pyrimidin-4- amine | 496.1 | 1.04-1.01 (m, 4H), 3.59-3.58 (m, 1H), 4.39 (s, 2H), 6.13 (bs, 2H), 7.34 (s, 1H), 7.42 (t, J = 9.2 Hz, 1H), 7.73- 7.69 (m, 2H), 7.82- 7.81 (m, 3H), 8.14 (s, 1H), 9.39 (s, 1H). |
| 42 | 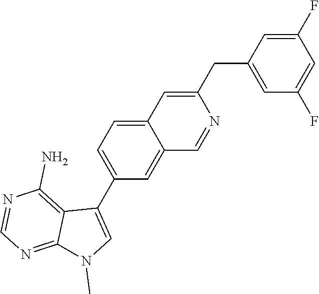 [SMILES: Cn1cc(-c2ccc3cc(Cc4cc(F)cc(F)c4)ncc3c2)c2c(N)ncnc21] | 5-(3-(3,5- difluoroben- zyl)isoquinolin- 7-yl)-7- methyl-7H- pyrrolo[2,3- d]pyrimidin-4- amine | 402.1 | 3.76 (s, 3H), 4.25 (s, 2H), 6.15 (br.S., 2H), 7.04 (d, J = 7.2 Hz, 3H), 7.45 (s, 1H), 7.75 (s, 1H), 7.84 (d, J = 8.4 Hz, 1H), 7.97 (d, J = 8.4 Hz, 1H), 8.07 (s, 1 H), 8.17 (s, 1 H), 9.26 (s, 1H). |
| 43 | 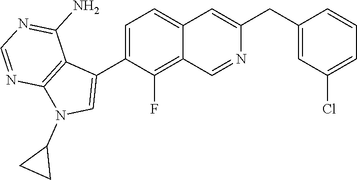 [SMILES: Nc1ncnc2c1c(-c1ccc3cc(Cc4cccc(Cl)c4)ncc3c1F)cn2C1CC1] | 5-(3-(3- chlorobenzyl)-8- fluoroisoquinolin- 7-yl)-7- cyclopropyl-7H- pyrrolo[2,3- d]pyrimidin-4- amine | 444.1 | 0.99-1.08 (m, 4H), 3.56- 3.62 (m, 1H), 4.27 (s, 2H), 6.12 (br.s., 2H), 7.24-7.34 (m, 4H), 7.38 (s, 1H), 7.71 (t, J = 7.2 Hz, 1H), 7.78 (d, J = 8.4 Hz, 1H), 7.82 (s, 1H), 8.15 (s, 1H), 9.40 (S, 1H). |
| 44 | 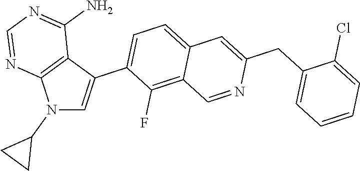 [SMILES: Nc1ncnc2c1c(-c1ccc3cc(Cc4ccccc4Cl)ncc3c1F)cn2C1CC1] | 5-(3-(2- chlorobenzyl)-8- fluoroisoquinolin- 7-yl)-7- cyclopropyl-7H- pyrrolo[2,3- d]pyrimidin-4- amine | 444.0 | 0.98-1.08 (m, 4H), 3.58-3.62 (m, 1H), 4.39 (s, 2H), 6.13 (br. s., 2H), 7.26-7.33 (m, 2H), 7.34 (s, 1H), 7.39 (d, J = 6.4 Hz, 1H), 7.45 (d, J = 7.2 Hz, 1H), 7.65 (s, 1H), 7.69 (t, J = 8.0 Hz, 1H), 7.77 (d, J = 8.8 Hz, 1H), 8.15 (s, 1H), 9.40 (s, 1H). |
| 45 | 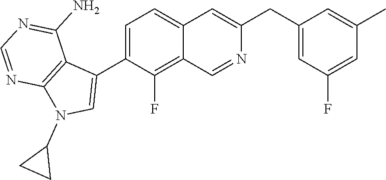 [SMILES: Cc1cc(F)cc(Cc2cc3ccc(-c4cn(C5CC5)c5ncnc(N)c45)c(F)c3cn2)c1] | 7-cyclopropyl-5- (8-fluoro-3-(3- fluoro-5- methylben- zyl)isoquinolin- 7-yl)-7H- pyrrolo[2,3- d]pyrimidin-4- amine | 442.1 | 0.96-1.08 (m, 4H), 2.26 (s, 3H), 3.56-3.62 (m, 1H), 4.22 (s, 2H), 6.12 (br. s., 2H), 6.84 (d, J = 9.6 Hz, 1H), 6.92 (d, J = 10.0 Hz, 1H), 6.97 (s, 1H), 7.34 (s, 1H), 7.70 (t, J = 7.6 Hz, 1H), 7.78 (d, J = 8.8 Hz, 2H), 8.15 (s, 1H), 9.40 (s, H). |
| 46 | 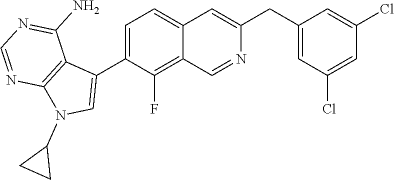 [SMILES: Nc1ncnc2c1c(-c1ccc3cc(Cc4cc(Cl)cc(Cl)c4)ncc3c1F)cn2C1CC1] | 7-cyclopropyl-5- (3-(3,5- dichlorobenzyl)- 8- fluoroisoquinolin- 7-yl)-7H- pyrrolo[2,3- d]pyrimidin-4- amine | 479.1 | 0.98-1.08 (m, 4H), 3.57-3.61 (m, 1H), 4.28 (s, 2H), 6.13 (br. s., 2H), 7.34 (s, 1H), 7.39 (s, 1H), 7.43 (s, 1H), 7.72 (t, J = 8.0 Hz, 1H), 7.80 (d, J = 8.4 Hz, 1H), 7.85 (s, 1H), 8.15 (s, 1H), 9.41 (s, 1H). |
| 47 | 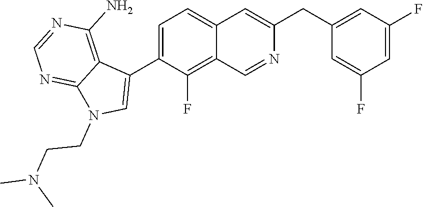 [SMILES: CN(C)CCn1cc(-c2ccc3cc(Cc4cc(F)cc(F)c4)ncc3c2F)c2c(N)ncnc21] | 5-(3-(3,5- difluorobenzyl)- 8- fluoroisoquinolin- 7-yl)-7-(2- (dimethyl- amino)ethyl)-7H- pyrrolo[2,3- d]pyrimidin-4- amine | 477.1 | 2.17 (s, 6H), 2.64-2.68 (m, 2H), 4.24-4.30 (m, 4H), 6.14 (br s., 2H), 7.00-7.08 (m, 3H), 7.48 (s, 1H), 7.70-7.74 (m, 1H), 7.79-7.84 (m, 2H), 8.13 (s, 1H), 9.42 (s, 1H). |
| 48 | 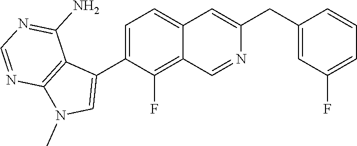 [SMILES: Cn1cc(-c2ccc3cc(Cc4cccc(F)c4)ncc3c2F)c2c(N)ncnc21] | 5-(8-fluoro-3-(3- fluorobenzyl)iso- quinolin-7-yl)-7- methyl-7H- pyrrolo[2,3- d]pyrimidin-4- amine | 402.1 | 3.75 (s, 3H), 4.28 (s, 2H), 6.13 (br. s., 2H), 6.98-7.04 (m, 1H), 7.12-7.16 (m, 2H), 7.29-7.35 (m, 1H), 7.41 (s, 1H), 7.68-7.72 (m, 1H), 7.78-7.81 (m, 2H), 8.14 (s, 1H), 9.41 (s, 1H). |
| 49 | 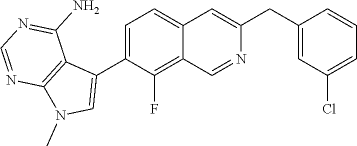 [SMILES: Cn1cc(-c2ccc3cc(Cc4cccc(Cl)c4)ncc3c2F)c2c(N)ncnc21] | 5-(3-(3- chlorobenzyl)-8- fluoroisoquinolin- 7-yl)-7-methyl- 7H-pyrrolo[2,3- d]pyrimidin-4- amine | 418.3 | 3.75 (s, 3H), 4.27 (s, 2H), 6.13 (br. s., 2H), 7.24-7.34 (m, 3H), 7.38 (s, 1H), 7.41 (s, 1H), 7.70 (t, J = 7.6 Hz, 1H), 7.78-7.83 (m, 2H), 8.14 (s, 1H), 9.41 (s, 1H). |
| 50 | 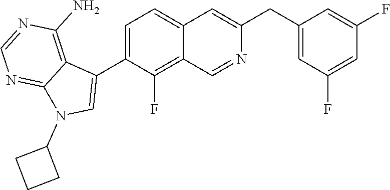 [SMILES: Nc1ncnc2c1c(-c1ccc3cc(Cc4cc(F)cc(F)c4)ncc3c1F)cn2C1CCC1] | 7-cyclobutyl-5- (3-(3,5- difluorobenzyl)- 8- fluoroisoquinolin- 7-yl)-7H- pyrrolo[2,3- d]pyrimidin-4- amine | 460.2 | 1.78-1.86 (m, 2H), 2.55-2.57 (m, 4H), 4.29 (s, 2H), 5.20 (t, J = 8.0 Hz, 1H), 6.14 (br. s., 2H), 7.04-7.06 (m, 3H), 7.69 (s, 1H), 7.75 (t, J = 8.0 Hz, 1H), 7.81 (d, J = 8.4 Hz, 1H), 7.84 (s, 1H), 8.12 (s, 1H), 9.42 (s, 1H). |
| 51 | 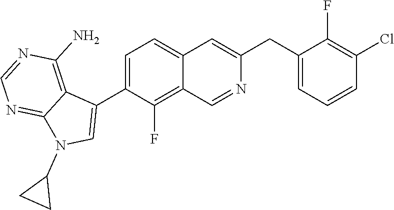 [SMILES: Nc1ncnc2c1c(-c1ccc3cc(Cc4cccc(Cl)c4F)ncc3c1F)cn2C1CC1] | 5-(3-(3-chloro-2- fluorobenzyl)-8- fluoroisoquinolin- 7-yl)-7- cyclopropyl-7H- pyrrolo[2,3- d]pyrimidin-4- amine | 462.1 | 0.99-1.08 (m, 4H), 3.56-3.62 (m, 1H), 4.34 (s, 2H), 6.13 (br. s., 2H), 7.17 (t, J = 8.0 Hz, 1H), 7.33-7.36 (m, 2H), 7.45 (t, J = 7.6 Hz, 1H), 7.71 (t, J = 8.0 Hz, 1H), 7.78-7.81 (m, 2H), 8.15 (s, 1H), 9.38 (s, 1H). |
| 52 | 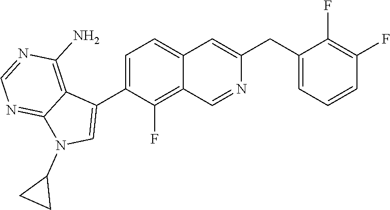 [SMILES: Nc1ncnc2c1c(-c1ccc3cc(Cc4cccc(F)c4F)ncc3c1F)cn2C1CC1] | 7-cyclopropyl-5- (3-(2,3- difluorobenzyl)- 8- fluoroisoquinolin- 7-yl)-7H- pyrrolo[2,3- d]pyrimidin-4- amine | 446.3 | 1.01-1.04 (m, 4H), 3.58- 3.59 (m, 1H), 4.34 (s, 2H), 6.13 (br.s., 2H), 7.14-7.18 (m, 2H), 7.25-7.32 (m, 1H), 7.35 (s, 1H), 7.69-7.73 (m, 1H), 7.77-7.81 (m, 2H), 8.14 (s, 1H), 9.39 (s, 1H). |
| 53 | 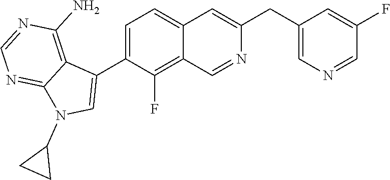 [SMILES: Nc1ncnc2c1c(-c1ccc3cc(Cc4cncc(F)c4)ncc3c1F)cn2C1CC1] | 7-cyclopropyl-5- (8-fluoro-3-((5- fluoropyridin-3- yl)methyl)iso- quinolin-7-yl)- 7H-pyrrolo[2,3- d]pyrimidin-4- amine | 429.2 | 1.01-1.05 (m, 4H), 3.58-3.59 (m, 1H), 4.34 (s, 2H), 6.12 (br. s., 2H), 7.35 (s, 1H), 7.65- 7.74 (m, 2H), 7.78- 7.80 (m, 1H), 7.86 (s, 1H), 8.15 (s, 1H), 8.41- 8.42 (m, 1H), 8.46 (s, 1H), 9.41 (s, 1H). |
| 54 | 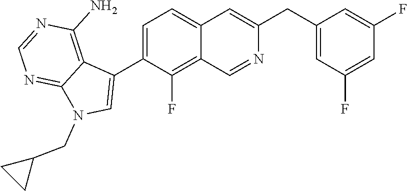 [SMILES: Nc1ncnc2c1c(-c1ccc3cc(Cc4cc(F)cc(F)c4)ncc3c1F)cn2CC1CC1] | 7-(cyclopropyl- methyl)-5-(3- (3,5- difluorobenzyl)- 8- fluoroisoquinolin- 7-yl)-7H- pyrrolo[2,3- d]pyrimidin-4- amine | 460.1 | 0.41-0.46 (m, 2H), 0.47-0.50 (m, 2H), 1.25-1.28 (m, 1H), 4.05 (d, J = 7.2 Hz, 2H), 4.29 (s, 2H), 6.14 (br. s., 2H), 7.02-7.06 (m, 3H), 7.52 (s, 1H), 7.74 (t, J = 8.4 Hz, 1H), 7.81 (d, J = 8.4 Hz, 1H), 7.84 (s, 1H), 8.13 (s, 1H), 9.43 (s, 1H). |
| 55 | 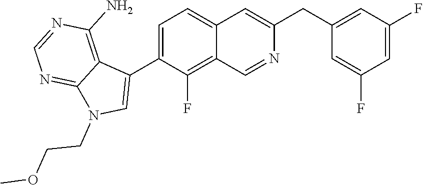 [SMILES: COCCn1cc(-c2ccc3cc(Cc4cc(F)cc(F)c4)ncc3c2F)c2c(N)ncnc21] | 5-(3-(3,5- difluorobenzyl)- 8- fluoroisoquinolin- 7-yl)-7-(2- methoxyethyl)- 7H-pyrrolo[2,3- d]pyrimidin-4- amine | 464.2 | 3.24 (s, 3H), 3.70-3.72 (m, 2H), 4.29 (s, 2H), 4.33-4.36 (m, 2H), 6.15 (br. s., 2H), 7.04- 7.06 (m, 3H), 7.44 (s, 1H), 7.72 (t, J = 8.0 Hz, 1H), 7.80 (d, J = 8.4 Hz, 1H), 7.84 (s, 1H), 8.13 (s, 1H), 9.42 (s, 1H). |
| 56 | 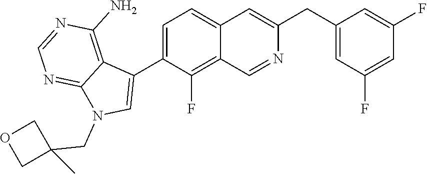 [SMILES: CC1(Cn2cc(-c3ccc4cc(Cc5cc(F)cc(F)c5)ncc4c3F)c3c(N)ncnc32)COC1] | 5-(3-(3,5- difluorobenzyl)- 8- fluoroisoquinolin- 7-yl)-7-((3- methyloxetan-3- yl)methyl)-7H- pyrrolo[2,3- d]pyrimidin-4- amine | 490.2 | 1.21 (s, 3H), 4.22 (d, J = 6.0 Hz, 2H), 4.29 (s, 2H), 4.41 (s, 2H), 4.65 (d, J = 6.0 Hz, 2H), 6.20 (br. s., 2H), 7.04 (d, J = 7.2 Hz, 3H), 7.47 (s, 1H), 7.73 (t, J = 8.0 Hz, 1H), 7.81 (d, J = 8.8 Hz, 1H), 7.84 (s, 1H), 8.15 (s, 1H), 9.42 (s, 1H). |
| 57 | 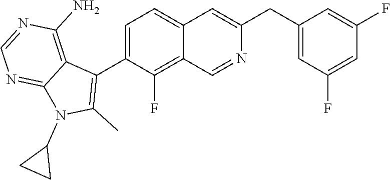 [SMILES: Cc1c(-c2ccc3cc(Cc4cc(F)cc(F)c4)ncc3c2F)c2c(N)ncnc2n1C1CC1] | 7-cyclopropyl-5- (3-(3,5- difluorobenzyl)- 8- fluoroisoquinolin- 7-yl)-6-methyl- 7H-pyrrolo[2,3- d]pyrimidin-4- amine | 460.2 | 1.13 (d, J = 4.8 Hz, 4H), 2.29 (s, 3H), 3.17-3.19 (m, 1H), 4.30 (s, 2H), 5.77 (br. s., 2H), 7.02- 7.06 (m, 3H), 7.63 (t, J = 8.0 Hz, 1H), 7.81 (d, J = 8.4 Hz, 1H), 7.86 (s, 1H), 8.08 (s, 1H), 9.42 (s, 1H). |
| 58 | 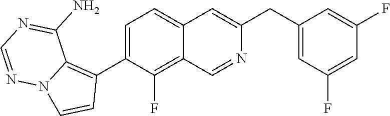 [SMILES: Nc1ncnn2ccc(-c3ccc4cc(Cc5cc(F)cc(F)c5)ncc4c3F)c12] | 5-(3-(3,5- difluorobenzyl)- 8- fluoroisoquinolin- 7-yl)pyrrolo[2,1- f][1,2,4]triazin-4- amine | 406.1 | 4.29 (s, 2H), 5.86-6.68 (br. s., 2H), 6.77 (d, J = 2.4 Hz, 1H), 7.02-7.05 (m, 3H), 7.71 (t, J = 8.0 Hz, 1H), 7.78-7.81 (m, 2H), 7.86 (d, J = 9.2 Hz, 2H), 9.42 (s, 1H). |
| 59 | 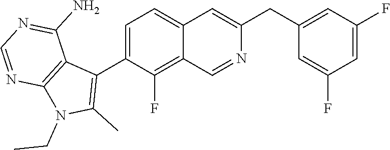 [SMILES: CCn1c(C)c(-c2ccc3cc(Cc4cc(F)cc(F)c4)ncc3c2F)c2c(N)ncnc21] | 5-(3-(3,5- difluorobenzyl)- 8- fluoroisoquinolin- 7-yl)-7-ethyl-6- methyl-7H- pyrrolo[2,3- d]pyrimidin-4- amine | 448.2 | 1.29 (t, J = 6.8 Hz, 3H), 2.25 (s, 3H), 4.20-4.26 (m, 2H), 4.30 (s, 2H), 5.80 (br. s., 2H), 7.01- 7.06 (m, 3H), 7.64 (t, J = 8.0 Hz, 1H), 7.81 (d, J = 8.4 Hz, 1H), 7.86 (s, 1H), 8.09 (s, 1H), 9.43 (s, 1H). |
| 60 | 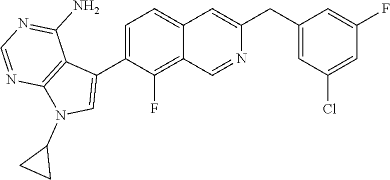 [SMILES: Nc1ncnc2c1c(-c1ccc3cc(Cc4cc(F)cc(Cl)c4)ncc3c1F)cn2C1CC1] | 5-(3-(3-chloro-5- fluorobenzyl)-8- fluoroisoquinolin- 7-yl)-7- cyclopropyl-7H- pyrrolo[2,3- d]pyrimidin-4- amine | 462.1 | 1.04 (s, 4H), 3.58-3.62 (m, 1H), 4.28 (s, 2H), 6.13 (br. s., 2H), 7.18 (d, J = 8.8 Hz, 1H), 7.22- 7.28 (m, 2H), 7.35 (s, 1H), 7.72 (t, J = 8.0 Hz, 1H), 7.79 (d, J = 8.8 Hz, 1H), 7.84 (s, 1H), 8.15 (s, 1H), 9.41 (s, 1H). |

### Example 61: PERK Enzyme Assay

Compounds of the invention were assayed for PERK enzyme inhibitory activity with modifications to previously reported conditions (Axten et al. J. Med. Chem., 2012, 55, 7193-7207). Briefly, various concentrations of compounds (maximum 1% DMSO) were dispensed into 384-well plates containing GST-PERK enzyme. ATP and biotin-elF2α were added and after 60 minutes the reaction was quenched. After 2 hrs, a fluorescence plate reader was used to measure inhibition and pIC50s were calculated. The activity of compound 1 was determined at an ATP concentration=5 μM. For compounds 2-20 the PERK activity was determined at ATP concentration=500 μM ATP.

### Example 62—Capsule Composition

An oral dosage form for administering the present invention is produced by filing a standard two piece hard gelatin capsule with the ingredients in the proportions shown in Table 2, below.

TABLE 2

| INGREDIENTS | AMOUNTS |
|---|---|
| 5-(3-Benzylisoquinolin-7-yl)-7-methyl-7H-pyrrolo[2,3- | 7 mg |
| d]pyrimidin-4-amine (Compound of Example 1) | |
| Lactose | 53 mg |
| Talc | 16 mg |
| Magnesium Stearate | 4 mg |

### Example 63—Injectable Parenteral Composition

An injectable form for administering the present invention is produced by stirring 1.7% by weight of 5-(3-(3,5-Dimethylbenzyl)isoquinolin-7-yl)-7-methyl-7H-pyrrolo[2,3-d]pyrimidin-4-amine (Compound of Example 2) in 10% by volume propylene glycol in water.

### Example 64 Tablet Composition

The sucrose, calcium sulfate dihydrate and a PERK inhibitor as shown in Table 3 below, are mixed and granulated in the proportions shown with a 10% gelatin solution. The wet granules are screened, dried, mixed with the starch, talc and stearic acid, screened and compressed into a tablet.

TABLE 3

| INGREDIENTS | AMOUNTS |
|---|---|
| 5-(3-Benzyl-8-fluoroisoquinolin-7-yl)-7-methyl-7H- | 12 mg |
| pyrrolo[2,3-d]pyrimidin-4-amine (Compound of Example 3) | |
| calcium sulfate dihydrate | 30 mg |
| sucrose | 4 mg |
| Starch | 2 mg |
| Talc | 1 mg |
| stearic acid | 0.5 mg |

### Biological Activity

Compounds of the invention are tested for activity against PERK in the above assay.

The compounds of Examples 2 to 60 were tested generally according to the above PERK enzyme assay and in at least one experimental run exhibited an average PERK Enzyme (500 μM ATP) pIC50 value: >5.4 against PERK, except for Examples 15, 18, 19, 23, 25, 29, and 58 which exhibited pIC50<5.4

The compound of Example 51 was tested generally according to the above PERK enzyme assay and in at least one set of experimental runs exhibited an average PERK Enzyme (500 μM ATP) pIC50 value of 8.5 against PERK.

The compound of Example 53 was tested generally according to the above PERK enzyme assay and in at least one set of experimental runs exhibited an average PERK Enzyme (500 μM ATP) pIC50 value of 6.2 against PERK.

The compound of Example 47 was tested generally according to the above PERK enzyme assay and in at least one set of experimental runs exhibited an average PERK Enzyme (500 μM ATP) pIC50 value of 5.8 against PERK.

The compound of Example 41 was tested generally according to the above PERK enzyme assay and in at least one set of experimental runs exhibited an average PERK Enzyme (500 μM ATP) pIC50 value of 7.0 against PERK.

The compound of Example 6 was tested generally according to the above PERK enzyme assay and in at least one set of experimental runs exhibited an average PERK Enzyme (500 μM ATP) pIC50 value of 6.8 against PERK.

The compound of Example 28 was tested generally according to the above PERK enzyme assay and in at least one set of experimental runs exhibited an average PERK Enzyme (500 μM ATP) pIC50 value of 5.6 against PERK.

The compound of Example 17 was tested generally according to the above PERK enzyme assay and in at least one set of experimental runs exhibited an average PERK Enzyme (500 μM ATP) pIC50 value of 6.0 against PERK.

The compound of Example 38 was tested generally according to the above PERK enzyme assay and in at least one set of experimental runs exhibited an average PERK Enzyme (500 μM ATP) pIC50 value of 7.9 against PERK.

The compound of Example 4 was tested generally according to the above PERK enzyme assay and in at least one set of experimental runs exhibited an average PERK Enzyme (500 μM ATP) pIC50 value of 6.6 against PERK.

The compound of Example 1 was tested generally according to the above PERK enzyme assay and in at least one set of experimental runs exhibited an average PERK Enzyme (5 μM ATP) pIC50 value of 7.8 against PERK. (Note: the compound of Example 1 was tested at 5 μM ATP).

While the preferred embodiments of the invention are illustrated by the above, it is to be understood that the invention is not limited to the precise instructions herein disclosed and that the right to all modifications coming within the scope of the following claims is reserved.

1. A method of treating a disease selected from: cancer, pre-cancerous syndromes, spinal cord injury, traumatic brain injury, ischemic stroke, stroke, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, cognitive impairment, atherosclerosis, ocular diseases, arrhythmias, in organ transplantation and in the transportation of organs for transplantation, in a mammal in need thereof, which comprises administering to such mammal a therapeutically effective amount of a compound of Formula I: 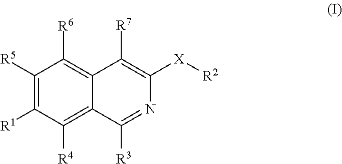
wherein:
R¹ is selected from:
bicycloheteroaryl,
substituted bicycloheteroaryl,
heteroaryl, and
substituted heteroaryl,
where said substituted bicycloheteroaryl and said substituted heteroaryl are substituted with from one to five substituents independently selected from:
fluoro,
chloro,
bromo,
iodo,
C₁₋₆alkyl,
C₁₋₆alkyl substituted with from 1 to 5 substituents independently selected from: fluoro, chloro, bromo, iodo, C₁₋₄alkyloxy, —OH, C₁₋₄alkyl, cycloalkyl, —COOH, —CF₃, —NO₂, —NH₂ and —CN,
—OH,
hydroxyC₁₋₆alkyl,
—COOH,
tetrazole,
cycloalkyl,
oxo,
—OC₁₋₆alkyl,
—CF₃,
—CF₂H,
—CFH₂,
C₁₋₆alkylOC₁₋₄alkyl,
—CONH₂,
—CON(H)C₁₋₃alkyl,
diC₁₋₄alkylaminoC₁₋₄alkyl,
aminoC₁₋₆alkyl,
—CN,
heterocycloalkyl,
heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, oxo, —NO₂, —NH₂ and —CN,
—NO₂,
—NH₂,
—N(H)C₁₋₃alkyl, and
—N(C₁₋₃alkyl)2,
R² is selected from:
aryl,
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN,
heteroaryl,
heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
bicycloheteroaryl,
bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, cycloalkyl, —OC(H)F₂, —C(H)F₂, —OCH₂F, —CH₂F, —CHF₂, —OCF₃, —CN, and cycloalkyl;
R³, R⁴, R⁵, and R⁶ are each independently selected from hydrogen, fluoro, chloro, bromo, iodo, —CF₃, and —CH₃; and
R⁷ is selected from: hydrogen, C₁₋₆alkyl, cycloalkyl, aminoC₁₋₆alkyl —CF₃, —CH₃, fluoro, chloro, bromo and iodo; and
X is O, S, C(═O), NR¹⁰⁰, CR²⁰⁰R³⁰⁰,
where R¹⁰⁰ is selected from hydrogen, C₁₋₆alkyl;
R²⁰⁰ and R³⁰⁰ are independently selected from hydrogen, —CH₃,
—CF₃, —OH, and —NH₂,
or R²⁰⁰ and R³⁰⁰ taken together with the carbon atoms to which they are attached represent a 3 or 4 member cycloalkyl;
or a pharmaceutically acceptable salt thereof.

2. The method of claim 1 wherein the mammal is a human.

3. The method of inhibiting PERK activity in a mammal in need thereof, which comprises administering to such mammal a therapeutically effective amount of a compound of Formula (I), as described in claim 1 or a pharmaceutically acceptable salt thereof.

4. The method of claim 3 wherein the mammal is a human.

5. A compound according to Formula (II): 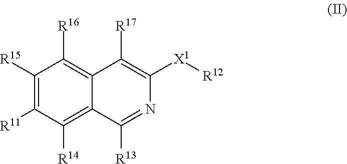
wherein:
R¹¹ is selected from:
bicycloheteroaryl,
substituted bicycloheteroaryl,
heteroaryl, and
substituted heteroaryl,
where said substituted bicycloheteroaryl and said substituted heteroaryl are substituted with from one to five substituents independently selected from:
fluoro,
chloro,
bromo,
iodo,
C₁₋₆alkyl,
C₁₋₆alkyl substituted with from 1 to 5 substituents independently selected from: fluoro, chloro, bromo, iodo, C₁₋₄alkyloxy, —OH, C₁₋₄alkyl, cycloalkyl, —COOH, —CF₃, —NO₂, —NH₂ and —CN,
—OH,
hydroxyC₁₋₆alkyl,
—COOH,
tetrazole,
cycloalkyl,
oxo,
—OC₁₋₆alkyl,
—CF₃,
—CF₂H,
—CFH₂,
—CONH₂,
—CON(H)C₁₋₃alkyl,
diC₁₋₄alkylaminoC₁₋₄alkyl,
aminoC₁₋₆alkyl,
—CN,
heterocycloalkyl,
heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, oxo, —NO₂, —NH₂ and —CN,
—NO₂,
—NH₂,
—N(H)C₁₋₃alkyl, and
N(C₁₋₃alkyl)₂;
R¹² is selected from:
aryl,
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —CHF₂, —OCF₃, and —CN,
heteroaryl,
heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
bicycloheteroaryl,
bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —CHF₂, —OCF₃, —CN, and cycloalkyl;
R¹³, R¹⁴, R¹⁵, and R¹⁶ are each independently selected from hydrogen, fluoro, chloro, bromo, iodo, —CF₃, and —CH₃; and
R¹⁷ is selected from: hydrogen, C₁₋₆alkyl, cycloalkyl, aminoC₁₋₆alkyl, —CF₃, —CH₃, fluoro, chloro, bromo and iodo; and
X is O, S, C(═O), CR²⁵⁰R³⁵⁰,
R²⁵⁰ and R³⁵⁰ are independently selected from hydrogen, —CH₃,
—CF₃, —OH, —NH₂,
or R²⁵⁰ and R³⁵⁰ taken together with the carbon atoms to which they are attached represent a 3 or 4 member cycloalkyl;
or a pharmaceutically acceptable salt thereof.

6. The compound of claim 5 wherein:
R¹² is selected from:
aryl, and
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂,
—NH₂, —OC(H)F₂, —C(H)F₂, —OCH₂F, —CH₂F, —CHF₂, —OCF₃, and —CN;
or a pharmaceutically acceptable salt thereof.

7. The compound of claim 5 wherein:
R¹¹ is selected from: substituted pyrrolo[2,3-d]pyrimidine, substituted pyrazolo[3,4-d]pyrimidine, and substituted pyrrolo[3,2-c]pyridine;
or a pharmaceutically acceptable salt thereof.

8. The compound of any one of claims 5 to 7 wherein: R¹⁴ is fluoro.
or a pharmaceutically acceptable salt thereof.

9. The compound of claim 4 selected from:
5-(3-Benzylisoquinolin-7-yl)-7-methyl-7H-pyrrolo[2,3-d]pyrimidin-4-amine;
5-(3-(3,5-Dimethylbenzyl)isoquinolin-7-yl)-7-methyl-7H-pyrrolo[2,3-d]pyrimidin-4-amine;
5-(3-Benzyl-8-fluoroisoquinolin-7-yl)-7-methyl-7H-pyrrolo[2,3-d]pyrimidin-4-amine;
5-(3-(3,5-Difluorobenzyl)-8-fluoroisoquinolin-7-yl)-7-methyl-7H-pyrrolo[2,3-d]pyrimidin-4-amine;
7-cyclopropyl-5-(3-(2,3-difluorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-ethyl-7H-pyrrolo[2,3-d]pyrimidin-4-amine;
(7-(4-amino-7-cyclopropyl-7H-pyrrolo[2,3-d]pyrimidin-5-yl)-8-fluoroisoquinolin-3-yl)(3,5-difluorophenyl)methanol;
7-cyclopropyl-5-(3-(3,5-difluorobenzyl)-5-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2,2-difluorocyclopropyl)-7H-pyrrolo[2,3-d]pyrimidin-4-amine;
3-(3-(3,5-difluorobenzyl)-8-fluoroisoquinolin-7-yl)-7-fluoro-1-methyl-1H-pyrrolo[3,2-c]pyridin-4-amine;
5-(3-(3,5-difluorobenzyl)-8-fluoroisoquinolin-7-yl)-7-(oxetan-3-yl)-7H-pyrrolo[2,3-d]pyrimidin-4-amine;
7-cyclopropyl-5-(3-(3,5-difluorobenzyl)-8-fluoro-4-methylisoquinolin-7-yl)-7H-pyrrolo[2,3-d]pyrimidin-4-amine;
(7-(4-amino-7-methyl-7H-pyrrolo[2,3-d]pyrimidin-5-yl)isoquinolin-3-yl)(3,5-dimethylphenyl)methanone;
5-(3-(3,4-difluorobenzyl)-8-fluoroisoquinolin-7-yl)-7-methyl-7H-pyrrolo[2,3-d]pyrimidin-4-amine;
5-(3-(2,5-difluorobenzyl)-8-fluoroisoquinolin-7-yl)-7-methyl-7H-pyrrolo[2,3-d]pyrimidin-4-amine;
5-(8-fluoro-3-(3-fluoro-5-(trifluoromethyl)benzyl)isoquinolin-7-yl)-7-methyl-7H-pyrrolo[2,3-d]pyrimidin-4-amine;
5-(8-fluoro-3-(3-(trifluoromethyl)benzyl)isoquinolin-7-yl)-7-methyl-7H-pyrrolo[2,3-d]pyrimidin-4-amine;
7-cyclopropyl-5-(8-fluoro-3-(3-fluorobenzyl) isoquinolin-7-yl)-7H-pyrrolo[2,3-d]pyrimidin-4-amine;
7-cyclopropyl-5-(8-fluoro-3-(4-fluorobenzyl) isoquinolin-7-yl)-7H-pyrrolo[2,3-d]pyrimidin-4-amine;
7-cyclopropyl-5-(3-(2,5-dimethylbenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2,2,2-trifluoroethyl)-7H-pyrrolo[2,3-d]pyrimidin-4-amine;
5-(3-(3,5-difluorobenzyl)-8-fluoroisoquinolin-7-yl)-7-isopropyl-7H-pyrrolo[2,3-d]pyrimidin-4-amine;
5-(3-(3,5-difluorobenzyl)-8-fluoroisoquinolin-7-yl)-2,7-dimethyl-7H-pyrrolo[2,3-d]pyrimidin-4-amine;
7-cyclopropyl-5-(3-(3,5-difluorobenzyl)-8-fluoroisoquinolin-7-yl)-7H-pyrrolo[2,3-d]pyrimidin-4-amine;
3-(3-(3,5-difluorobenzyl)-8-fluoroisoquinolin-7-yl)-1-methyl-1H-pyrrolo[3,2-c]pyridin-4-amine;
7-cyclopropyl-5-(3-(3,5-difluorobenzyl)-8-fluoroisoquinolin-7-yl)-2-methyl-7H-pyrrolo[2,3-d]pyrimidin-4-amine;
1-cyclopropyl-3-(3-(3,5-difluorobenzyl)-8-fluoroisoquinolin-7-yl)-1H-pyrazolo[3,4-d]pyrimidin-4-amine;
7-cyclopropyl-5-(3-(3-(3,5-difluorophenyl)(methoxy)methyl)-8-fluoroisoquinolin-7-yl)-7H-pyrrolo[2,3-d]pyrimidin-4-amine;
7-(2-(2-aminoethoxy)ethyl)-5-(3-(3,5-difluorobenzyl)-8-fluoroisoquinolin-7-yl)-7H-pyrrolo[2,3-d]pyrimidin-4-amine;
7-(2-aminoethyl)-5-(3-(3,5-difluorobenzyl)-8-fluoroisoquinolin-7-yl)-7H-pyrrolo[2,3-d]pyrimidin-4-amine;
7-cyclopropyl-5-(3-(3-ethynyl-5-fluorobenzyl)-8-fluoroisoquinolin-7-yl)-7H-pyrrolo[2,3-d]pyrimidin-4-amine;
7-cyclopropyl-5-(3-(2,5-difluorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(1-methylpiperidin-4-yl)-7H-pyrrolo[2,3-d]pyrimidin-4-amine;
5-(3-(3,5-difluorobenzyl)-8-fluoroisoquinolin-7-yl)-7-(2-morpholinoethyl)-7H-pyrrolo[2,3-d]pyrimidin-4-amine;
5-(3-(5-chloro-2-methylbenzyl)-8-fluoroisoquinolin-7-yl)-7-cyclopropyl-7H-pyrrolo[2,3-d]pyrimidin-4-amine;
7-cyclopropyl-5-(8-fluoro-3-(2-methylbenzyl)isoquinolin-7-yl)-7H-pyrrolo[2,3-d]pyrimidin-4-amine;
5-(3-(3,5-difluorobenzyl)-8-fluoroisoquinolin-7-yl)-7-(1-methylazetidin-3-yl)-7H-pyrrolo[2,3-d]pyrimidin-4-amine;
7-cyclopropyl-5-(3-(1-(3,5-difluorophenyl)ethyl)-8-fluoroisoquinolin-7-yl)-7H-pyrrolo[2,3-d]pyrimidin-4-amine;
7-cyclopropyl-5-(8-fluoro-3-(2-fluoro-5-(trifluoromethyl)benzyl)isoquinolin-7-yl)-7H-pyrrolo[2,3-d]pyrimidin-4-amine;
5-(3-(3,5-difluorobenzyl)isoquinolin-7-yl)-7-methyl-7H-pyrrolo[2,3-d]pyrimidin-4-amine;
5-(3-(3-chlorobenzyl)-8-fluoroisoquinolin-7-yl)-7-cyclopropyl-7H-pyrrolo[2,3-d]pyrimidin-4-amine;
5-(3-(2-chlorobenzyl)-8-fluoroisoquinolin-7-yl)-7-cyclopropyl-7H-pyrrolo[2,3-d]pyrimidin-4-amine;
7-cyclopropyl-5-(8-fluoro-3-(3-fluoro-5-methylbenzyl)isoquinolin-7-yl)-7H-pyrrolo[2,3-d]pyrimidin-4-amine;
7-cyclopropyl-5-(3-(3,5-dichlorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2-(dimethylamino)ethyl)-7H-pyrrolo[2,3-d]pyrimidin-4-amine;
5-(8-fluoro-3-(3-fluorobenzyl)isoquinolin-7-yl)-7-methyl-7H-pyrrolo[2,3-d]pyrimidin-4-amine;
5-(3-(3-chlorobenzyl)-8-fluoroisoquinolin-7-yl)-7-methyl-7H-pyrrolo[2,3-d]pyrimidin-4-amine;
7-cyclobutyl-5-(3-(3,5-difluorobenzyl)-8-fluoroisoquinolin-7-yl)-7H-pyrrolo[2,3-d]pyrimidin-4-amine;
5-(3-(3-chloro-2-fluorobenzyl)-8-fluoroisoquinolin-7-yl)-7-cyclopropyl-7H-pyrrolo[2,3-d]pyrimidin-4-amine;
7-cyclopropyl-5-(3-(2,3-difluorobenzyl)-8-fluoroisoquinolin-7-yl)-7H-pyrrolo[2,3-d]pyrimidin-4-amine;
7-cyclopropyl-5-(8-fluoro-3-((5-fluoropyridin-3-yl)methyl) isoquinolin-7-yl)-7H-pyrrolo[2,3-d]pyrimidin-4-amine;
7-(cyclopropylmethyl)-5-(3-(3,5-difluorobenzyl)-8-fluoroisoquinolin-7-yl)-7H-pyrrolo[2,3-d]pyrimidin-4-amine;
5-(3-(3,5-difluorobenzyl)-8-fluoroisoquinolin-7-yl)-7-(2-methoxyethyl)-7H-pyrrolo[2,3-d]pyrimidin-4-amine;
5-(3-(3,5-difluorobenzyl)-8-fluoroisoquinolin-7-yl)-7-((3-methyloxetan-3-yl)methyl)-7H-pyrrolo[2,3-d]pyrimidin-4-amine;
7-cyclopropyl-5-(3-(3,5-difluorobenzyl)-8-fluoroisoquinolin-7-yl)-6-methyl-7H-pyrrolo[2,3-d]pyrimidin-4-amine;
5-(3-(3,5-difluorobenzyl)-8-fluoroisoquinolin-7-yl)pyrrolo[2,1-f][1,2,4]triazin-4-amine;
5-(3-(3,5-difluorobenzyl)-8-fluoroisoquinolin-7-yl)-7-ethyl-6-methyl-7H-pyrrolo[2,3-d]pyrimidin-4-amine; and
5-(3-(3-chloro-5-fluorobenzyl)-8-fluoroisoquinolin-7-yl)-7-cyclopropyl-7H-pyrrolo[2,3-d]pyrimidin-4-amine;
or a pharmaceutically acceptable salt thereof.

10. A pharmaceutical composition comprising a compound of Formula (II) according to claim 5 or a pharmaceutically acceptable salt thereof and a pharmaceutically acceptable excipient.

11. (canceled)

12. A method of treating a disease selected from: cancer, pre-cancerous syndromes, as Alzheimer's disease, spinal cord injury, traumatic brain injury, ischemic stroke, stroke, Parkinson disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation, in a human in need thereof, which comprises administering to such human a therapeutically effective amount of a compound of Formula (II) according to claim 5 or a pharmaceutically acceptable salt thereof.

13-15. (canceled)

16. The method of inhibiting PERK activity in a human in need thereof, which comprises administering to such human a therapeutically effective amount of a compound of Formula (II) according to claim 5 or a pharmaceutically acceptable salt thereof.

17. (canceled)

18. A method of treating cancer in a mammal in need thereof, which comprises: administering to such mammal a therapeutically effective amount of
a) a compound of Formula (I), as described in claim 1, or a pharmaceutically acceptable salt thereof; and
b) at least one anti-neoplastic agent.

19-21. (canceled)

22. The method according to claim 13 wherein said cancer is selected from: breast cancer, inflammatory breast cancer, ductal carcinoma, lobular carcinoma, colon cancer, pancreatic cancer, insulinomas, adenocarcinoma, ductal adenocarcinoma, adenosquamous carcinoma, acinar cell carcinoma, glucagonoma, skin cancer, melanoma, metastatic melanoma, lung cancer, small cell lung cancer, non-small cell lung cancer, squamous cell carcinoma, adenocarcinoma, large cell carcinoma, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid,
lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia,
malignant lymphoma, hodgkins lymphoma, non-hodgkins lymphoma, lymphoblastic T cell lymphoma, Burkitt's lymphoma, follicular lymphoma,
neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

23. The method according to claim 13 wherein said pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

24. A method of treating or lessening the severity of ocular diseases in a human in need thereof, which comprises administering to such human a therapeutically effective amount of a compound of Formula II, as described in claim 5 or a pharmaceutically acceptable salt thereof.

25. A method according to claim 24 wherein the ocular disease is selected from: rubeosis irides; neovascular glaucoma; pterygium; vascularized glaucoma filtering blebs; conjunctival papilloma; choroidal neovascularization associated with age-related macular degeneration (AMD), myopia, prior uveitis, trauma, or idiopathic; macular edema; retinal neovascularization due to diabetes; age-related macular degeneration (AMD); macular degeneration (AMD); ocular ischemic syndrome from carotid artery disease; ophthalmic or retinal artery occlusion; sickle cell retinopathy; retinopathy of prematurity; Eales Disease; and VonHippel-Lindau syndrome.

26. (canceled)

27. A method of treating or lessening the severity of neurodegeneration in a human in need thereof, which comprises administering to such human a therapeutically effective amount of a compound of Formula (II), as described in claim 5 or a pharmaceutically acceptable salt thereof.

28. A method of preventing organ damage during the transportation of organs for transplantation, which comprises adding a compound of Formula (II) as described in claim 5 or a pharmaceutically acceptable salt thereof to the solution housing the organ during transportation.

29. (canceled)

30. A combination comprising:
a) a compound of Formula (I), as described in claim 1, or a pharmaceutically acceptable salt thereof; and
b) an ATF-4 modulating compound.
